(19) United States
(12) Patent Application Publication
(10) Pub. No.: US 2022/0267409 A1
(43) Pub. Date: Aug. 25, 2022

(54) HETERODIMERS AND METHODS OF USE THEREOF

(71) Applicants: KAHR Medical Ltd., Jerusalem (IL); Thomas Jefferson University, Philadelphia, PA (US)

(72) Inventors: Mark L. TYKOCINSKI, Merion Station, PA (US); Ami TAMIR, Rehovot (IL); Edwin BREMER, Groningen (NL)

(73) Assignees: KAHR Medical Ltd., Jerusalem (IL); Thomas Jefferson University, Philadelphia, PA (US)

(21) Appl. No.: 17/626,185

(22) PCT Filed: Jul. 8, 2020
(86) PCT No.: PCT/IL2020/050762

§ 371 Date: Jan. 11, 2022

Related U.S. Application Data
(60) Provisional application No. 62/872,741, filed on Jul. 11, 2019.

(51) Int. Cl. C07K 14/705; A61K 35/17; A61P 35/00

(52) U.S. Cl. CPC C07K 14/70521; C07K 14/70596; C07K 14/70578; C07K 14/70575; A61K 38/00; A61K 35/17; A61P 35/00; C07K 2319/30; C07K 14/7056

(57) ABSTRACT

Heterodimers are provided. Accordingly, there is provided a heterodimer comprising a dimerizing moiety attached to at least one amino acid sequence of at least one type I membrane protein capable of at least binding a natural ligand or receptor of said at least one type I membrane protein and to at least one amino acid sequence of at least one type II membrane protein capable of at least binding a natural ligand or receptor of said at least one type II membrane protein. Also provided are nucleic acid constructs and systems encoding the heterodimer, host-cells expressing same and methods of use thereof.

### RELATED APPLICATION

This application claims priority from U.S. Patent Application No. 62/872,741 filed on Jul. 11, 2019, the contents of which are incorporated herein by reference in their entirety.

### SEQUENCE LISTING STATEMENT

The ASCII file, entitled 82913 SequenceListing.txt, created on Jul. 7, 2020, comprising 347,293 bytes, submitted concurrently with the filing of this application is incorporated herein by reference.

### BACKGROUND OF THE INVENTION

The present invention, in some embodiments thereof, relates to heterodimers and methods of use thereof.

Dual Signaling Proteins (DSP), also known as Signal-Converting-Proteins (SCP), are bi-functional fusion proteins that link an extracellular portion of a type I membrane protein (extracellular amino-terminus), to an extracellular portion of a type II membrane protein (extracellular carboxyl-terminus), forming a fusion protein (polypeptide chain) with two active sides (see e.g. U.S. Pat. Nos. 7,569,663 and 8,039,437). Several such DSPs have been disclosed in the art, including for example PD1-4-1BBL and SIRPa-4-1BBL (see e.g. International Patent Application Publication No. WO2018/127919 and WO2018/127917). By binding to their native corresponding ligands or receptors, DSPs can have targeting and/or functional properties affecting e.g. a signaling cascade, growth, survival and activity of cells, depending on their composition. Thus, for example, a DSP can be designed such that one arm serves for selective targeting to a tumor site or tumor microenvironment while the other arm serves as an immune modulator. This unique composition of DSPs, like the PD1-4-1BBL and SIRPa-4-1BBL, can facilitate targeted activation of adaptive immunity at a tumor site. The platform technology is adaptable to most checkpoint targets, potentially improving efficacy while maintaining a favorable risk/benefit ratio.

### SUMMARY OF THE INVENTION

According to an aspect of some embodiments of the present invention there is provided a heterodimer comprising a dimerizing moiety attached to at least one amino acid sequence of at least one type I membrane protein capable of at least binding a natural ligand or receptor of the at least one type I membrane protein and to at least one amino acid sequence of at least one type II membrane protein capable of at least binding a natural ligand or receptor of the at least one type II membrane protein.

According to some embodiments of the invention, the dimerizing moiety is a proteinaceous moiety.

According to some embodiments of the invention, monomers of the heterodimer are not covalently attached.

According to some embodiments of the invention, the dimerizing moiety is an Fc domain of an antibody or a fragment thereof.

According to some embodiments of the invention, at least one type I membrane protein is selected from the group consisting of PD1, SIRPa, LAG3, BTN3A1, CD27, CD80, CD86, ENG, NLGN4X, CD84, TIGIT, CD40, IL-8, IL-10, CD164, LY6G6F, CD28, CTLA4, BTLA, LILRB1, LILRB2, TYROBP, ICOS, VEGFA, CSF1, CSF1R, VEGFB, BMP2, BMP3, GDNF, PDGFC, PDGFD, RAETIE, CD155, CD166, MICA, NRG1, HVEM, DR3, TEK, TGFB1, LY96, CD96, KIT, CD244, GFER and SIGLEC.

According to some embodiments of the invention, the at least one type I membrane protein is selected from the group consisting of PD1, SIRPa, LAG3, BTN3A1, CD27, CD80, CD86, ENG, NLGN4X, CD84, TIGIT, CD40, IL-8, IL-10, CD164, LY6G6F, CD28, CTLA4, BTLA, LILRB1, LILRB2, TYROBP, ICOS, VEGFA, CSF1, CSF1R, VEGFB, BMP2, BMP3, GDNF, PDGFC, PDGFD, RAETIE, CD155, CD166, MICA, NRG1, HVEM, DR3, TEK, TGFB1, LY96, CD96, KIT, CD244, and GFER

According to some embodiments of the invention, the at least one type I membrane protein is selected from the group consisting of PD1, SIRPa, LAG3, TIGIT, LILRB1/2, CSF1, CSF1R and TGFB1.

According to some embodiments of the invention, the at least one type I membrane protein is selected from the group consisting of PD1, SIRPa, TIGIT, LILRB2 and SIGLEC.

According to some embodiments of the invention, the at least one type I membrane protein is selected from the group consisting of PD1 and SIRPa.

According to some embodiments of the invention, the at least one type II membrane protein is selected from the group consisting of 4-1BBL, FasL, TRAIL, TNF-alpha, TNF-beta, OX40L, CD40L, CD27L, CD30L, RANKL, TWEAK, APRIL, BAFF, LIGHT, VEGI, GITRL, EDA1/2, Lymphotoxin alpha and Lymphotoxin beta.

According to some embodiments of the invention, the at least one type II membrane protein is selected from the group consisting of 4-1BBL, OX40L, CD40L, LIGHT and GITRL.

According to some embodiments of the invention, the at least one type II membrane protein is selected from the group consisting of 4-1BBL and CD40L.

According to some embodiments of the invention, the at least one type II membrane protein is 4-1BBL.

According to some embodiments of the invention, at least one of the type I membrane protein and the type II membrane protein is an immune modulator.

According to some embodiments of the invention, the heterodimer comprises a first monomer comprising the at least one amino acid sequence of the at least one type I membrane protein and the at least one amino acid sequence of the at least one type II membrane protein.

According to some embodiments of the invention, the heterodimer comprises a first monomer comprising the at least one amino acid sequence of the at least one type II membrane protein and a second monomer comprising the at least one amino acid sequence of the at least one type I membrane protein.

According to some embodiments of the invention, the at least one amino acid sequence of the at least one type I membrane protein comprises at least two amino acid sequences of the at least one type I membrane protein; and the heterodimer comprises a first monomer comprising at least one of the at least two amino acid sequences of the at least one type I membrane protein and the at least one amino acid sequence of the at least one type II membrane protein and a second monomer comprising at least one of the at least two amino acid sequences of the at least one type I membrane protein.

According to some embodiments of the invention, the at least one of the at least two amino acid sequences of the at least one type I membrane protein of the first monomer and the at least one of the at least two amino acid sequence of the type I membrane protein of the second monomer are identical.

According to some embodiments of the invention, the at least one of the at least two amino acid sequences of the at least one type I membrane protein of the first monomer and the at least one of the at least two amino acid sequences of the at least one type I membrane protein of the second monomer are distinct.

According to some embodiments of the invention, the at least one amino acid sequence of the at least one type I membrane protein comprises at least two amino acid sequences of at least two type I membrane proteins; and the heterodimer comprises a first monomer comprising at least one of the at least two amino acid sequences of the at least two type I membrane proteins and the at least one amino acid sequence of the at least one type TT membrane protein and a second monomer comprising at least one of the at least two amino acid sequences of the at least two type I membrane proteins.

According to some embodiments of the invention, the at least one amino acid sequence of the type I membrane protein comprises at least two amino acid sequences of the type I membrane protein, the type I membrane protein is PD1, the type II membrane protein is 4-1BBL, and the heterodimer comprises a first monomer comprising at least one of the at least two amino acid sequences of the PD1 and the at least one amino acid sequence of the 4-1BBL and a second monomer comprising at least one of the at least two amino acid sequences of the PD1.

According to some embodiments of the invention, the at least one amino acid sequence of the type I membrane protein comprises at least two amino acid sequences of the type I membrane protein, the type I membrane protein is LILRB2, the type II membrane protein is 4-1BBL, and the heterodimer comprises a first monomer comprising at least one of the at least two amino acid sequences of the LILRB2 and the at least one amino acid sequence of the 4-1BBL and a second monomer comprising at least one of the at least two amino acid sequences of the LILRB2.

According to some embodiments of the invention, the at least one amino acid sequence of the type I membrane protein comprises at least two amino acid sequences of the type I membrane protein, the type I membrane protein is LILRB2, the type II membrane protein is CD40L, and the heterodimer comprises a first monomer comprising at least one of the at least two amino acid sequences of the LILRB2 and the at least one amino acid sequence of the CD40L and a second monomer comprising at least one of the at least two amino acid sequences of the LILRB2.

According to some embodiments of the invention, the at least one amino acid sequence of the type I membrane protein comprises at least two amino acid sequences of at least two type I membrane proteins, the at least two type I membrane proteins comprise PD1 and SIRPa, the type II membrane protein is 4-1BBL, and the heterodimer comprises a first monomer comprising the amino acid sequence of the SIRPa and the amino acid sequence of the 4-1BBL and a second monomer comprising the amino acid sequence of the PD1.

According to some embodiments of the invention, the at least one amino acid sequence of the type I membrane protein comprises at least two amino acid sequences of at least two type I membrane proteins, the at least two type I membrane proteins comprise PD1 and SIRPa, the type II membrane protein is CD40L, and the heterodimer comprises a first monomer comprising the amino acid sequence of the SIRPa and the amino acid sequence of the CD40L and a second monomer comprising the amino acid sequence of the PD1.

According to some embodiments of the invention, the at least one amino acid sequence of the type I membrane protein comprises at least two amino acid sequences of at least two type I membrane proteins, the at least two type I membrane proteins comprise LILRB2 and SIRPa, the type II membrane protein is 4-1BBL, and the heterodimer comprises a first monomer comprising the amino acid sequence of the SIRPa and the amino acid sequence of the 4-1BBL and a second monomer comprising the amino acid sequence of the LILRB2.

According to some embodiments of the invention, the at least one amino acid sequence of the type I membrane protein comprises at least two amino acid sequences of at least two type I membrane proteins, the at least two type I membrane proteins comprise LILRB2 and SIRPa, the type II membrane protein is CD40L, and the heterodimer comprises a first monomer comprising the amino acid sequence of the SIRPa and the amino acid sequence of the CD40L and a second monomer comprising the amino acid sequence of the LILRB2.

According to some embodiments of the invention, the at least one amino acid sequence of the type I membrane protein comprises at least two amino acid sequences of at least two type I membrane proteins, the at least two type I membrane proteins comprise LILRB2 and PD1, the type II membrane protein is 4-1BBL, and the heterodimer comprises a first monomer comprising the amino acid sequence of the PD1 and the amino acid sequence of the 4-1BBL and a second monomer comprising the amino acid sequence of the LILRB2.

According to some embodiments of the invention, the at least one amino acid sequence of the type I membrane protein comprises at least two amino acid sequences of at least two type I membrane proteins, the at least two type I membrane proteins comprise LILRB2 and PD1, the type II membrane protein is CD40L, and the heterodimer comprises a first monomer comprising the amino acid sequence of the PD1 and the amino acid sequence of the CD40L and a second monomer comprising the amino acid sequence of the LILRB2.

According to some embodiments of the invention, at least one amino acid sequence of the type I membrane protein comprises at least two amino acid sequences of at least two type I membrane proteins, the at least two type I membrane proteins comprise SIGLEC and PD1, the type II membrane protein is 4-1BBL, and the heterodimer comprises a first monomer comprising the amino acid sequence of the PD1 and the amino acid sequence of the 4-1BBL and a second monomer comprising the amino acid sequence of the SIGLEC.

According to some embodiments of the invention, the at least one amino acid sequence of the type I membrane protein comprises at least two amino acid sequences of at least two type I membrane proteins, the at least two type I membrane proteins comprise SIGLEC and PD1, the type II membrane protein is CD40L, and the heterodimer comprises a first monomer comprising the amino acid sequence of the PD1 and the amino acid sequence of the CD40L and a second monomer comprising the amino acid sequence of the SIGLEC.

According to some embodiments of the invention, the at least one amino acid sequence of the type I membrane protein comprises at least two amino acid sequences of at least two type I membrane proteins, the at least two type I membrane proteins comprise TIGIT and PD1, the type II membrane protein is 4-1BBL, and the heterodimer comprises a first monomer comprising the amino acid sequence of the PD1 and the amino acid sequence of the 4-1BBL and a second monomer comprising the amino acid sequence of the TIGIT.

According to some embodiments of the invention, the at least one amino acid sequence of the type I membrane protein comprises at least two amino acid sequences of at least two type I membrane proteins, the at least two type I membrane proteins comprise TIGIT and PD1, the type II membrane protein is CD40L, and the heterodimer comprises a first monomer comprising the amino acid sequence of the PD1 and the amino acid sequence of the CD40L and a second monomer comprising the amino acid sequence of the TIGIT.

According to some embodiments of the invention, the at least one amino acid sequence of the type I membrane protein comprises at least two amino acid sequences of at least two type I membrane proteins, the at least two type I membrane proteins comprise TIGIT and PD1, the type II membrane protein is 4-1BBL, and the heterodimer comprises a first monomer comprising the amino acid sequence of the TIGIT and the amino acid sequence of the 4-1BBL and a second monomer comprising the amino acid sequence of the PD1.

According to some embodiments of the invention, the at least one amino acid sequence of the type I membrane protein comprises at least two amino acid sequences of at least two type I membrane proteins, the at least two type I membrane proteins comprise TIGIT and PD1, the type II membrane protein is CD40L, and the heterodimer comprises a first monomer comprising the amino acid sequence of the TIGIT and the amino acid sequence of the CD40L and a second monomer comprising the amino acid sequence of the PD1.

According to some embodiments of the invention, the SIGLEC is SIGLEC10.

According to some embodiments of the invention, the at least one amino acid sequence of the at least one type I membrane protein is attached to an N-terminus of the proteinaceous dimerizing moiety and the at least one amino acid sequence of the at least one type II membrane protein is attached to a C-terminus of the proteinaceous dimerizing moiety.

According to an aspect of some embodiments of the present invention there is provided a nucleic acid construct or system comprising at least one polynucleotide encoding the heterodimer, and a regulatory element for directing expression of the polynucleotide in a host cell.

According to an aspect of some embodiments of the present invention there is provided a host cell comprising the heterodimer or the nucleic acid construct or system.

According to an aspect of some embodiments of the present invention there is provided a method of producing a heterodimer, the method comprising expressing in a host cell a nucleic acid construct or system encoding the heterodimer.

According to some embodiments of the invention, the method comprising adding the dimerizing moiety to the at least one amino acid sequence of the at least one type I membrane protein and the at least one amino acid sequence of the at least one type II membrane protein.

According to some embodiments of the invention, the method comprising isolating the heterodimer.

According to an aspect of some embodiments of the present invention there is provided a method of treating a disease that can benefit from treatment with the heterodimer, the method comprising administering to a subject in need thereof the heterodimer, a nucleic acid construct or system encoding same or a host cell comprising same, thereby treating the disease in the subject.

According to an aspect of some embodiments of the present invention there is provided a method of treating a disease that can benefit from modulating immune cells, the method comprising administering to a subject in need thereof the heterodimer, a nucleic acid construct or system encoding same or a host cell comprising same, thereby treating the disease in the subject.

According to some embodiments of the invention, the method further comprising administering to the subject a therapeutic agent for treating the disease.

According to an aspect of some embodiments of the present invention there is provided the heterodimer, a nucleic acid construct or system encoding same or a cell comprising same for use in treating a disease that can benefit from treatment with the heterodimer.

According to an aspect of some embodiments of the present invention there is provided the heterodimer, a nucleic acid construct or system encoding same or a host cell comprising same for use in treating a disease that can benefit from modulating immune cells.

According to some embodiments of the invention, the composition further comprising a therapeutic agent for treating the disease.

According to some embodiments of the invention, the therapeutic agent for treating the disease comprises an antibody.

According to some embodiments of the invention, cells of the disease express a ligand or a receptor of the type I membrane protein.

According to some embodiments of the invention, cells of the disease express a ligand or a receptor of the type II membrane protein.

According to some embodiments of the invention, the disease is cancer.

According to some embodiments of the invention, the cancer is selected from the group consisting of lymphoma, leukemia and carcinoma.

According to an aspect of some embodiments of the present invention there is provided a method of modulating activity of immune cells, the method comprising in-vitro activating immune cells in the presence of the heterodimer, a nucleic acid construct or system encoding same or a host cell comprising same.

According to some embodiments of the invention, the activating is in the presence of cells expressing a ligand or a receptor of the type I membrane protein or the type II membrane protein or exogenous ligand or a receptor of the type I membrane protein or the type II membrane protein.

According to some embodiments of the invention, the modulating is activating.

According to some embodiments of the invention, the modulating is inhibiting.

According to some embodiments of the invention, the immune cells comprise T cells.

Unless otherwise defined, all technical and/or scientific terms used herein have the same meaning as commonly understood by one of ordinary skill in the art to which the invention pertains.

Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of non-limiting examples of possible arrangements/conformations of a heterodimer.

FIG. 2 shows schematic representations of the PD1-sc3x4-1BBL (3 repeats of extracellular domain of 4-1BBL) heterodimers referred to herein as “DSP305” (SEQ ID NOs: 79 and 81) and “DSP305_V1” (SEQ ID NOs: 79 and 83).

FIGS. 3A-H demonstrate the predicted 3D structure of the PD1-sc3x4-1BBL heterodimers DSP305 (SEQ ID NOs: 79 and 81) and DSP305_V1 (SEQ ID NOs: 79 and 83). FIG. 3A is a schematic 3D model and FIG. 3B is a full atomic 3D model of DSP305 (SEQ ID NOs: 79 and 81). PD1 sequences (for both ‘knob’ and ‘hole’) are represented in a dark grey ribbons display (right-hand side). hIgG4 of the ‘knob’ sequence is represented in white ribbons in the middle of the FIG. 3A. hIgG4 of the ‘hole’ sequence is represented in grey ribbons in the middle of the FIG. 3A. 4-1BB-L is represented in dark grey ribbons (left-hand side). ‘Spacer’/‘linker’ segments are represented in grey and white ribbons between the structural elements of PD1, hIgG4 and 4-1BBL. The hinge cysteine residues of the hIgG4 Fc domain (which stabilize the complex) are represented in a CPK representation. FIG. 3C is a schematic 3D model and FIG. 3D is a full atomic 3D models of DSP305 (SEQ ID NOs: 79 and 81) in the presence of its ligands (PDL1 and 4-1BB). The different domains are represented by different ribbons marked as in FIG. 3A. In addition, PDL1 bound to PD1 is represented in grey ribbons (right-hand side) and three 4-1BB domains are represented in grey ribbons in complex with 4-1BBL (left-hand side). FIG. 3E is a schematic 3D model and FIG. 3F is a full atomic 3D model of DSP305_V1 (SEQ ID NOs: 79 and 83). The different domains are represented by different ribbons marked as in FIG. 3A. FIG. 3G is a schematic 3D model and FIG. 3H is a full atomic 3D model of DSP305_V1 (SEQ ID NOs: 79 and 83) in the presence of its ligands (PDL1 and 4-1BB). The different domains are representing by different ribbons marked as in FIG. 3A. In addition, PDL1 bound to PD1 is represented in grey ribbons (right-hand side) and three 4-1BB domains are represented in grey ribbons in complex with 4-1BBL (left-hand side).

FIG. 4 is a photograph of SDS poly acrylamide gel electrophoresis (SDS-PAGE) analysis of DSP305 and DSP305_V1 separated under reducing and/or non-reducing conditions. The samples presented in the figures are of crude (non-purified) or protein-A purified-five days-supernatant. The supernatants are from Expi293F cells that were transfected with plasmids encoding the heterodimers as indicated. The control sup is of a five days-supernatant of non-transfected Expi293F cells.

FIG. 5 shows schematic representations of the PD1-SIRPa-sc3x4-1BBL heterodimers referred to herein as “TSP111” (SEQ ID NOs: 85 and 81), “TSP111_V1” (SEQ ID NOs: 89 and 91) and “TSP111_V2” (SEQ ID NOs: 85 and 83).

FIGS. 6A-D demonstrate the predicted 3D structure of PD1-SIRPa-sc3x4-1BBL heterodimer TSP111 (SEQ ID NOs: 85 and 81). FIG. 6A is a schematic 3D model and FIG. 6B is a full atomic 3D model of TSP111 (SEQ ID NOs: 85 and 81). PD1 (in the ‘knob’ chain) is represented in a dark grey ribbons display (lower right-hand side). SIRPa (in the ‘hole’ chain) is represented in a dark grey ribbons display (upper right-hand side). hIgG4 of the ‘knob’ sequence is represented in white ribbons in the middle of the figure. hIgG4 of the ‘hole’ sequence is represented in grey ribbons in the middle of the figure. 4-1BBL is represented in dark grey ribbons (left-hand side). ‘Spacer’/‘linker’ segments are represented in grey and white ribbons between the structural elements of PD1, hIgG4 and 4-1BBL. The hinge cysteine residues of the hIgG4 Fe domain (which stabilize the complex) are represented in a CPK representation. FIG. 6C is a schematic 3D model and FIG. 6D is a full atomic 3D model of TSP111 (SEQ ID NOs: 85 and 81) in the presence of its ligands (CD47, PDL1 and 4-1BB). The different domains are represented by different ribbons marked as in FIG. 6A. In addition, PDL1 bound to PD1 is represented in grey ribbons (lower right-hand side), CD47 (SIRPa receptor) is represented in grey ribbons (upper right-hand side) and three 4-1BB receptors are represented in grey ribbons in complex with 4-1BBL (left-hand side).

FIG. 7 is a photograph of SDS-PAGE analysis of TSP111, TSP111_V1 and TSP111_V2, separated under reducing and/or non-reducing conditions. The samples presented in the figures are of crude (non-purified) or protein-A purified-five days-supernatant. The supernatants are from Expi293F cells that were transfected with plasmids encoding to the heterodimers as indicating. The control sup is of a five days-supernatant of non-transfected Expi293F cells.

FIGS. 8A-D demonstrate binding of the PD1-sc3x4-1BBL heterodimer DSP305 (SEQ ID NOs: 79 and 81) to its ligands expressed on the surface of cells. FIG. 8A presents flow cytometric analysis-histogram demonstrating expression of the PDL1 receptor on DLD1-PDL1 cell line. The surface expression level of PDL1 was determined by immuno-staining of DLD1.20 WT and PDL1 overexpressing cell lines (DLD1-PDL1) with an anti-PDL1 antibody, followed by flow cytometric analysis. GMFI values are presented as determined by FACS detecting and FlowJo software analysis. FIG. 8B presents flow cytometric analysis-histogram demonstrating expression of the 4-1BB receptor on HT1080-4-1BB cell line. The surface expression level of 4-1BB was determined by immuno-staining of HT1080 WT and 4-1BB overexpressing cell lines with an anti4-1BB antibody, followed by flow cytometric analysis. GMFI values are presented.

FIG. 8C presents flow cytometric analysis demonstrating binding of DSP305 (SEQ ID NOs: 79 and 81) to DLD1 WT and PDL1 overexpressing cell lines. The binding of the heterodimer to the ligand expressing cell lines was determined by immuno-staining of its 4-1BBL domain using an anti-4-1BBL antibody following incubation of the cells with the indicated heterodimer, followed by flow cytometric analysis. GMFI values are presented and were used to create a binding curve graph with a GraphPad Prism software. FIG. 8D presents flow cytometric analysis demonstrating binding of DSP305 to HT1080 WT and 4-1BB overexpressing cell lines. The binding of the heterodimer to the cell lines was determined by immuno-staining of its PD1 domain using an anti-PD1 antibody following incubation of the cells with the indicated heterodimer, followed by flow cytometric analysis. GMFI values are presented and were used to create a binding curve graph with a GraphPad Prism software.

FIGS. 9A-D demonstrate binding of the PD1-SIRPa-sc3x4-1BBL heterodimer TSP111, to its ligands expressed on the surface of cells. FIG. 9A presents flow cytometric analysis demonstrating binding of TSP111 to DLD1 WT and PDL1 overexpressing cell lines. The binding of the heterodimer to the ligand expressing cell lines was determined by immuno-staining of its 4-1BBL domain using an anti-4-1BBL antibody following incubation of the cells with the indicated heterodimer, followed by flow cytometric analysis. GMFI values are presented and were used to create a binding curve graph with a GraphPad Prism software. FIG. 9B presents flow cytometric analysis demonstrating binding of TSP111 to HT1080 WT and 4-1BB overexpressing cell lines. The binding of the heterodimer protein to the cell lines was determined by immuno-staining of its PD1 domain using an anti-PD1 antibody following incubation of the cells with the indicated heterodimer, followed by flow cytometric analysis. GMFI values are presented and were used to create a binding curve graph with a GraphPad Prism software. FIG. 9C presents flow cytometric analysis-histogram demonstrating expression of the CD47 receptor on CHO-K1-CD47 overexpressing cell line with no expression on the CHO-K1 WT parental cell line. The surface expression level of CD47 was determined by immuno-staining of CHO-K1 WT and CHO-K1-CD47 cell lines with an anti-human-CD47 antibody, followed by flow cytometric analysis. GMFI values are presented as determined by FACS detecting and FlowJo software analysis. FIG. 9D presents flow cytometric analysis demonstrating binding of TSP111 to CHO-K1 WT and CD47 overexpressing cell lines in the absence or presence of an anti-CD47 blocking Ab. The binding of the heterodimer to the ligand expressing cell lines was determined by immuno-staining of its PD1 domain using an anti-PD1 antibody following incubation of the cells with the indicated heterodimer, followed by flow cytometric analysis. GMFI values are presented and were used to create a binding curve graph with a GraphPad Prism software.

FIGS. 10A-B demonstrate simultaneous binding of the PD1-sc3x4-1BBL heterodimer DSP305 and the PD1-SIRPa-sc3x4-1BBL heterodimer TSP111 to, at least, two ligands. Supernatants containing the heterodimers or control supernatant (from non-transfected Expi293F cells) were incubated in PDL1 or CD47 pre-coated 96 wells plate or a mix of both proteins at equal-molar quantity. Following incubation, detection was effected with biotinylated 4-1BBL. FIG. 10A shows binding of DSP305 in a concentration dependent manner to PDL1-coated plates and FIG. 10B demonstrates binding of TSP111 independently to CD47, PDL1 and mixed protein-coated plates. Detection was effected with a TMB substrate according to standard ELISA protocol using a Plate reader (Thermo Scientific, Multiscan FC) at 450 nm, with reference at 540 nm.

FIGS. 11A-C demonstrate activation of 41BB mediated signal transduction by the heterodimers DSP305, TSP111, TSP111_V1 and TSP111_V2. FIGS. 11A-C present IL-8 secretion from HT1080 4-1BB cells incubated in PD1 coated plates in the presence of supernatants containing DSP305 or control supernatant (from non-transfected cells) (FIG. 11A); HT1080 4-1BB cells incubated in plates coated with PD1 or CD47 or a mixture of both proteins in the presence of supernatants containing TSP111, or control supernatant (from non-transfected cells) (FIG. 11B); or HT1080 4-1BB cells incubated in plates coated with CD47 in the presence of supernatants containing TSP111, TSP111_V1, TSP111_V2 or control supernatant (from non-transfected cells) (FIG. 11C). Supernatants were analyzed for IL-8 concentration using IL-8 ELISA kit and a Plate reader (Thermo Scientific, Multiscan FC) at 450 nm, with reference at 540 nm.

FIG. 12A is a schematic representation of the PD1-SIRPα-sc3xCD40L heterodimer referred to herein as “TSP112” (SEQ ID NOs: 81 and 146).

FIGS. 12 B-C demonstrate the predicted 3D structure of PD1-SIRPα-sc3xCD40L heterodimer TSP112 (SEQ ID NOs: 81 and 146). FIG. 12B is a schematic 3D model and FIG. 12C is a full atomic 3D model of TSP112 (SEQ ID NOs: 81 and 146). PD1 (in the ‘knob’ chain) is represented in a dark grey ribbons display (lower right-hand side). SIRPa (in the ‘hole’ chain) is represented in a dark grey ribbons display (upper right-hand side). hIgG4 of the ‘knob’ sequence is represented in white ribbons in the middle of the figure. hIgG4 of the ‘hole’ sequence is represented in grey ribbons in the middle of the figure. CD40L is represented in dark grey ribbons (left-hand side). ‘Spacer’/‘linker’ segments are represented in grey and white ribbons between the structural elements of PD1, SIRPa and hIgG4 and CD40L. The hinge cysteine residues of the hIgG4 Fc domain (which stabilize the complex) are represented in a CPK representation.

FIG. 13A is a schematic representation of the LILRB2-SIRPα-sc3x4-1BBL heterodimer referred to herein as “TSP215” (SEQ ID NOs: 138 and 85).

FIGS. 13B-C demonstrate the predicted 3D structure of LILIRB2-SIRPα-sc3x4-1BBL heterodimer TSP215 (SEQ ID NOs: 138 and 85). FIG. 13B is a schematic 3D model and FIG. 13C is a full atomic 3D model of TSP115 (SEQ ID NOs: 138 and 85). LILRB2 (in the ‘knob’ chain) is represented in a dark grey ribbons display (lower right-hand side). SIRPa (in the ‘hole’ chain) is represented in a dark grey ribbons display (upper right-hand side). hIgG4 of the ‘knob’ sequence is represented in white ribbons in the middle of the figure. hIgG4 of the ‘hole’ sequence is represented in grey ribbons in the middle of the figure. 4-1BBL is represented in dark grey ribbons (left-hand side). ‘Spacer’/‘linker’ segments are represented in grey and white ribbons between the structural elements of LILRB2, SIRPa and hIgG4 and 4-1BBL. The hinge cysteine residues of the hIgG4 Fe domain (which stabilize the complex) are represented in a CPK representation.

FIG. 14A is a schematic representation of the LILRB2-SIRPα-sc3xCD40L heterodimer referred to herein as “TSP217” (SEQ ID NOs: 138 and 146).

FIGS. 14 B-C demonstrate the predicted 3D structure of PD1-SIRPα-sc3xCD40L heterodimer TSP217 (SEQ ID NOs: 138 and 146). FIG. 14B is a schematic 3D model and FIG. 14C is a full atomic 3D model of TSP217 (SEQ ID NOs: 138 and 146). LILRB2 (in the ‘knob’ chain) is represented in a dark grey ribbons display (lower right-hand side). SIRPa (in the ‘hole’ chain) is represented in a dark grey ribbons display (upper right-hand side). hIgG4 of the ‘knob’ sequence is represented in white ribbons in the middle of the figure. hIgG4 of the ‘hole’ sequence is represented in grey ribbons in the middle of the figure. CD40L is represented in dark grey ribbons (left-hand side). ‘Spacer’/‘linker’ segments are represented in grey and white ribbons between the structural elements of LILRB2, SIRPa and hIgG4 and CD40L. The hinge cysteine residues of the hIgG4 Fc domain (which stabilize the complex) are represented in a CPK representation.

FIG. 15A is a schematic representation of the SIGLEC10-PD1-sc3x4-1BBL heterodimer referred to herein as “TSP401” (SEQ ID NOs: 150 and 79).

FIGS. 15B-C demonstrate the predicted 3D structure of SIGLEC10-PD1-sc3x4-1BBL heterodimer “TSP401” (SEQ ID NOs: 150 and 79). FIG. 15B is a schematic 3D model and FIG. 15C is a full atomic 3D model of TSP401 (SEQ ID NOs: 150 and 79). SIGLEC10 sequences (in the ‘knob’ chain) is represented in a dark grey ribbons display (lower right-hand side). PD1 (in the ‘hole’ chain) is represented in dark grey ribbons display (upper right-hand side). hIgG4 of the ‘knob’ sequence is represented in white ribbons in the middle of the figure. hIgG4 of the ‘hole’ sequence is represented in grey ribbons in the middle of the figure. 4-1BB-L is represented in dark grey ribbons (left-hand side). ‘Spacer’/‘linker’ segments are represented in grey and white ribbons between the structural elements of SIGLEC10, PD1 and hIgG4 and 4-1BBL. The hinge cysteine residues of the hIgG4 Fc domain (which stabilize the complex) are represented in a CPK representation.

FIG. 16A is a schematic representation of the TIGIT-PD1-sc3x4-1BBL heterodimer referred to herein as “TSP501” (SEQ ID NOs: 152 and 79).

FIGS. 16B-C demonstrate the predicted 3D structure of TIGIT-PD1-sc3x4-1BBL heterodimer “TSP501” (SEQ ID NOs: 152 and 79). FIG. 16B is a schematic 3D model and FIG. 16C is a full atomic 3D model of TSP501 (SEQ ID NOs: 152 and 79). TIGIT sequences (in the ‘knob’ chain) is represented in a dark grey ribbons display (lower right-hand side). PD1 (in the ‘hole’ chain) is represented in dark grey ribbons display (upper right-hand side). hIgG4 of the ‘knob’ sequence is represented in white ribbons in the middle of the figure. hIgG4 of the ‘hole’ sequence is represented in grey ribbons in the middle of the figure. 4-1BB-L is represented in dark grey ribbons (left-hand side). ‘Spacer’/‘linker’ segments are represented in grey and white ribbons between the structural elements of TIGIT, PD1 and hIgG4 and 4-1BBL. The hinge cysteine residues of the hIgG4 Fc domain (which stabilize the complex) are represented in a CPK representation.

FIG. 17 is a photograph of SDS-PAGE analysis of TSP215, TSP215_V1, TSP214 and TSP214_V1, separated under reducing or non-reducing conditions. The samples presented in the figure are of crude (non-purified)-five days-supernatant. The supernatants are from Expi293F cells that were transfected with plasmids encoding to the heterodimers indicated. The control sup is of a five days-supernatant of non-transfected Expi293F cells.

FIGS. 18A-B show photographs of SDS-PAGE analysis of TSP112, TSP217, DSP218, TSP221, TSP222, TSP401, TSP403, TSP501 and TSP503, separated under reducing (R) or non-reducing (NR) conditions. The samples presented in the figures are of crude (non-purified, FIG. 18A) or protein-A purified (FIG. 18B)-five days-supernatant. The supernatants are from Expi293F cells that were transfected with plasmids encoding to the heterodimers indicated. The control sup is of a five days-supernatant of non-transfected Expi293F cells.

FIGS. 19A-C are photographs of Western Blot analysis of TSP111. The samples presented in the figures are of crude (non-purified)-five days-supernatant. The supernatants are from Expi293F cells that were transfected with plasmids encoding to the heterodimers as indicating. The control sup is of a five days-supernatant of non-transfected Expi293F cells. The supernatants were separated on SDS-PAGE at non-reducing (NR) or reducing (R) conditions, followed by immunoblotting with anti-PD1 (FIG. 19A), anti-SIRPα (FIG. 19B) or anti-4-1BBL (FIG. 19C) antibodies.

FIGS. 20A-C are photographs of a Western Blot analysis of TSP112, TSP401, TSP501 and TSP221. The samples presented in the figures are of crude (non-purified)-five days-supernatant. The supernatants are from Expi293F cells that were transfected with plasmids encoding to the heterodimers indicated. Supernatant samples containing approximately 50 ng of the heterodimers proteins were separated on SDS-PAGE at non-reducing (NR) or reducing (R) conditions, followed by immunoblotting with anti-PD1 (FIG. 20A), anti-SIRPα (FIG. 20B) and anti-4-1BBL (FIG. 20C) antibodies.

FIGS. 21A-C are photographs of a Western Blot analysis of TSP215, TSP215_V1, TSP214, TSP214 V1. TSP217 and DSP218. The samples presented in the figures are of crude (non-purified)-five days-supernatant. The supernatants are from Expi293F cells that were transfected with plasmids encoding to the heterodimers indicated. The control sup is of a five days-supernatant of non-transfected Expi293F cells. Supernatant samples containing approximately 50 ng of the heterodimers proteins were separated on SDS-PAGE at non-reducing (NR) or reducing (R) conditions, followed by immunoblotting with anti-4-1BBL (FIG. 21A), anti-SIRPα (FIG. 21B) and anti-LILRB2 (FIG. 21C) antibodies.

FIGS. 22A-B demonstrate binding of the PD1 arm of the TSP401 (FIG. 22A) and TSP501 (FIG. 22B) heterodimers to the ligand PDL1 expressed on the surface of DLD1 PDL1 overexpressing cell line compared to the DLD1-WT negative control cell line. Binding was determined by immuno-staining of the 4-1BBL domain using an anti-4-1BBL antibody following incubation of the cells with the indicated heterodimer, followed by flow cytometric analysis.

FIG. 23 demonstrates binding of TSP401 and TSP501 to HT1080 WT and 4-1BB overexpressing cell lines. The binding of the heterodimer proteins to the cell lines was determined following incubation of the cells with the heterodimer by immuno-staining of its PD1 domain using an anti-PD1 antibody, followed by flow cytometric analysis.

FIG. 24 demonstrates binding of TSP214 to HT1080 overexpressing 4-1BB cell line. The binding of the heterodimer to the receptor expressing cell line was determined following incubation of the cells with the heterodimer by immuno-staining of its LILRB2 domain using an anti-LILRB2 antibody, followed by flow cytometric analysis. Incubation of the cells with an anti-4-1BB blocking antibody abolished the binding of TSP214 to the cells, demonstrating specificity.

FIG. 25 demonstrates binding of TSP215 to HT1080 overexpressing 4-1BB cell line. The binding of the heterodimer to the 4-1BB and CD47 expressing cell line was determined following incubation of the cells with the heterodimer by immuno-staining of its LILRB2 domain using an anti-LILRB2 antibody, followed by flow cytometric analysis. For specificity testing, binding was determined in the absence or presence of an anti-CD47 blocking antibody, anti 4-1BB blocking antibody or a combination of both blocking antibodies.

FIGS. 26A-B demonstrate binding of the PD1-SIRPα-sc3xCD40 heterodimer TSP112 to CD40 expressed on the surface of cells. FIG. 26A is a histogram demonstrating expression of the CD40 receptor on HT1080-CD40 overexpressing cell line, as determined using an anti-CD40 antibody, followed by flow cytometric analysis. FIG. 26B demonstrates binding of TSP112 to HT1080 overexpressing CD40 cells. The binding of the heterodimer to the CD40 expressing cell line was determined following incubation of the cells with the heterodimer by immuno-staining of its PD1 domain using an anti-PD1 antibody, followed by flow cytometric analysis.

FIGS. 27A-C demonstrate binding of the PD1-TIGIT-sc3x4-1BBL heterodimer TSP501 to CD155 (PVR) expressed on the surface of cells. FIGS. 26A-B show histograms demonstrating expression of endogenous CD155 on DLD1-WT cells and no expression on U937 cells, as determined using an anti-CD155 antibody, followed by flow cytometric analysis. FIG. 27C demonstrates binding of TSP501 to DLD1-WT expressing cells and no binding to U937 cells. Binding was determined following incubation of the cells with the heterodimer by immuno-staining of its 4-1BBL domain using an anti-4-1BBL antibody, followed by flow cytometric analysis.

FIGS. 28A-C demonstrate simultaneous binding of DSP214 and TSP215 heterodimers to their respective counterparts. FIGS. 28A-B demonstrates binding of DSP214 (FIG. 28A) and TSP215 (FIG. 28B) to HLA-G and 41BB. FIG. 28C demonstrate binding of DSP215 to CD47 and 41BB. Supernatants containing the heterodimer TSP214 or control supernatant (FIG. 28A) or purified TSP215 heterodimer (FIGS. 28B-C) were incubated in HLA-G, CD47 or BSA pre-coated 96-wells plates. Binding was detected by incubation with 41BB-biotin, followed by streptavidin-HRP and TMB substrate according to standard ELISA protocol using a plate reader at 450 nm, with reference at 620 nm.

FIGS. 29A-B demonstrate activation of 41BB-mediated signal transduction by the heterodimers TSP401 and TSP501. FIG. 29A presents IL8 secretion from HT1080 4-1BB cells incubated in PDL1 and CD24 coated plates in the presence of supernatants containing TSP401. FIG. 29B presents IL-8 secretion from HT1080 4-1BB cells incubated in PDL1 and CD155 (PVR) coated plates in the presence of supernatants containing TSP501.

FIGS. 30A-B demonstrate activation of CD40-mediated signal transduction by the heterodimers TSP112, TSP217 and DSP218. FIG. 30A presents IL8 secretion from HT1080 CD40 cells incubated in PDL1 and CD47 coated plates in the presence of supernatants containing TSP112. FIG. 30B presents IL8 secretion from HT1080 CD40 cells incubated in HLA-G and CD47 coated plates in the presence of supernatants containing TSP217 or DSP218.

FIG. 31 demonstrates activation of 41BB-mediated signal transduction by the heterodimer TSP215. CHO-K1-CD47 were co-cultured with HT1080-41BB cells in presence of serial dilutions of supernatant containing TSP215.

FIG. 32 shows schematic representations of compositions and arrangements of heterodimers contemplated by some embodiments of the invention.

### DESCRIPTION OF DETAILED EMBODIMENTS OF THE INVENTION

The present invention, in some embodiments thereof, relates to heterodimers and methods of use thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Dual Signaling Proteins (DSP), also known as Signal-Converting-Proteins (SCP), are bi-functional fusion proteins that link an extracellular portion of a type I membrane protein (extracellular amino-terminus), to an extracellular portion of a type II membrane protein (extracellular carboxyl-terminus), forming a fusion protein with two active sides.

Whilst reducing the present invention to practice, the present inventors have now generated heterodimers comprising an extracellular portion of a type I membrane protein and an extracellular portion of a type II membrane protein.

Thus, according to an aspect of the present invention, there is provided a heterodimer comprising a dimerizing moiety attached to at least one amino acid sequence of at least one type I membrane protein capable of at least binding a natural ligand or receptor of said at least one type I membrane protein and to at least one amino acid sequence of at least one type II membrane protein capable of at least binding a natural ligand or receptor of said at least one type II membrane protein.

As used herein, the term “heterodimer” refers to a non-naturally occurring dimeric protein formed by the artificial attachment of two different proteins (referred to herein as monomers).

According to specific embodiments, the monomers of the heterodimer are not covalently attached.

According to other specific embodiments, the monomers of the heterodimer are covalently attached.

According to other specific embodiments, the monomers of the heterodimer are attached by a disulfide bond.

According to specific embodiments, the monomers of the heterodimer are attached by disulfide bonds.

As used herein the term “dimerizing moiety” refers to a moiety capable of attaching two different monomers to form a heterodimer. Such dimerizing moieties are known in the art and include chemical and proteinaceous moieties.

The dimerizing moiety is attached to the at least one amino acid sequence of at least one type I membrane protein and to the at least one amino acid sequence of at least one type II membrane protein.

According to specific embodiments, the dimerizing moiety is directly attached to the amino acid sequence of the type I membrane protein and/or the type II membrane protein.

According to specific embodiments, the dimerizing moiety is non-directly attached to the amino acid sequence of the type I membrane protein and/or the type II membrane protein.

According to specific embodiments, the dimerizing moiety is covalently attached to the amino acid sequence of the type I membrane protein and/or the type II membrane protein.

According to specific embodiments, the dimerizing moiety is non-covalently attached to the amino acid sequence of the type I membrane protein and/or the type II membrane protein.

According to specific embodiments, the dimerizing moiety is heterologous to the type I membrane protein and/or the type II membrane protein.

According to specific embodiments, the dimerizing moiety is a composition of at least two different molecules.

According to specific embodiments, the dimerizing moiety is a non-proteinaceous moiety, e.g. a cross linker, an organic polymer, a synthetic polymer, a small molecule and the like.

Numerous such non-proteinaceous moieties are known in the art and can be commercially obtained from e.g. Santa Cruz, Sigma-Aldrich, Proteochem and the like. According to specific embodiments, the non-proteinaceous moiety is a heterobifunctional cross linker. Heterobifunctional cross linkers have two different reactive ends. Typically, in the first step, a monomer is modified with one reactive group of the heterobifunctional reagent; the remaining free reagent is removed. In the second step, the modified monomer is mixed with a second monomer, which is then allowed to react with modifier group at the other end of the reagent. The most widely used couple proteins through amine and sulfhydryl groups (the least stable amine reactive NHS-esters couple first and after removal of uncoupled reagent, the coupling to the sulfhydryl group proceeds). The sulfhydryl reactive groups are generally maleimides, pyridyl disulfides and alpha-halocetyls. Other crosslinkers include carbodiimides, which link between carboxyl groups (—COOH) and primary amines (—NH2). Another approach is to modify the lysine residues of one monomer to thiols and the second monomer is modified by addition of maleimide groups followed by formation of stable thioester bonds between the monomers. If one of the monomers has native thiols, these groups can be reacted directly with maleimide attached to the other monomer. There are also heterobifunational cross-linkers with one phororeactive end, such as Bis[2-(4-azidosalicylamido)ethyl)] disulfide, BASED. Photoreactive groups are used when no specific groups are available to react with—as photoreactive groups react non-specifically upon exposure to UV light. Non-limiting Examples of such heterobifunctional cross linkers include, but are not limited to: Alkyne-PEG4-maleimide, Alkyne-PEG5-N-hydroxysuccinimidyl ester, Maleimide-PEG-succinimidyl ester, Azido-PEG4-phenyloxadiazole methylsulfone, LC-SMCC (succinimidyl-4-(N-maleimidomethyl)cyclohexane-1-carboxy-(6-amidocaproate)), MPBH (4-(4-N-Maleimidophenyl)butyric acid hydrazide hydrochloride+1/2 dioxane), PDPH (3-(2-pyridyldithio)propionyl hydrazide), SIAB (N-succinimidyl (4-iodoacetyl)aminobenzoate), SMPH (succinimidyl-6-((b-maleimidopropionamido)hexanoate), Sulfo-KMUS (N-(κ-maleimidoundecanoyloxy) sulfosuccinimide ester), Sulfo-SIAB (sulfosuccinimidyl (4-iodoacetyl)aminobenzoate), 3-(Maleimido)propionic acid N-hydroxysuccinimide ester, Methoxycarbonylsulfenyl chloride, Propargyl-PEG-acid, Amino-PEG-t-butyl ester, BocNH-PEG5-acid, BMPH (N-(β-maleimidopropionic acid) hydrazide, trifluoroacetic acid salt), ANB-NOS, BMPS, EMCS, GMBS, LC-SPDP, MBS, SBA, SIA, Sulfo-SIA, SMCC, SMPB, SMPH, SPDP, Sulfo-LC-SPDP, Sulfo-MBS, Sulfo-SANPAH, Sulfo-SMCC.

According to other specific embodiments, the dimerizing moiety is a proteinaceous moiety. According to specific embodiments, the dimerizing moiety comprises members of affinity pairs polypeptide having two distinct affinity moieties for two different affinity complementary tags. Such affinity pairs are well known in the art and include, but are not limited to hemagglutinin (HA), anti-HA, AviTag™, V5, Myc, T7, FLAG, HSV, VSV-G, His, biotin, avidin, streptavidin, rhizavedin, metal affinity tags, lectins affinity tags. The skilled artisan would know which tag to select.

According to specific embodiments, the dimerizing moiety is an Fc domain of an antibody (e.g., of IgG, IgA, IgD or IgE) or a fragment thereof.

According to specific embodiments, the dimerizing moiety is an Fc domain of human IgG4.

According to specific embodiments, the dimerizing moiety is an Fc domain of human IgG1.

According to specific embodiments, the dimerizing moiety is an Fc domain monomer.

According to other specific embodiments, the dimerizing moiety is an Fc domain dimer.

There are a number of mechanisms that can be used to generate a heterodimer using an Fc domain of an antibody, such as, but not limited to, knob-into-hole or charge pairs (see e.g. Gunasekaran et al., J. Biol. Chem. 285(25):19637 (2010), hereby incorporated by reference in its entirety).

Thus, according to specific embodiments, the Fc domain may comprise conservative and non-conservative amino acid substitutions (also referred to herein as mutations).

When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG. [Amino acid substitution matrices from protein blocks. Proc. Natl. Acad. Sci. U.S.A. 1992, 89(22): 10915-9].

Additional description on conservative amino acid and non-conservative amino acid substitutions is further provided hereinbelow.

Such substitution in an Fc domain are known in the art.

A representative example, which can be used with specific embodiments of the invention is the “knob-into-hole” (“KIH”) form. Such knob and hole mutations are well known in the art and disclosed e.g. in U.S. Pat. No. 8,216,805, Shane Atwell et Al. J. Mol. Biol. (1997) 270, 26-35; Cater et al. (Protein Engineering vol. 9 no. 7 pp. 617-621, 1996); and A. Margaret Merchant et. al. Nature Biotechnology (1998) 16 July, the contents of which are fully incorporated herein by reference. In addition, as described in Merchant et al., Nature Biotech. 16:677 (1998), these “knobs and hole” mutations can be combined with disulfide bonds to skew formation to heterodimerization.

Thus, according to specific embodiments, one of the monomers comprises an Fc domain comprising a knob mutation(s) and the other monomer comprises an Fc domain comprising a hole mutation(s).

It is within the scope of those skilled in the art to select a specific immunoglobulin Fc domain from particular immunoglobulin classes and subclasses and to select a first Fc variant for knob mutation and the other for hole mutation. Non-limiting Examples of substitutions that can be used with specific embodiments include S228P, L235E, T366W, Y349C, T366S, L368A, Y407V and/or E356C (according to EU numbering (Kabat, E. A., T. T. Wu, M. Reid-Miller, H. M. Perry and K. S. Gottesman. 1987. Sequences of proteins of Immunological Interest. US. Dept. of Health and Human Services, Bethesda) corresponding to the human IgG4 amino acid sequence set forth in SEQ ID NO: 109, 110 or 111, or L234A, L235A, Y349C, T366W, T354C, D356C, T366S, L368A and/or Y407V (according to EU numbering (Kabat, E. A., T. T. Wu, M. Reid-Miller, H. M. Perry and K. S. Gottesman. 1987. Sequences of proteins of Immunological Interest. US. Dept. of Health and Human Services, Bethesda) corresponding to the human IgG1 amino acid sequence set forth in SEQ ID NO: 12, 13 or 14.

According to specific embodiments, the Fc domain comprises an amino acid sequence selected from the group consisting of SEQ ID NO: 109-114.

According to a specific embodiments, the monomer comprising the amino acid sequence of the type I membrane protein comprises a knob mutation(s).

According to a specific embodiments, the monomer comprising the amino acid sequence of the type I membrane protein comprises a hole mutation(s).

According to a specific embodiments, the monomer comprising the amino acid sequence of the type II membrane protein comprises a knob mutation(s).

According to a specific embodiments, the monomer comprising the amino acid sequence of the type II membrane protein comprises a hole mutation(s).

According to a specific embodiment, a monomer comprising an amino acid sequence of the type I membrane protein and an amino acid sequence of the type II membrane protein comprises a knob mutation(s).

According to a specific embodiment, the monomer comprising an amino acid sequence of the type I membrane protein and an amino acid sequence of the type II membrane protein comprises a hole mutation(s).

According to specific embodiments, the dimerizing moiety comprises a leucine zipper or a helix-loop-helix.

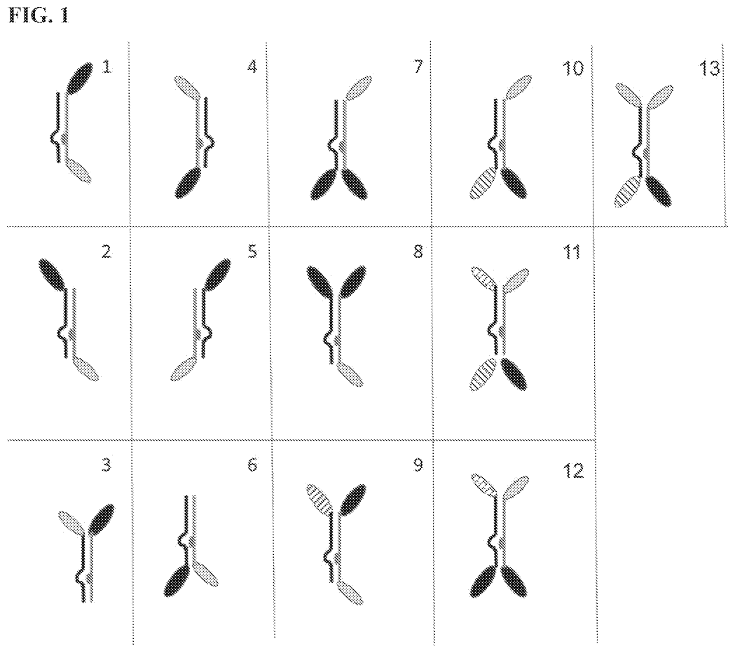

The heterodimer of some embodiments comprises at least one amino acid sequence of at least one type I membrane protein and at least one amino acid sequence of at least one type II membrane protein. Non-limiting examples of possible arrangements of such a heterodimer is schematically shown in FIG. 1.

According to specific embodiments, the heterodimer arrangement is selected from the arrangements shown in panels 1-13 of FIG. 1, each possibility represents a separate embodiment of the present invention.

According to specific embodiments, each of the monomers comprised in the heterodimer comprises an amino acid sequence of a type I membrane protein and/or an amino acid sequence of a type TT membrane protein.

According to specific embodiments, each of the monomers comprised in the heterodimer comprises an amino acids sequence of a type I membrane protein and an amino acid sequence of a type II membrane protein.

According to specific embodiments, the heterodimer comprises a first monomer comprising an amino acid sequence of a type II membrane protein and a second monomer comprising an amino acid sequence of a type I membrane protein.

According to specific embodiments, the heterodimer comprises a first monomer comprising an amino acid sequence of a type I membrane protein and an amino acid sequence of a type II membrane protein.

According to specific embodiments, the heterodimer comprises a first monomer comprising an amino acid sequence of a type I membrane protein and an amino acid sequence of a type II membrane protein and a second monomer comprising an amino acid sequence of a type I membrane protein.

When both monomers comprise an amino acid sequence of a type I membrane protein, the type I membrane protein amino acid sequence may be identical, may be of the same type I membrane protein but of a different sequence or may be of different type I membrane proteins.

Thus, according to specific embodiments, the amino acid sequence of the type I membrane protein of the first monomer is identical to the amino acid sequence of the type I membrane protein of the second monomer.

According to other specific embodiments, the amino acid sequence of the type I membrane protein of the first monomer is distinct (i.e. different) from the amino acid sequence of the type I membrane protein of the second monomer.

According to specific embodiments, the type I membrane protein of the first monomer is distinct (i.e. different) from the type I membrane protein of the second monomer.

When both monomers comprise an amino acid sequence of a type II membrane protein, the type II membrane protein amino acid sequence may be identical, may be of the same type II membrane protein but of a different sequence or may be of different type II membrane proteins.

Thus, according to specific embodiments, the amino acid sequence of the type II membrane protein of the first monomer is identical to the amino acid sequence of the type II membrane protein of the second monomer.

According to other specific embodiments, the amino acid sequence of the type II membrane protein of the first monomer is distinct (i.e. different) from the amino acid sequence of the type II membrane protein of the second monomer.

According to specific embodiments, the type II membrane protein of the first monomer is distinct (i.e. different) from the type II membrane protein of the second monomer.

According to specific embodiments, when the dimerizing moiety is a proteinaceous moiety the amino acid sequence of the type I membrane protein is attached to an N-terminus of the proteinaceous dimerizing moiety and the amino acid sequence of the type II membrane protein is attached to a C-terminus of the proteinaceous dimerizing moiety.

According to specific embodiments, when the dimerizing moiety is a proteinaceous moiety the amino acid sequence of the type I membrane protein is attached to a C-terminus of the proteinaceous dimerizing moiety and the amino acid sequence of the type II membrane protein is attached to an N-terminus of the proteinaceous dimerizing moiety.

According to specific embodiments, when the dimerizing moiety is a proteinaceous dimer moiety both the amino acid sequence of the type I membrane protein and the amino acid sequence of the type II membrane protein are attached to C-termini or N-termini of the proteinaceous dimer dimerizing moiety.

According to specific embodiments, when the dimerizing moiety is a proteinaceous dimer moiety both the amino acid sequence of the type I membrane protein and the amino acid sequence of the type II membrane protein are attached to C-termini of the proteinaceous dimer dimerizing moiety.

According to specific embodiments, when the dimerizing moiety is a proteinaceous dimer moiety both the amino acid sequence of the type I membrane protein and the amino acid sequence of the type II membrane protein are attached to N-termini of the proteinaceous dimer dimerizing moiety.

As used herein, the phrase “an amino acid sequence of a type I membrane protein” refers to a contiguous amino acids sequence of a type I membrane protein capable of at least binding the native ligand or receptor of the type I membrane protein.

According to specific embodiments, such an amino acid sequence comprises an extracellular domain of the type I membrane protein or a functional fragment thereof.

As used herein, the phrase “type I membrane protein” refers to a transmembrane protein having an N-terminus extracellular domain.

Non-limiting examples of such Type I membrane proteins include PD1, SIRPα, LAG3, BTN3A1, CD27, CD80, CD86, ENG, NLGN4X, CD84, TIGIT, CD40, IL-8, IL-10, CD164, LY6G6F, CD28, CTLA4, BTLA, LILRB1, LILRB2, TYROBP, ICOS, VEGFA, CSF1, CSF1R, VEGFB, BMP2, BMP3, GDNF, PDGFC, PDGFD, RAETIE, CD155, CD166, MICA, NRG1, HVEM, DR3, TEK, TGFB1, LY96, CD96, KIT, CD244 GFER and SIGLEC.

According to specific embodiments, the type I membrane protein is selected from the group consisting of PD1, SIRPα, LAG3, BTN3A1, CD27, CD80, CD86, ENG, NLGN4X, CD84, TIGIT, CD40, IL-8, IL-10, CD164, LY6G6F, CD28, CTLA4, BTLA, LILRB1, LILRB2, TYROBP, ICOS, VEGFA, CSF1, CSF1R, VEGFB, BMP2, BMP3, GDNF, PDGFC, PDGFD, RAETIE, CD155, CD166, MICA, NRG1, HVEM, DR3, TEK, TGFB1, LY96, CD96, KIT, CD244, and GFER.

According to specific embodiments, the type I membrane protein is selected from the group consisting of PD1, SIRPα, LAG3, TIGIT, LILRB1, LILRB2, CSF1, CSF1R and TGFB1.

According to specific embodiments, the type I membrane protein is selected from the group consisting of PD1, SIRPα, TIGIT, LILRB2 and SIGLEC.

According to specific embodiments, the Type I membrane protein is an immune modulator.

As used herein the term “immune modulator” refers to a protein that modulates an immune cell response (i.e. activation or function). Immune modulators can positively regulate immune cell activation or function or negatively regulate immune cell activation or function. Such immune modulators are known in the art and include an immune-check point protein, a cytokine and the like.

According to specific embodiments, the immune modulator is an immune activator.

According to other specific embodiments, the immune modulator is an immune suppressor or inhibitor.

Non-limiting examples of Type I membrane protein immune modulators include, but are not limited to PD1, SIRPα, CD28, CSF1R, IL-8, IL-10, CTLA4, ICOS, CD27, CD80, CD86, SIGLEC10 and TIGIT. According to specific embodiments, the type I membrane protein comprises a single type I membrane protein.

According to specific embodiments, the type I membrane protein comprises at least one type I membrane protein.

According to specific embodiments, the type I membrane protein comprises at least two type I membrane proteins.

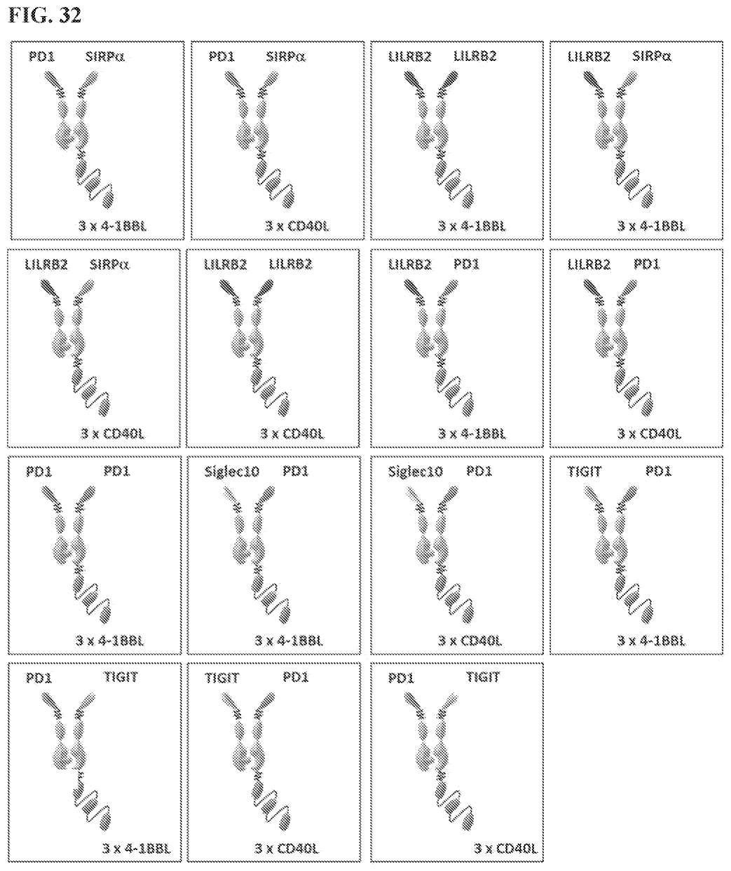

According to specific embodiments, the heterodimer composition and arrangement is selected from the heterodimers schematically shown in FIG. 32, each possibility represents a separate embodiment of the present invention.

According to specific embodiments, the type I membrane protein is selected from the group consisting of PD1 and SIRPα.

According to specific embodiments, the type I membrane protein is PD1.

As used herein the term “PD1 (Programmed Death 1, also known as CD279)” refers to the polypeptide of the PDCD1 gene (Gene ID 5133) or a functional homolog e.g., functional fragment thereof. According to specific embodiments, the term “PD1” refers to a functional homolog of PD1 polypeptide. According to specific embodiments, PD1 is human PD1. According to a specific embodiment, the PD1 protein refers to the human protein, such as provided in the following GenBank Number NP_005009.

Two ligands for PD-1 have been identified so far, PDL1 and PDL2 (also known as B7-DC).

According to a specific embodiment, the PDL1 protein refers to the human protein, such as provided in the following GenBank Number NP_001254635 and NP_054862. According to a specific embodiment, the PDL2 protein refers to the human protein, such as provided in the following GenBank Number NP_079515.

According to specific embodiments, PD1 amino acid sequence comprises SEQ ID NO: 1.

According to specific embodiments, PD1 amino acid sequence consists of SEQ ID NO: 1.

As use herein, the phrase “a functional homolog of the polypeptide of the PDCD1 gene” or “a functional fragment of the polypeptide of the PDCD1 gene” refers to a portion of the polypeptide, a functional homologue (naturally occurring or synthetically/recombinantly produced) and/or a PD1 polypeptide comprising conservative and non-conservative amino acid substitutions, which maintains at least the activity of the full length PD1 of binding PD-L1 and/or PD-L2.

Assays for testing binding are well known in the art and include, but not limited to flow cytometry, BiaCore, bio-layer interferometry Blitz® assay, HPLC.

According to specific embodiments, the PD1 binds PD-L1 with a Kd of 1 nM-100 PM, 10-nM-10 μM, 100 nM-100 μM, 200 nM-10 μM, as determined by SPR analysis, each possibility represents a separate embodiment of the present invention.

According to specific embodiments, the PD1 binds PDL1 with a Kd of about 270 nM as determined by SPR analysis.

According to specific embodiments, the PD1 binds PDL1 with a Kd of about 8-9 μM as determined by SPR analysis.

According to specific embodiments, the PD1 comprises an extracellular domain of said PD1 or a functional fragment thereof.

According to specific embodiments, PD1 amino acid sequence comprises SEQ ID NO: 5, 6, or 7.

According to specific embodiments, PD1 amino acid sequence consists of SEQ ID NO: 5, 6 or 7.

The term “PD1” also encompasses functional homologues (naturally occurring or synthetically/recombinantly produced), which exhibit the desired activity (i.e., binding PD-L1 and/or PD-L2). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 1, 5, 6, or 7; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

As used herein, “identity” or “sequence identity” refers to global identity, i.e., an identity over the entire amino acid or nucleic acid sequences disclosed herein and not over portions thereof.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the PD1 polypeptide may comprise conservative and non-conservative amino acid substitutions. Such substitution are known in the art and disclosed e.g. in Maute et al. PNAS, 2015 Nov. 24; 112(47):E6506-14; Ju Yeon et al. Nature Communications 2016 volume 7, Article number: 13354 (DOI: 10.1038/ncomms13354); and Zack K M et al. Structure. 2015 23(12): 2341-2348 (DOI:10.1016/j.str.2015.09.010), the contents of which are fully incorporated herein by reference.

According to specific embodiments, one or more amino acid mutations are located at an amino acid residue selected from: V39, L40, N41, Y43, R44, M45, S48, N49, Q50, T51, D52, K53, A56, Q63, G65, Q66, V72, H82, M83, R90, Y96, L97, A100, S102, L103, A104, P105, K106, and A107 corresponding to the PD1 amino acid sequence set forth in SEQ ID NO: 6. According to specific embodiments, one or more amino acid mutations are located at an amino acid residue selected from: V39, L40, N41, Y43, R44, M45, S48, N49, Q50, T51, D52, K53, A56, Q63, G65, Q66, C68, V72, H82, M83, R90, Y96, L97, A100, S102, L103, A104, P105, K106, and A107 corresponding to the PD1 amino acid sequence set forth in SEQ ID NO: 6.

According to specific embodiments, one or more amino acid changes are selected from the group consisting of: (1) V39H or V39R; (2) L40V or L40I; (3) N41I or N41V; (4) Y43F or Y43H; (5) R44Y or R44L; (6) M45Q, M45E, M45L, or M45D; (7) S48D, S48L, S48N, S48G, or S48V; (8) N49C, N49G, N49Y, or N49S; (9) Q50K, Q50E, or Q50H; (10) T51V, T51L, or T51A; (11) D52F, D52R, D52Y, or D52V; (12) K53T or K53L; (13) A56S or A56L; (14) Q63T, Q63I, Q63E, Q63L, or Q63P; (15) G65N, G65R, G65I, G65L, G65F, or G65V; (16) Q66P; (17) V72I; (18) H82Q; (19) M83L or M83F; (20) R90K; (21) Y96F; (22) L97Y, L97V, or L97I; (23) A100I or A100V; (24) S102T or S102A; (25) L103I, L103Y, or L103F; (26) A104S, A104H, or A104D; (27) P105A; (28) K106G, K106E, K106I, K106V, K106R, or K106T; and (29) A107P, A107I, or A107V corresponding to the PD1 amino acid sequence set forth in SEQ ID NO: 6.

According to specific embodiments, one or more amino acid changes are selected from the group consisting of: (1) V39H or V39R; (2) L40V or L40I; (3) N41I or N41V; (4) Y43F or Y43H; (5) R44Y or R44L; (6) M45Q, M45E, M45L, or M45D; (7) S48D, S48L, S48N, S48G, or S48V; (8) N49C, N49G, N49Y, or N49S; (9) Q50K, Q50E, or Q50H; (10) T51V, T51L, or T51A; (11) D52F, D52R, D52Y, or D52V; (12) K53T or K53L; (13) A56S or A56L; (14) Q63T, Q63I, Q63E, Q63L, or Q63P; (15) G65N, G65R, G65I, G65L, G65F, or G65V; (16) Q66P; (17) C68S (18), V72I; (19) H82Q; (20) M83L or M83F; (21) R90K; (22) Y96F; (23) L97Y, L97V, or L97I; (24) A100I or A100V; (25) S102T or S102A; (26) L103I, L103Y, or L103F; (27) A104S, A104H, or A104D; (28) P105A; (29) K106G, K106E, K106I, K106V, K106R, or K106T; and (30) A107P, A107I, or A107V corresponding to the PD1 amino acid sequence set forth in SEQ ID NO: 6.

According to specific embodiments, an amino acid mutation is located at an amino acid residue C93 corresponding to the PD1 amino acid sequence set forth in SEQ ID NO: 1 (e.g. equivalent to an amino acid residue C68 corresponding to the PD1 amino acid sequence set forth in SEQ ID NO: 6).

According to specific embodiments, the PD1 polypeptide may comprise a C to S amino acid modification in a position corresponding to amino acid residue 93 of the PD1 amino acid sequence set forth in SEQ ID NO: 1 (e.g. equivalent to amino acid residue 68 of the PD1 amino acid sequence set forth in SEQ ID NO: 6).

Thus, according to specific embodiments, the PD1 amino acid sequence comprises SEQ ID NO: 3.

According to specific embodiments, PD1 amino acid sequence consists of SEQ ID NO: 3.

As used herein, the phrase “corresponding to PD1 amino acid sequence as set forth in SEQ ID NO: 1”, “corresponding to SEQ ID NO: 1”, “corresponding to PD1 amino acid sequence as set forth in SEQ ID NO: 6” or “corresponding to SEQ ID NO: 6”, intends to include the corresponding amino acid residue relative to any other PD1 amino acid sequence.

Additional description on conservative amino acid and non-conservative amino acid substitutions is further provided hereinabove and below.

The PD1 of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 3, 5, 6, 7, 9, 11, 13, 15, 17, 19, 21, 23, 25, 27, 29, 31, 33, 35, 37, 39, 41, 43 or 45; or at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same, each possibility represents a separate embodiment of the present invention.

According to specific embodiments, the PD1 amino acid sequence does not comprise any of amino acid segments P1-L5 and/or F146-V150 corresponding to SEQ ID NO: 7.

According to specific embodiments, the PD1 amino acid sequence does not comprise any of amino acid residues P1-L5 and/or F146-V150 corresponding to SEQ ID NO: 7.

According to specific embodiments, PD1 amino acid sequence comprises 100-288 amino acids, 100-200 amino acids, 120-180 amino acids, 120-160, 130-170 amino acids, 130-160, 130-150, 140-160 amino acids, 145-155 amino acids, 123-166 amino acids, 138-145 amino acids, 123-148 amino acids, 126-148 amino acids, 123-140 amino acids, 126-140 amino acids, 127-140 amino acids, 130-140 amino acids, each possibility represents a separate embodiment of the present invention.

According to specific embodiments, the PD1 amino acid sequence comprises an amino acid sequence selected from the group consisting of SEQ ID NO: 3, 5, 6, 7, 9, 11, 13, 15, 17, 19, 21, 23, 25, 27, 29, 31, 33, 35, 37, 39, 41, 43 and 45.

According to specific embodiments, the PD1 amino acid sequence consists of an amino acid sequence selected from the group consisting of SEQ ID NO: 3, 5, 6, 7, 9, 11, 13, 15, 17, 19, 21, 23, 25, 27, 29, 31, 33, 35, 37, 39, 41, 43 and 45.

According to specific embodiments, the PD1 amino acid sequence comprises an amino acid sequence selected from the group consisting of SEQ ID NO: 13 and 7.

According to specific embodiments, the PD1 amino acid sequence consists of an amino acid sequence selected from the group consisting of SEQ ID NO: 13 and 7.

According to specific embodiments, the PD1 nucleic acid sequence encoding the PD1 amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 2, 4, 8, 10, 12, 14, 16, 18, 20, 22, 24, 26, 28, 30, 32, 34, 36, 38, 40, 42, 44 or 46, each possibility represents a separate embodiment of the present invention.

According to specific embodiments, the PD1 nucleic acid sequence encoding the PD1 amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 14 or 8.

According to specific embodiments, the PD1 nucleic acid sequence encoding the PD1 amino acid sequence comprises a nucleic acid sequence selected from the group consisting of SEQ ID NO: 2, 4, 8, 10, 12, 14, 16, 18, 20, 22, 24, 26, 28, 30, 32, 34, 36, 38, 40, 42, 44 and 46.

According to specific embodiments, the PD1 nucleic acid sequence encoding the PD1 amino acid sequence consists of a nucleic acid sequence selected from the group consisting of SEQ ID NO: 2, 4, 8, 10, 12, 14, 16, 18, 20, 22, 24, 26, 28, 30, 32, 34, 36, 38, 40, 42, 44 and 46.

According to specific embodiments, the PD1 nucleic acid sequence encoding the PD1 amino acid sequence comprises SEQ ID NO: 14 or 8.

According to specific embodiments, the PD1 nucleic acid sequence encoding the PD1 amino acid sequence consists of SEQ ID NO: 14 or 8.

According to specific embodiments, the type I membrane protein is SIRPα.

As used herein the term “SIRPα (Signal Regulatory Protein Alpha, also known as CD172a)” refers to the polypeptide of the SIRPA gene (Gene ID 140885) or a functional homolog e.g., functional fragment thereof. According to specific embodiments, the term “SIRPα” refers to a functional homolog of SIRPα polypeptide. According to specific embodiments, SIRPα is human SIRPα. According to a specific embodiment, the SIRPα protein refers to the human protein, such as provided in the following GenBank Number NP_001035111, NP_001035112, NP_001317657 or NP_542970.

According to specific embodiments, SIRPα amino acid sequence comprises SEQ ID NO: 69.

According to specific embodiments, SIRPα amino acid sequence consists of SEQ ID NO: 69.

As use herein, the phrase “functional homolog of the polypeptide of the SIRPA gene” or “functional fragment of the polypeptide of the SIRP1 gene” refers to a portion of the polypeptide, a functional homologue (naturally occurring or synthetically/recombinantly produced) and/or a SIRPα polypeptide comprising conservative and non-conservative amino acid substitutions, which maintains at least the activity of the full length SIRPα of binding CD47. Assays for testing binding are well known in the art and are further described hereinabove and below.

According to a specific embodiment, the CD47 protein refers to the human protein, such as provided in the following GenBank Numbers NP_001768 or NP_942088.

According to specific embodiments, the SIRPα binds CD47 with a Kd of 0.1-100 μM, 0.1-10 μM, 1-10 μM, 0.1-5 μM, or 1-2 μM as determined by SPR, each possibility represents a separate embodiment of the present invention.

According to specific embodiments, the SIRPα comprises an extracellular domain of said SIRPα or a functional fragment thereof.

According to specific embodiments, SIRPα amino acid sequence comprises SEQ ID NO: 71.

According to specific embodiments, SIRPα amino acid sequence consists of SEQ ID NO: 71.

The term “SIRPα” also encompasses functional homologues (naturally occurring or synthetically/recombinantly produced), which exhibit the desired activity (i.e., binding CD47).

Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 69 or 71; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the SIRPα polypeptide may comprise conservative and non-conservative amino acid substitutions. Such substitutions are known in the art and disclosed e.g. in Weiskopf K et al. Science. (2013); 341(6141):88-91, the contents of which are fully incorporated herein by reference.

According to specific embodiments, one or more amino acid mutations are located at an amino acid residue selected from: L4, V6, A21, A27, I31, E47, K53, E54, H56, V63, L66, K68, V92 and F96 corresponding to the SIRPα amino acid sequence set forth in SEQ ID NO: 71.

According to specific embodiments, the SIRPα amino acid sequence comprises a mutation at an amino acid residue selected from the group consisting of L4, A27, E47 and V92 corresponding to the SIRPα amino acid sequence set forth in SEQ ID NO: 71.

According to specific embodiments, one or more amino acid mutations are selected from the group consisting of: L4V or L4I, V6I or V6L, A21V, A27I or A27L, I31F or I31T, E47V or E47L, K53R, E54Q, H56P or H56R, V63I, L66T or L66G, K68R, V92I and F94L or F94V corresponding to the SIRPα amino acid sequence set forth in SEQ ID NO: 71.

According to specific embodiments, the SIRPα amino acid sequence comprises a mutation selected from the group consisting of L4I, A27I, E47V and V92I corresponding to the SIRPα amino acid sequence set forth in SEQ ID NO: 71.

As used herein, the phrase “corresponding to the SIRPα amino acid sequence set forth in SEQ ID NO: 71” or “corresponding to SEQ ID NO: 71” intends to include the corresponding amino acid residue relative to any other SIRPα amino acid sequence.

According to specific embodiments, the SIRPα amino acid sequence comprises SEQ ID NO: 75.

According to specific embodiments, the SIRPα amino acid sequence consists of SEQ ID NO: 75.

Additional description on conservative amino acid and non-conservative amino acid substitutions is further provided hereinabove and below.

The SIRP amino acid sequence of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 71, 73, 75 or 77; or at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same, each possibility represents a separate embodiment of the present invention.

According to specific embodiments, the SIRPα amino acid sequence does not comprise the amino acid segment K117-Y343 corresponding to SEQ ID NO: 71.

According to specific embodiments, the SIRPα amino acid sequence does not comprise any of amino acid residues K117-Y343 corresponding to SEQ ID NO: 71.

According to specific embodiments, SIRPα amino acid sequence comprises 100-504, 100-500 amino acids, 150-450 amino acids, 200-400 amino acids, 250-400 amino acids, 300-400 amino acids, 320-420 amino acids, 340-350 amino acids, 300-400 amino acids, 340-450 amino acids, 100-200 amino acids, 100-150 amino acids, 100-125 amino acids, 100-120 amino acids, 100-119 amino acids, 105-119 amino acids, 110-119 amino acids, 115-119 amino acids, 105-118 amino acids, 110-118 amino acids, 115-118 amino acids, 105-117 amino acids, 110-117 amino acids, 115-117 amino acids, each possibility represents a separate embodiment of the present invention.

According to specific embodiments, the SIRPα amino acid sequence comprises an amino acid sequence selected from the group consisting of SEQ ID NO: 71, 73, 75 and 77.

According to specific embodiments, the SIRPα amino acid sequence comprises SEQ ID NO: 71.

According to specific embodiments, the SIRPα amino acid sequence consists of SEQ ID NO: 71.

According to specific embodiments, a nucleic acid sequence encoding the SIRPα amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 72, 74, 76 or 78, each possibility represents a separate embodiment of the present invention.

According to specific embodiments, a nucleic acid sequence encoding the SIRPα amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 72, each possibility represents a separate embodiment of the present invention.

According to specific embodiments, the nucleic acid sequence encoding the SIRPα amino acid sequence comprises SEQ ID NO: 72.

According to specific embodiments, the nucleic acid sequence encoding the SIRPα amino acid sequence consists of SEQ ID NO: 72.

According to specific embodiments, the type I membrane protein is TIGIT.

As used herein the term “TIGIT (I Cell Immunoreceptor With Ig And ITIM Domains)” refers to the polypeptide of the TIGIT gene (Gene ID 201633) or a functional homolog e.g., functional fragment thereof. According to specific embodiments, the term “TIGIT” refers to a functional homolog of TIGIT polypeptide. According to specific embodiments, TIGIT is human TIGIT. According to a specific embodiment, the TIGIT protein refers to the human protein, such as provided in the following GenBank Number NP_776160 or XP_024309156.

According to specific embodiments, TIGIT amino acid sequence comprises SEQ ID NO: 160.

According to specific embodiments, TIGIT amino acid sequence consists of SEQ ID NO: 160.

As use herein, the phrase “functional homolog of the polypeptide of the TIGIT gene” or “functional fragment of the polypeptide of the TIGIT gene” refers to a portion of the polypeptide, a functional homologue (naturally occurring or synthetically/recombinantly produced) and/or a TIGIT polypeptide comprising conservative and non-conservative amino acid substitutions, which maintains at least the activity of the full length TIGIT of binding CD155 (PVR).

Assays for testing binding are well known in the art and are further described hereinabove and below.

According to a specific embodiment, the CD155 protein refers to the human protein, such as provided in the following GenBank Numbers NP_001129240, NP_001129241, NP_001129242, NP_006496.

According to specific embodiments, the TIGIT binds CD155 with a Kd of 0.01-100 μM, 0.1-100 μM, 0.1-10 μM or 0.1-5 μM as determined by SPR, each possibility represents a separate embodiment of the present invention.

According to specific embodiments, the TIGIT comprises an extracellular domain of said TIGIT or a functional fragment thereof.

According to specific embodiments, TIGIT amino acid sequence comprises SEQ ID NO: 164.

According to specific embodiments, TIGIT amino acid sequence consists of SEQ ID NO: 164.

According to specific embodiments, TIGIT amino acid sequence comprises SEQ ID NO: 130.

According to specific embodiments, TIGIT amino acid sequence consists of SEQ ID NO: 130.

The term “TIGIT” also encompasses functional homologues (naturally occurring or synthetically/recombinantly produced), which exhibit the desired activity (i.e., binding CD155).

Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 160, 164 or 130; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the TIGIT polypeptide may comprise conservative and non-conservative amino acid substitutions.

According to specific embodiments, one or more amino acid mutations are located at an amino acid residue selected from: 142 and C69 corresponding to the TIGIT amino acid sequence set forth in SEQ ID NO: 160.

According to specific embodiments, one or more amino acid mutations are selected from the group consisting of: I42A and C69S corresponding to the TIGIT amino acid sequence set forth in SEQ ID NO: 160.

As used herein, the phrase “corresponding to the TIGIT amino acid sequence set forth in SEQ ID NO: 160” or “corresponding to SEQ ID NO: 160” intends to include the corresponding amino acid residue relative to any other TIGIT amino acid sequence.

According to specific embodiments, the TIGIT amino acid sequence comprises SEQ ID NO: 132.

According to specific embodiments, the TIGIT amino acid sequence consists of SEQ ID NO: 132.

Additional description on conservative amino acid and non-conservative amino acid substitutions is further provided hereinabove and below.

According to specific embodiments, TIGIT amino acid sequence comprises 100-244 amino acids, 100-200 amino acids, 100-150 amino acids, 120-140 amino acids, each possibility represents a separate embodiment of the present invention.

According to specific embodiments, a nucleic acid sequence encoding the TIGIT amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 131 or 133.

According to specific embodiments, the nucleic acid sequence encoding the TIGIT amino acid sequence comprises SEQ ID NO: 133.

According to specific embodiments, the nucleic acid sequence encoding the TIGIT amino acid sequence consists of SEQ ID NO: 133.

According to specific embodiments, the type I membrane protein is LILRB2.

As used herein the term “LILRB2 (Leukocyte immunoglobulin-like receptor subfamily B member 2)” refers to the polypeptide of the LILRB2 gene (Gene ID 10288) or a functional homolog e.g., functional fragment thereof. According to specific embodiments, the term “LILRB2” refers to a functional homolog of LIRB2 polypeptide. According to specific embodiments, LILRB2 is human LILRB2. According to a specific embodiment, the LILRB2 protein refers to the human protein, such as provided in the following GenBank Number NP_001074447, NP_001265332, NP_001265333, NP_001265334, NP_001265335.

According to specific embodiments, LILRB2 amino acid sequence comprises SEQ ID NO: 161.

According to specific embodiments, LILRB2 amino acid sequence consists of SEQ ID NO: 161.

As use herein, the phrase “functional homolog of the polypeptide of the LILRB2 gene” or “functional fragment of the polypeptide of the LILRB2 gene” refers to a portion of the polypeptide, a functional homologue (naturally occurring or synthetically/recombinantly produced) and/or a LILRB2 polypeptide comprising conservative and non-conservative amino acid substitutions, which maintains at least the activity of the full length LILRB2 of binding a major histocompatibility molecule (MHC, e.g. HLA-G).

Assays for testing binding are well known in the art and are further described hereinabove and below.

According to specific embodiments, the LILRB2 binds MHC (e.g. HLA-G) with a Kd of 0.1 nM-100 PM, 0.1 nM-10 μM, 1 nM-1 μM, 1-100 nM, or 1-10 nM as determined by SPR, each possibility represents a separate embodiment of the present invention.

According to specific embodiments, the LILRB2 comprises an extracellular domain of said LILRB2 or a functional fragment thereof.

According to specific embodiments, the LILRB2 amino acid sequence comprises SEQ ID NO: 165.

According to specific embodiments, the LILRB2 amino acid sequence consists of SEQ ID NO: 165.

The extracellular domain of LILRB2 comprises 4 Ig-like domains, known as D1-D4.

Hence, according to specific embodiments, the amino acid sequence of LILRB2 comprises at least one Ig-like domain.

According to specific embodiments, the amino acid sequence of LILRB2 comprises at least two Ig-like domains, at least three Ig-like domains or four Ig-like domains.

According to specific embodiments, the amino acid sequence of LILRB2 comprises domains D1 and D2 of LILRB2; domains D1, D2 and D3 of LILRB2, domains D1, D2 and D4 or LILRB2, or domains D1, D2, D3 and D4 of LILRB2.

According to specific embodiments, LILRB2 amino acid sequence comprises SEQ ID NO: 115 or 117.

According to specific embodiments, LILRB2 amino acid sequence consists of SEQ ID NO: 115 or 117.

The term “LILRB2” also encompasses functional homologues (naturally occurring or synthetically/recombinantly produced), which exhibit the desired activity (i.e., binding MHC, e.g. HLA-G). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 161, 165, 115 or 117; or at least 70%, at least 75%, at least 80 10%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the LILRB2 polypeptide may comprise conservative and non-conservative amino acid substitutions.

Additional description on conservative amino acid and non-conservative amino acid substitutions is further provided hereinabove and below.

According to specific embodiments, LILRB2 amino acid sequence comprises 100-597 amino acids, 100-500 amino acids, 100-400 amino acids, 150-400 amino acids, 300-400 amino acids, 350-400 amino acids, 150-250 amino acids, each possibility represents a separate embodiment of the present invention.

According to specific embodiments, a nucleic acid sequence encoding the LILRB2 amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 116 or 118.

According to specific embodiments, the nucleic acid sequence encoding the LILRB2 amino acid sequence comprises SEQ ID NO: 116.

According to specific embodiments, the nucleic acid sequence encoding the LILRB2 amino acid sequence consists of SEQ ID NO: 118.

According to specific embodiments, the type I membrane protein is SIGLEC.

As used herein the term “SIGLEC (Sialic acid-binding immunoglobulin-type lectins)” refers to the polypeptide encoded by a SIGLEC gene or a functional homolog e.g., functional fragment thereof. According to specific embodiments, the term “SIGLEC” refers to a functional homolog of SIGLEC polypeptide.

As use herein, the phrase “functional homolog of the polypeptide of a SIGLEC gene” or “functional fragment of the polypeptide of a SIGLEC gene” refers to a portion of the polypeptide, a functional homologue (naturally occurring or synthetically/recombinantly produced) and/or a SIGLEC polypeptide comprising conservative and non-conservative amino acid substitutions, which maintains at least the activity of the full length SIGLEC of binding sialic acid, and more specifically sialic acid-containing carbohydrates (sialoglycans).

According to specific embodiments, the SIGLEC comprises an extracellular domain of the SIGLEC or a functional fragment thereof.

The extracellular domain of SIGLEC comprises Ig-like domains.

Hence, according to specific embodiments, the amino acid sequence of SIGLEC comprises at least one Ig-like domain.

According to specific embodiments, SIGLEC is human SIGLEC.

Non-limiting examples of SIGLECs include SIGLEC-1, SIGLEC-2, SIGLEC-3, SIGLEC-4, SIGLEC-5, SIGLEC-6, SIGLEC-7, SIGLEC-8, SIGLEC-9, SIGLEC-10, SIGLEC-11, SIGLEC-12, SIGLEC-13, SIGLEC-14, SIGLEC-15, SIGLEC-16, SIGLEC-17. According to specific embodiments, the SIGLEC is selected from the group consisting of SIGLEC-2, SIGLEC-3, SIGLEC-4, SIGLEC-7, SIGLEC-9, SIGLEC-10, SIGLEC-12 and SIGLEC-15, each possibility represents a separate embodiment of the present invention.

According to a specific embodiment, the SIGLEC is SIGLEC-10.

As used herein the term “SIGLEC-10 (Sialic acid-binding Ig-like lectin 10)” refers to the polypeptide of the SIGLEC10 gene (Gene ID 89790) or a functional homolog e.g., functional fragment thereof. According to a specific embodiment, the SIGLEC10 protein refers to the human protein, such as provided in the following GenBank Number NP_001164627, NP_001164628, NP_001164629, NP_001164630, NP_001164632.

According to specific embodiments, SIGLEC10 amino acid sequence comprises SEQ ID NO: 162.

According to specific embodiments, SIGLEC amino acid sequence consists of SEQ ID NO: 162.

As use herein, the phrase “functional homolog of the polypeptide of the SIGLEC10 gene” or “functional fragment of the polypeptide of the SIGLEC10 gene” refers to a portion of the polypeptide, a functional homologue (naturally occurring or synthetically/recombinantly produced) and/or a SIGLEC-10 polypeptide comprising conservative and non-conservative amino acid substitutions, which maintains at least the activity of the full length SIGLEC-10 of binding sialic acid expressed on CD24 and/or CD52.

Assays for testing binding are well known in the art and are further described hereinabove and below.

According to specific embodiments, the SIGLEC10 binds CD24 or CD52 with a Kd of 1 nM-100 μM, 0.01-100 μM, 0.01-10 μM, 0.1-10 μM, 0.1-5 μM, or 0.1-1 μM as determined by SPR, each possibility represents a separate embodiment of the present invention.

According to specific embodiments, the SIGLEC-10 comprises an extracellular domain of said SIGLEC-10 or a functional fragment thereof.

According to specific embodiments, the amino acid sequence of SIGLEC-10 comprises at least one Ig-like domain.

According to specific embodiments, the amino acid sequence of SIGLEC-10 comprises at least two Ig-like domain.

According to specific embodiments, SIGLEC-10 amino acid sequence comprises SEQ ID NO: 129.

According to specific embodiments, SIGLEC-10 amino acid sequence comprises SEQ ID NO: 125.

According to specific embodiments, SIGLEC-10 amino acid sequence consists of SEQ ID NO: 125.

The term “SIGLEC-10” also encompasses functional homologues (naturally occurring or synthetically/recombinantly produced), which exhibit the desired activity (i.e., binding sialic acid expressed on CD24 and/or CD52). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 162, 129 or 125; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the SIGLEC-10 polypeptide may comprise conservative and non-conservative amino acid substitutions.

According to specific embodiments, one mutation is located at an amino acid residue C36 corresponding to the SIGLEC-10 amino acid sequence set forth in SEQ ID NO: 162.

According to specific embodiments, one amino acid mutation is C36S corresponding to the SIGLEC-10 amino acid sequence set forth in SEQ ID NO: 162.

As used herein, the phrase “corresponding to the SIGLEC-10 amino acid sequence set forth in SEQ ID NO: 162” or “corresponding to SEQ ID NO: 162” intends to include the corresponding amino acid residue relative to any other SIGLEC-10 amino acid sequence.

According to specific embodiments, the SIGLEC-10 amino acid sequence comprises SEQ ID NO: 127.

According to specific embodiments, the SIGLEC-10 amino acid sequence consists of SEQ ID NO: 127.

Additional description on conservative amino acid and non-conservative amino acid substitutions is further provided hereinabove and below.

According to specific embodiments, SIGLEC-10 amino acid sequence comprises 100-639 amino acids, 100-600 amino acids, 100-550 amino acids, 100-300 amino acids, 100-200 amino acids, 100-150 amino acids, each possibility represents a separate embodiment of the present invention.

According to specific embodiments, a nucleic acid sequence encoding the SIGLEC-10 amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 126 or 128.

According to specific embodiments, the nucleic acid sequence encoding the SIGLEC-10 amino acid sequence comprises SEQ ID NO: 128.

According to specific embodiments, the nucleic acid sequence encoding the SIGLEC-10 amino acid sequence consists of SEQ ID NO: 128.

As used herein, the phrase “an amino acid sequence of a type II membrane protein” refers to a contiguous amino acids sequence of a type II membrane protein capable of at least binding the native ligand or receptor of the type II membrane protein.

According to specific embodiments, such an amino acid sequence comprises an extracellular domain of the type II membrane protein or a functional fragment thereof.

As used herein, the phrase “type II membrane protein” refers to a transmembrane protein having a C-terminus extracellular domain.

Non-limiting examples of such Type II membrane proteins include 4-1BBL, FasL, TRAIL, TNF-alpha, TNF-beta, OX40L, CD40L, CD27L, CD30L, RANKL, TWEAK, APRIL, BAFF, LIGHT, VEGI, GITRL, EDA1/2, Lymphotoxin alpha and Lymphotoxin beta.

According to specific embodiments, the type II membrane protein is selected from the group consisting of 4-1BBL, OX40L, CD40L, LIGHT and GITRL.

According to specific embodiments, the Type II membrane protein is an immune modulator.

Such immune modulator include, but are not limited to 4-1BBL, TNF-alpha, TNF-beta, OX40L, CD40L, CD27L and CD30L.

According to specific embodiments, the type II membrane protein comprises a single type II membrane protein.

According to specific embodiments, the type II membrane protein comprises at least one type II membrane protein.

According to specific embodiments, the type II membrane protein comprises at least two type II membrane proteins.

According to specific embodiments, the Type II membrane protein is 4-1BBL.

As used herein the term “4-1BBL (also known as CD137L and TNFSF9)” refers to the polypeptide of the TNFSF9 gene (Gene ID 8744) or a functional homolog e.g., functional fragment thereof. According to specific embodiments, the term “4-1BBL” refers to a functional homolog of 4-1BBL polypeptide. According to specific embodiments, 4-1BBL is human 4-1BBL.

According to a specific embodiment, the 4-1BBL protein refers to the human protein, such as provided in the following GenBank Number NP_003802.

According to specific embodiments, 4-1BBL amino acid sequence comprises SEQ ID NO: 47.

According to specific embodiments, 4-1BBL amino acid sequence consists of SEQ ID NO: 47.

As use herein, the phrase “functional homolog of a polypeptide of the TNFSF9 gene” or “functional fragment of a polypeptide of the TNFSF9 gene” refers to a portion of the polypeptide, a functional homologue (naturally occurring or synthetically/recombinantly produced) and/or a 4-1BBL polypeptide comprising conservative and non-conservative amino acid substitutions, which maintains at least one of the activities of the full length 4-1BBL e.g., (i) binding 4-1BB, (ii) activating 4-1BB signaling pathway, (iii) activating immune cells expressing 4-1BB, (iv) forming a homotrimer.

According to specific embodiments, the functional 4-1BBL homolog or fragment is capable of at least (i).

According to specific embodiments, the functional 4-1BBL homolog or fragment is capable of (i)+(ii), (i)+(iii), (i)+(iv), (i)+(ii)+(iii), (i)+(ii)+(iv), (i)+(iii)+(iv), (ii)+(iii)+(iv) or (i)+(ii)+(iii)+(iv).

According to a specific embodiment, the 4-1BB protein refers to the human protein, such as provided in the following GenBank Number NP_001552.

Assays for testing binding are well known in the art and are further described hereinabove and below.

According to specific embodiments, the 4-1BBL binds 4-1BB with a Kd of about 0.1-1000 nM, 0.1-100 nM, 1-100 nM, or 55.2 nM as determined by SPR, each possibility represents a separate embodiment of the claimed invention.

Methods of determining trimerization are well known in the art and include, but are not limited to NATIVE-PAGE, SEC-HPLC 2D gels, gel filtration, SEC-MALS, Analytical ultracentrifugation (AUC) Mass spectrometry (MS), capillary gel electrophoresis (CGE).

As used herein the terms “activating” or “activation” refer to the process of stimulating an immune cell (e.g. T cell, B cell, NK cell, phagocytic cell) that results in cellular proliferation, maturation, cytokine production, phagocytosis and/or induction of regulatory or effector functions.

According to specific embodiments, activating comprises co-stimulating.

As used herein the term “co-stimulating” or “co-stimulation” refers to transmitting a secondary antigen independent stimulatory signal (e.g. 4-1BB signal) resulting in activation of the immune cell.

According to specific embodiments, activating comprises suppressing an inhibitory signal (e.g. PDL1 signal) resulting in activation of the immune cell.

Methods of determining signaling of a stimulatory or inhibitory signal are well known in the art and also disclosed in the Examples section which follows, and include, but are not limited to, binding assay using e.g. BiaCore, HPLC or flow cytometry, enzymatic activity assays such as kinase activity assays, and expression of molecules involved in the signaling cascade using e.g.

PCR, Western blot, immunoprecipitation and immunohistochemistry. Additionally or alternatively, determining transmission of a signal (co-stimulatory or inhibitory) can be effected by evaluating immune cell activation or function. Methods of evaluating immune cell activation or function are well known in the art and include, but are not limited to, proliferation assays such as CFSE staining, MTS, Alamar blue, BRDU and thymidine incorporation, cytotoxicity assays such as CFSE staining, chromium release, Calcin AM, cytokine secretion assays such as intracellular cytokine staining, ELISPOT and ELISA, expression of activation markers such as CD25, CD69, CD137, CD107a, PD1, and CD62L using flow cytometry.

According to specific embodiments, determining the signaling activity or activation is effected in-vitro or ex-vivo e.g. in a mixed lymphocyte reaction (MLR), as further described hereinbelow. For the same culture conditions the signaling activity or the immune cell activation or function are generally expressed in comparison to the signaling, activation or function in a cell of the same species but not contacted with the heterodimer, a polynucleotide encoding same or a host cell encoding same; or contacted with a vehicle control, also referred to as control.

According to specific embodiments, the 4-1BBL comprises an extracellular domain of said 4-1BBL or a functional fragment thereof.

According to specific embodiments, 4-1BBL amino acid sequence comprises SEQ ID NO: 49.

According to specific embodiments, 4-1BBL amino acid sequence consists of SEQ ID NO: 49.

The term “4-1BBL” also encompasses functional homologues (naturally occurring or synthetically/recombinantly produced), which exhibit the desired activity (as defined hereinabove). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 47 or 49; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the 4-1BBL polypeptide may comprise conservative amino acid substitutions, as further described hereinabove and below.

According to specific embodiments, the 4-1BBL amino acid sequence does not comprise the amino acid segment A1-V6, A1-G14 or A1-E23 corresponding to SEQ ID NO: 49.

According to specific embodiments, the 4-1BBL amino acid sequence does not comprise any of amino acid residues A1-V6 or A1-G14 or A1-E23 corresponding to SEQ ID NO: 49.

According to specific embodiments, the 4-1BBL amino acid sequence does not comprise the amino acid segment G198-E205 corresponding to SEQ ID NO: 49.

According to specific embodiments, the 4-1BBL amino acid sequence does not comprise any of amino acid residues G198-E205 corresponding to SEQ ID NO: 49.

As used herein, the phrase “corresponding to SEQ ID NO: 49” intends to include the corresponding amino acid residue relative to any other 4-1BBL amino acid sequence.

According to specific embodiments, 4-1BBL amino acid sequence comprises 100-254 amino acids, 150-250 amino acids, 100-250 amino acids, 150-220 amino acids, 180-220 amino acids, 180-210 amino acids, 185-205 amino acids, 185-200 amino acids, 185-199 amino acids, 170-197 amino acids, 170-182 amino acids, 190-210 amino acids, each possibility represents a separate embodiment of the present invention.

The 4-1BBL of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 49, 51, 53, 558, 57, 59, 61, 63 or 65; or at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same, each possibility represents a separate embodiment of the present invention.

According to specific embodiments, the 4-1BBL amino acid sequence comprises an amino acid sequence selected from the group consisting of SEQ ID NO: 49, 51, 53, 55, 57, 59, 61, 63 and 65.

According to specific embodiments, the 4-1BBL amino acid sequence consists of an amino acid sequence selected from the group consisting of SEQ ID NO: 49, 51, 53, 55, 57, 59, 61, 63 and 65.

According to specific embodiments, the nucleic acid sequence encoding the 4-1BBL amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 50, 52, 54, 56, 58, 60, 62, 64 and 66, each possibility represents a separate embodiment of the present invention.

According to specific embodiments, the nucleic acid sequence encoding the 4-1BBL amino acid sequence comprises a nucleic acid sequence selected from the group consisting of SEQ ID NO: 50, 52, 54, 56, 58, 60, 62, 64 and 66.

According to specific embodiments, the nucleic acid sequence encoding the 4-1BBL amino acid sequence consists of a nucleic acid sequence selected from the group consisting of SEQ ID NO: 50, 52, 54, 56, 58, 60, 62, 64 and 66.

According to specific embodiments, the Type II membrane protein is CD40L.

As used herein the term “CD40L (also known as CD154)” refers to the polypeptide of the CD40LG gene (Gene ID 959) or a functional homolog e.g., functional fragment thereof. According to specific embodiments, the term “CD40L” refers to a functional homolog of CD40L polypeptide. According to specific embodiments, CD40L is human CD40L. According to a specific embodiment, the CD40L protein refers to the human protein, such as provided in the following GenBank Number NP_000065.

According to specific embodiments, CD40L amino acid sequence comprises SEQ ID NO: 163.

According to specific embodiments, CD40L amino acid sequence consists of SEQ ID NO: 163.

As use herein, the phrase “functional homolog of a polypeptide of the CD40LG gene” or “functional fragment of a polypeptide of the CD40LG gene” refers to a portion of the polypeptide, a functional homologue (naturally occurring or synthetically/recombinantly produced) and/or a CD40L polypeptide comprising conservative and non-conservative amino acid substitutions, which maintains at least one of the activities of the full length CD40L e.g., (i) binding CD40, (ii) activating CD40 signaling pathway, (iii) activating immune cells expressing CD40, (iv) forming a homotrimer.

According to specific embodiments, the functional CD40L homolog or fragment is capable of at least (i).

According to specific embodiments, the functional CD40L homolog or fragment is capable of (i)+(ii), (i)+(iii), (i)+(iv), (i)+(ii)+(iii), (i)+(ii)+(iv), (i)+(iii)+(iv), (ii)+(iii)+(iv) or (i)+(ii)+(iii)+(iv).

According to a specific embodiment, the CD40 protein refers to the human protein, such as provided in the following GenBank Number NP_001241, NP_001289682, NP_001309350, NP_001309351, NP_690593.

Assays for testing binding, trimerization, activation, co-stimulation and signaling are well known in the art and are further described hereinabove and below.

According to specific embodiments, the CD40L binds CD40 with a Kd of about 0.1-1000 nM, 0.1-100 nM, 1-100 nM, or 1-5 nM as determined by SPR, each possibility represents a separate embodiment of the claimed invention.

According to specific embodiments, the CD40L comprises an extracellular domain of said CD40L or a functional fragment thereof.

According to specific embodiments, CD40L amino acid sequence comprises SEQ ID NO: 122.

According to specific embodiments, CD40L amino acid sequence consists of SEQ ID NO: 122.

According to specific embodiments, CD40L amino acid sequence comprises SEQ ID NO: 123. According to specific embodiments, CD40L amino acid sequence consists of SEQ ID NO: 123.

The term “CD40L” also encompasses functional homologues (naturally occurring or synthetically/recombinantly produced), which exhibit the desired activity (as defined hereinabove). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 163, 122 or 123; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the CD40L polypeptide may comprise conservative amino acid substitutions, as further described hereinabove and below.

According to specific embodiments, one mutation is located at an amino acid residue C194 corresponding to the CD40L amino acid sequence set forth in SEQ ID NO: 163.

According to specific embodiments, on mutation is C194S corresponding to the CD40L amino acid sequence set forth in SEQ ID NO: 163.

As used herein, the phrase “corresponding to the CD40L amino acid sequence set forth in SEQ ID NO: 163” or “corresponding to SEQ ID NO: 163” intends to include the corresponding amino acid residue relative to any other CD40L amino acid sequence.

According to specific embodiments, the CD40L amino acid sequence comprises SEQ ID NO: 119.

According to specific embodiments, the CD40L amino acid sequence consists of SEQ ID NO: 119.

Additional description on conservative amino acid and non-conservative amino acid substitutions is further provided hereinabove and below.

According to specific embodiments, CD40L amino acid sequence comprises 100-261 amino acids, 100-220 amino acids, 100-200 amino acids, 120-160 amino acids, each possibility represents a separate embodiment of the present invention.

According to specific embodiments, a nucleic acid sequence encoding the CD40L amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 120 or 124.

According to specific embodiments, the nucleic acid sequence encoding the CD40L amino acid sequence comprises SEQ ID NO: 120.

According to specific embodiments, the nucleic acid sequence encoding the CD40L amino acid sequence consists of SEQ ID NO: 120.

According to specific embodiments, the amino acid sequence of a type II membrane protein comprised in the heterodimer disclosed herein comprises three repeats of a type II membrane protein (e.g. 4-1BBL, CD40L) amino acid sequence.

According to specific embodiments, each of the three repeats is capable of at least binding a native ligand or receptor of the type II membrane protein.

According to specific embodiments, the three repeats have an identical type II membrane protein (e.g. 4-1BBL, CD40L) amino acid sequence.

According to other specific embodiments, the three repeats are distinct, i.e. have different type II membrane protein (e.g. 4-1BBL, CD40L) amino acid sequences.

According to other specific embodiments, two of the three repeats have an identical type II membrane protein (e.g. 4-1BBL, CD40L) amino acid sequence.

According to specific embodiments, the type II membrane protein amino acid sequence does not comprise a linker between each of said three repeats of said type II membrane protein amino acid sequence.

According to other specific embodiments, the type II membrane protein amino acid sequence comprises a linker between each of said three repeats of said type II membrane protein amino acid sequence. Any linker known in the art can be used with specific embodiments of the invention.

Non-limiting examples of linkers that can be used are described in details hereinbelow.

According to a specific embodiment, the linker is a (GGGGS)x2+GGGG (SEQ ID NO: 96) linker.

According to a specific embodiment, the linker is a GGGGSGGGG (SEQ ID NO: 97) linker.

According to a specific embodiment, the linker is a GGGGSx3 (SEQ ID NO: 134) linker.

Thus, for example, according to specific embodiments, the 4-1BBL amino acid sequence comprised in the heterodimer comprises three repeats of a 4-1BBL amino acid sequence.

According to specific embodiments, each of the three repeats is capable of at least one of: (i) binding 4-1BB, (ii) activating 4-1BB signaling pathway, (iii) activating immune cells expressing 4-1BB, (iv) forming a homotrimer.

According to specific embodiments, the repeated sequence can be any of the 4-1BBL as defined herein.

According to specific embodiments, at least one of the repeats comprises a 4-1BBL amino acid sequence disclosed herein.

According to specific embodiments, at least one of the repeats consists of a 4-1BBL amino acid sequence disclosed herein.

According to specific embodiments, the 4-1BBL amino acid sequence comprises three repeats of an amino acid sequence comprising SEQ ID NO: 51.

According to specific embodiments, the 4-1BBL amino acid sequence comprises three repeats of an amino acid sequence consisting of SEQ ID NO: 51.

Thus, according to specific embodiments, the 4-1BBL amino acid sequence comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 67.

According to specific embodiments, the 4-1BBL amino acid sequence comprises SEQ ID NO: 67.

According to specific embodiments, the 4-1BBL amino acid sequence consists of SEQ ID NO: 67.

According to specific embodiments, a nucleic acid sequence encoding the 4-1BBL amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 68.

According to specific embodiments, the 4-1BBL nucleic acid sequence comprises SEQ ID NO: 68.

According to specific embodiments, the 4-1BBL nucleic acid sequence consists of SEQ ID NO: 68.

As another example, according to specific embodiments, the CD40L amino acid sequence comprised in the heterodimer comprises three repeats of a CD40L amino acid sequence.

According to specific embodiments, each of the three repeats is capable of at least one of: (i) binding CD40, (ii) activating CD40 signaling pathway, (iii) activating immune cells expressing CD40, (iv) forming a homotrimer.

According to specific embodiments, the repeated sequence can be any of the CD40L as defined herein.

According to specific embodiments, at least one of the repeats comprises a CD40L amino acid sequence disclosed herein.

According to specific embodiments, at least one of the repeats consists of a CD40L amino acid sequence disclosed herein.

According to specific embodiments, the CD40L amino acid sequence comprises three repeats of an amino acid sequence comprising SEQ ID NO: 119.

According to specific embodiments, the CD40L amino acid sequence comprises three repeats of an amino acid sequence consisting of SEQ ID NO: 119.

Thus, according to specific embodiments, the CD40L amino acid sequence comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 121.

According to specific embodiments, the CD40L amino acid sequence comprises SEQ ID NO: 121.

According to specific embodiments, the CD40L amino acid sequence consists of SEQ ID NO: 121.

According to specific embodiments, a nucleic acid sequence encoding the CD40L amino acid sequence has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 166.

According to specific embodiments, the CD40L nucleic acid sequence comprises SEQ ID NO: 166.

According to specific embodiments, the CD40L nucleic acid sequence consists of SEQ ID NO: 166.

According to specific embodiments, the type I membrane protein is PD1, the type II membrane protein is 4-1BBL, and the heterodimer comprises a first monomer comprising an amino acid sequence of PD1 and an amino acid sequence of 4-1BBL and a second monomer comprising an amino acid sequence of PD1.

According to specific embodiments, the amino acid of the PD1 of the first monomer and the amino acid of the PD1 of the second monomer are identical.

According to specific embodiments, the amino acid of the PD1 of the first monomer and the amino acid of the PD1 of the second monomer are distinct (i.e. different).

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 79 and 81; or SEQ ID NO: 79 and 83.

According to specific embodiments, the heterodimer comprises SEQ ID NO: 79 and 81; or SEQ ID NO: 79 and 83.

According to specific embodiments, the heterodimer consists of SEQ ID NO: 79 and 81; or SEQ ID NO: 79 and 83.

According to specific embodiments, the type I membrane protein is LILRB2, the type II membrane protein is 4-1BBL, and the heterodimer comprises a first monomer comprising an amino acid sequence of LILRB2 and an amino acid sequence of 4-1BBL and a second monomer comprising an amino acid sequence of LILRB2.

According to specific embodiments, the amino acid of the LILRB2 of the first monomer and the amino acid of the LILRB2 of the second monomer are identical.

According to specific embodiments, the amino acid of the LILRB2 of the first monomer and the amino acid of the LILRB2 of the second monomer are distinct (i.e. different).

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 142 and 138; or SEQ ID NO: 144 and 140.

According to specific embodiments, the heterodimer comprises SEQ ID NO: 142 and 138; or SEQ ID NO: 144 and 140.

According to specific embodiments, the heterodimer consists of SEQ ID NO: 142 and 138; or SEQ ID NO: 144 and 140.

According to specific embodiments, the type I membrane protein is LILRB2, the type II membrane protein is CD40L, and the heterodimer comprises a first monomer comprising an amino acid sequence of LILRB2 and an amino acid sequence of CD40L and a second monomer comprising an amino acid sequence of LILRB2.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 148 and 138.

According to specific embodiments, the heterodimer comprises SEQ ID NO: 148 and 138.

According to specific embodiments, the heterodimer consists of SEQ ID NO: 148 and 138.

According to specific embodiments, the type I membrane protein is selected from the group consisting of PD1 and SIRPα, the type II membrane protein is 4-1BBL, and the heterodimer comprises a first monomer comprising an amino acid sequence of SIRPα and an amino acid sequence of 4-1BBL and a second monomer comprising an amino acid sequence of PD1.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 85 and 81; SEQ ID NO: 89 and 91; or SEQ ID NO: 85 and 83.

According to specific embodiments, the heterodimer comprises SEQ ID NO: 85 and 81; SEQ ID NO: 89 and 91; or SEQ ID NO: 85 and 83.

According to specific embodiments, the heterodimer consists of SEQ ID NO: 85 and 81; SEQ ID NO: 89 and 91; or SEQ ID NO: 85 and 83.

According to specific embodiments, the type I membrane protein is selected from the group consisting of PD1 and SIRPα, the type II membrane protein is CD40L, and the heterodimer comprises a first monomer comprising an amino acid sequence of SIRPα and an amino acid sequence of CD40L and a second monomer comprising an amino acid sequence of PD1.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 146 and 81.

According to specific embodiments, the heterodimer comprises SEQ ID NO: 146 and 81.

According to specific embodiments, the heterodimer consists of SEQ ID NO: 146 and 81.

According to specific embodiments, the type I membrane protein is selected from the group consisting of LILRB2 and SIRPα, the type II membrane protein is 4-1BBL, and the heterodimer comprises a first monomer comprising an amino acid sequence of SIRPα and an amino acid sequence of 4-1BBL and a second monomer comprising an amino acid sequence of LILRB2.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 85 and 138; or SEQ ID NO: 85 and 140.

According to specific embodiments, the heterodimer comprises SEQ ID NO: 85 and 138; or SEQ ID NO: 85 and 140.

According to specific embodiments, the heterodimer consists of SEQ ID NO: 85 and 138; or SEQ ID NO: 85 and 140.

According to specific embodiments, the type I membrane protein is selected from the group consisting of LILRB2 and SIRPα, the type II membrane protein is CD40L, and the heterodimer comprises a first monomer comprising an amino acid sequence of SIRPα and an amino acid sequence of CD40L and a second monomer comprising an amino acid sequence of LILRB2.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 146 and 138.

According to specific embodiments, the heterodimer comprises SEQ ID NO: 146 and 138.

According to specific embodiments, the heterodimer consists of SEQ ID NO: 146 and 138.

According to specific embodiments, the type I membrane protein is selected from the group consisting of LILRB2 and PD1, the type II membrane protein is 4-1BBL, and the heterodimer comprises a first monomer comprising an amino acid sequence of PD1 and an amino acid sequence of 4-1BBL and a second monomer comprising an amino acid sequence of LILRB2.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 79 and 138.

According to specific embodiments, the heterodimer comprises SEQ ID NO: 79 and 138.

According to specific embodiments, the heterodimer consists of SEQ ID NO: 79 and 138.

According to specific embodiments, the type I membrane protein is selected from the group consisting of LILRB2 and PD1, the type TT membrane protein is CD40L, and the heterodimer comprises a first monomer comprising an amino acid sequence of PD1 and an amino acid sequence of CD40L and a second monomer comprising an amino acid sequence of LILRB2.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 154 and 138.

According to specific embodiments, the heterodimer comprises SEQ ID NO: 154 and 138.

According to specific embodiments, the heterodimer consists of SEQ ID NO: 154 and 138.

According to specific embodiments, the type I membrane protein is selected from the group consisting of SIGLEC and PD1, the type II membrane protein is 4-1BBL, and the heterodimer comprises a first monomer comprising an amino acid sequence of PD1 and an amino acid sequence of 4-1BBL and a second monomer comprising an amino acid sequence of SIGLEC.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 79 and 150.

According to specific embodiments, the heterodimer comprises SEQ ID NO: 79 and 150.

According to specific embodiments, the heterodimer consists of SEQ ID NO: 79 and 150.

According to specific embodiments, the type I membrane protein is selected from the group consisting of SIGLEC and PD1, the type II membrane protein is CD40L, and the heterodimer comprises a first monomer comprising an amino acid sequence of PD1 and an amino acid sequence of CD40L and a second monomer comprising an amino acid sequence of SIGLEC.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 154 and 150.

According to specific embodiments, the heterodimer comprises SEQ ID NO: 154 and 150.

According to specific embodiments, the heterodimer consists of SEQ ID NO: 154 and 150.

According to specific embodiments, the type I membrane protein is selected from the group consisting of TIGIT and PD1, the type TT membrane protein is 4-1BBL, and the heterodimer comprises a first monomer comprising an amino acid sequence of PD1 and an amino acid sequence of 4-1BBL and a second monomer comprising an amino acid sequence of TIGIT.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 79 and 152.

According to specific embodiments, the heterodimer comprises SEQ ID NO: 79 and 152.

According to specific embodiments, the heterodimer consists of SEQ ID NO: 79 and 152.

According to specific embodiments, the type I membrane protein is selected from the group consisting of TIGIT and PD1, the type II membrane protein is CD40L, and the heterodimer comprises a first monomer comprising an amino acid sequence of PD1 and an amino acid sequence of CD40L and a second monomer comprising an amino acid sequence of TIGIT.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 154 and 152.

According to specific embodiments, the heterodimer comprises SEQ ID NO: 154 and 152.

According to specific embodiments, the heterodimer consists of SEQ ID NO: 154 and 152.

According to specific embodiments, the type I membrane protein is selected from the group consisting of TIGIT and PD1, the type II membrane protein is 4-1BBL, and the heterodimer comprises a first monomer comprising an amino acid sequence of TIGIT and an amino acid sequence of 4-1BBL and a second monomer comprising an amino acid sequence of PD1.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 156 and 81.

According to specific embodiments, the heterodimer comprises SEQ ID NO: 156 and 81.

According to specific embodiments, the heterodimer consists of SEQ ID NO: 156 and 81.

According to specific embodiments, the type I membrane protein is selected from the group consisting of TIGIT and PD1, the type TT membrane protein is CD40L, and the heterodimer comprises a first monomer comprising an amino acid sequence of TIGIT and an amino acid sequence of CD40L and a second monomer comprising an amino acid sequence of PD1.

According to specific embodiments, the heterodimer comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 158 and 81.

According to specific embodiments, the heterodimer comprises SEQ ID NO: 158 and 81.

According to specific embodiments, the heterodimer consists of SEQ ID NO: 158 and 81.

According to specific embodiments, the heterodimer disclosed herein is soluble (i.e., not immobilized to a synthetic or a naturally occurring surface).

According to specific embodiments, the heterodimer disclosed herein is immobilized to a synthetic or a naturally occurring surface.

According to specific embodiments, each of the moieties comprised in the heterodimer may comprise a linker, separating between the moieties, e.g. between the amino acid sequence of the type I membrane protein and the dimerizing moiety, between the amino acid sequence of the type I membrane protein and the dimerizing moiety, between the three repeats of the type II membrane protein amino acid sequence.

According to other specific embodiments, the heterodimer does not comprise a linker between the amino acid sequence of the type I membrane protein and the dimerizing moiety.

According to other specific embodiments, the heterodimer does not comprise a linker between the amino acid sequence of the type II membrane protein and the dimerizing moiety.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22(2): 153-167, Chen et al, (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et al., (2000), Protein Eng. 13(5):309-312, the entire contents of which are hereby incorporated by reference.

According to specific embodiments, the linker is a synthetic linker such as PEG.

According to specific embodiments, the linker may be functional. For example, without limitation, the linker may function to improve the folding and/or stability, improve the expression, improve the pharmacokinetics, and/or improve the bioactivity of the PD1-4-1BBL fusion protein. In another example, the linker may function to target the PD1-4-1BBL fusion protein to a particular cell type or location.

According to specific embodiments, the linker is a polypeptide.

Non-limiting examples of polypeptide linkers include linkers having the sequence LE, GGGGS (SEQ ID NO: 99), (GGGGS). (n=1-4) (SEQ ID NO: 98), GGGGSGGGG (SEQ ID NO: 97), (GGGGS)x2 (SEQ ID NO: 100), (GGGGS)x2+GGGG (SEQ ID NO: 96), (GGGGS)x3 (SEQ ID NO: 134), (GGGGS)x4 (SEQ ID NO: 135), (Gly)₈ (SEQ ID NO: 136), (Gly)₆ (SEQ ID NO: 137), (EAAAK). (n=1-3) (SEQ ID NO: 101), A(EAAAK)ₙA (n=2-5) (SEQ ID NO: 102), AEAAAKEAAAKA (SEQ ID NO: 103), A(EAAAK)₄ALEA(EAAAK)₄A (SEQ ID NO: 104), PAPAP (SEQ ID NO: 105), K ESGSVSS EQ LAQ FRS LD (SEQ ID NO: 106), EGKSSGSGSESKST (SEQ ID NO: 107), GSAGSAAGSGEF (SEQ ID NO: 108), and (XP)., with X designating any amino acid, e.g., Ala, Lys, or Glu.

According to specific embodiments, the linker is selected from the group consisting of GGGGS (SEQ ID NO: 99), (GGGGS). (n=1-4) (SEQ ID NO: 98), GGGGSGGGG (SEQ ID NO: 97), (GGGGS)x2 (SEQ ID NO: 100), (GGGGS)x2+GGGG (SEQ ID NO: 96), (GGGGS)x2 (SEQ ID NO: 100), (GGGGS)x3 (SEQ ID NO: 134) and (GGGGS)x4 (SEQ ID NO: 135).

According to specific embodiments, the linker is selected from the group consisting of GGGGS (SEQ ID NO: 99), (GGGGS). (n=1-4) (SEQ ID NO: 98), GGGGSGGGG (SEQ ID NO: 97), (GGGGS)x2 (SEQ ID NO: 100), (GGGGS)x2+GGGG (SEQ ID NO: 96).

According to a specific embodiment, the linker is (GGGGS)x2+GGGG (SEQ ID NO: 96).

According to a specific embodiment, the linker is (GGGGS)x2 (SEQ ID NO: 100).

According to a specific embodiment, the linker is (GGGGS)x3 (SEQ ID NO: 134).

According to a specific embodiment, the linker is (GGGGS)x4 (SEQ ID NO: 135).

According to specific embodiments, the linker is at a length of one to six amino acids.

According to specific embodiments, the linker is substantially comprised of glycine and/or serine residues (e.g. about 30%, or about 40%, or about 50%, or about 60%, or about 70%, or about 80%, or about 90%, or about 95%, or about 97% or 100% glycines and serines).

According to specific embodiments, the linker is a single amino acid linker.

In some embodiments of the invention, the one amino acid is glycine.

According to specific embodiments, the linker is not an Fc domain or a hinge region of an antibody or a fragment thereof.

According to specific embodiments, the production yield of the heterodimer is at least 1.5 fold, at least 2 fold, at least 2.5 fold, at least 3 fold, at least 5 fold higher than the production yield of a homodimer comprising the same amino acid sequences of the Type I membrane protein and Type II membrane proteins or of isolated monomers comprising same.

According to specific embodiments, the amount of aggregates of the heterodimer is at least 20%, at least 30%, at least 40%, at least 50%, at least 60%, at least 70%, at least 80%, at least 90% or at least 95% lower than the amount of aggregates of a homodimer comprising the same amino acid sequences of the Type I membrane protein and Type II membrane proteins or of isolated monomers comprising same.

According to specific embodiments, the stability of the heterodimer is at least 1.5 fold, at least 2 fold, at least 2.5 fold, at least 3 fold, at least 5 fold higher than the stability of a homodimer comprising the same amino acid sequences of the Type I membrane protein and Type II membrane proteins or of isolated monomers comprising same.

According to specific embodiments, the activity of the heterodimer is at least 1.5 fold, at least 2 fold, at least 2.5 fold, at least 3 fold, at least 5 fold higher than the activity of a homodimer comprising the same amino acid sequences of the Type I membrane protein and Type II membrane proteins or of isolated monomers comprising same.

According to specific embodiments, the safety of the heterodimer is at least 1.5 fold, at least 2 fold, at least 2.5 fold, at least 3 fold, at least 5 fold higher than the safety of a homodimer comprising the same amino acid sequences of the Type I membrane protein and Type II membrane proteins or of isolated monomers comprising same.

As the heterodimer of some embodiments of present invention comprises an amino acid sequence of a type I membrane protein and/or an amino acid sequence of a type II membrane protein which is an immune modulator, the heterodimer may be used in method of modulating immune cells, in-vitro, ex-vivo and/or in-vivo.

Thus, according to an aspect of the present invention, there is provided a method of modulating activity of immune cells, the method comprising in-vitro activating immune cells in the presence of the heterodimer, a nucleic acid construct or system encoding same or a host cell comprising same.

According to other specific embodiments, the modulating is inhibiting.

According to specific embodiments, the modulating is activating.

According to specific embodiments, the immune cells express a ligand or a receptor of said type I membrane protein or said type II membrane protein (e.g. 4-1BB).

According to specific embodiments, the immune cells comprise peripheral mononuclear blood cells (PBMCs).

As used herein the term “peripheral mononuclear blood cells (PBMCs)” refers to a blood cell having a single nucleus and includes lymphocytes, monocytes and dendritic cells (DCs).

According to specific embodiments, the PBMCs are selected from the group consisting of dendritic cells (DCs), T cells, B cells, NK cells and NKT cells.

According to specific embodiments, the PBMCs comprise T cells, B cells, NK cells and NKT cells.

Methods of obtaining PBMCs are well known in the art, such as drawing whole blood from a subject and collection in a container containing an anti-coagulant (e.g. heparin or citrate); and apheresis. Following, according to specific embodiments, at least one type of PBMCs is purified from the peripheral blood. There are several methods and reagents known to those skilled in the art for purifying PBMCs from whole blood such as leukapheresis, sedimentation, density gradient centrifugation (e.g. ficoll), centrifugal elutriation, fractionation, chemical lysis of e.g. red blood cells (e.g. by ACK), selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the immune cells comprise tumor infiltrating lymphocytes.

As used herein the term “tumor infiltrating lymphocytes (TILs) refers to mononuclear white blood cells that have lest the bloodstream and migrated into a tumor.

According to specific embodiments, the TILs are selected from the group consisting of T cells, B cells, NK cells and monocytes.

Methods of obtaining TILs are well known in the art, such as obtaining tumor samples from a subject by e.g. biopsy or necropsy and preparing a single cell suspension thereof. The single cell suspension can be obtained in any suitable manner, e.g., mechanically (disaggregating the tumor using, e.g., a GentleMACS™ Dissociator, Miltenyi Biotec, Auburn, Calif.) or enzymatically (e.g., collagenase or DNase). Following, the at least one type of TILs can be purified from the cell suspension. There are several methods and reagents known to those skilled in the art for purifying the desired type of TILs, such as selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the immune cells comprise phagocytic cells.

As used herein, the term “phagocytic cells” refer to a cell that is capable of phagocytosis and include both professional and non-professional phagocytic cells. Methods of analyzing phagocytosis are well known in the art and include for examples killing assays, flow cytometry and/or microscopic evaluation (live cell imaging, fluorescence microscopy, confocal microscopy, electron microscopy). According to specific embodiments, the phagocytic cells are selected from the group consisting of monocytes, dendritic cells (DCs) and granulocytes.

According to specific embodiments, the phagocytes comprise granulocytes.

According to specific embodiments, the phagocytes comprise monocytes.

According to specific embodiments, the immune cells comprise monocytes.

According to specific embodiments, the term “monocytes” refers to both circulating monocytes and to macrophages (also referred to as mononuclear phagocytes) present in a tissue.

According to specific embodiments, the monocytes comprise macrophages. Typically, cell surface phenotype of macrophages include CD14, CD40, CD11b, CD64, F4/80 (mice)/EMR1 (human), lysozyme M, MAC-1/MAC-3 and CD68.

According to specific embodiments, the monocytes comprise circulating monocytes. Typically, cell surface phenotypes of circulating monocytes include CD14 and CD16 (e.g. CD14++CD16-, CD14+CD16++, CD14++CD16+).

According to specific embodiments, the immune cells comprise DCs

As used herein the term “dendritic cells (DCs)” refers to any member of a diverse population of morphologically similar cell types found in lymphoid or non-lymphoid tissues. DCs are a class of professional antigen presenting cells, and have a high capacity for sensitizing HLA-restricted T cells. DCs include, for example, plasmacytoid dendritic cells, myeloid dendritic cells (including immature and mature dendritic cells), Langerhans cells, interdigitating cells, follicular dendritic cells. Dendritic cells may be recognized by function, or by phenotype, particularly by cell surface phenotype. These cells are characterized by their distinctive morphology having veil-like projections on the cell surface, intermediate to high levels of surface HLA-class II expression and ability to present antigen to T cells, particularly to naive T cells (See Steinman R, et al., Ann. Rev.

Immunol. 1991; 9:271-196.). Typically, cell surface phenotype of DCs include CDla+, CD4+, CD86+, or HLA-DR. The term DCs encompasses both immature and mature DCs.

According to specific embodiments, the immune cells comprise granulocytes.

As used herein, the term “granulocytes” refer to polymorphonuclear leukocytes characterized by the presence of granules in their cytoplasm.

According to specific embodiments, the granulocytes comprise neutrophils.

According to specific embodiments, the granulocytes comprise mast-cells.

According to specific embodiments the immune cells comprise T cells.

As used herein, the term “T cells” refers to a differentiated lymphocyte with a CD3+, T cell receptor (TCR)+ having either CD4+ or CD8+ phenotype. The T cell may be either an effector or a regulatory T cell.

As used herein, the term “effector T cells” refers to a T cell that activates or directs other immune cells e.g. by producing cytokines or has a cytotoxic activity e.g., CD4+, Th1/Th2, CD8+ cytotoxic T lymphocyte.

As used herein, the term “regulatory T cell” or “Treg” refers to a T cell that negatively regulates the activation of other T cells, including effector T cells, as well as innate immune system cells.

Treg cells are characterized by sustained suppression of effector T cell responses. According to a specific embodiment, the Treg is a CD4+CD25+Foxp3+ T cell.

According to specific embodiments, the T cells are CD4+ T cells.

According to other specific embodiments, the T cells are CD8+ T cells.

According to specific embodiments, the T cells are memory T cells. Non-limiting examples of memory T cells include effector memory CD4+ T cells with a CD3+/CD4+/CD45RA−/CCR7-phenotype, central memory CD4+ T cells with a CD3+/CD4+/CD45RA−/CCR7+ phenotype, effector memory CD8+ T cells with a CD3+/CD8+CD45RA−/CCR7-phenotype and central memory CD8+ T cells with a CD3+/CD8+CD45RA−/CCR7+ phenotype.

According to specific embodiments, the T cells comprise engineered T cells transduced with a nucleic acid sequence encoding an expression product of interest.

According to specific embodiments, the expression product of interest is a T cell receptor (TCR) or a chimeric antigen receptor (CAR).

As used herein the phrase “transduced with a nucleic acid sequence encoding a TCR” or “transducing with a nucleic acid sequence encoding a TCR” refers to cloning of variable α- and β-chains from T cells with specificity against a desired antigen presented in the context of MHC. Methods of transducing with a TCR are known in the art and are disclosed e.g. in Nicholson et al. Adv Hematol. 2012; 2012:404081; Wang and Rivìere Cancer Gene Ther. 2015 March; 22(2):85-94); and Lamers et al, Cancer Gene Therapy (2002) 9, 613-623.

As used herein, the phrase “transduced with a nucleic acid sequence encoding a CAR” or “transducing with a nucleic acid sequence encoding a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing with a CAR are known in the art and are disclosed e.g. in Davila et al. Oncoimmunology. 2012 Dec. 1; 1(9):1577-1583; Wang and Rivière Cancer Gene Ther. 2015 March; 22(2):85-94); Maus et al. Blood. 2014 Apr. 24; 123(17):2625-35; Porter DL The New England journal of medicine. 2011, 365(8):725-733; Jackson H J, Nat Rev Clin Oncol. 2016; 13(6):370-383; and Globerson-Levin et al. Mol Ther. 2014; 22(5):1029-1038.

According to specific embodiments, the immune cells comprise B cells.

As used herein the term “B cells” refers to a lymphocyte with a B cell receptor (BCR)+, CD19+ and or B220+ phenotype. B cells are characterized by their ability to bind a specific antigen and elicit a humoral response.

According to specific embodiments, the immune cells comprise NK cells.

As used herein the term “NK cells” refers to differentiated lymphocytes with a CD16+CD56+ and/or CD57+ TCR-phenotype. NK are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

According to specific embodiments, the immune cells comprise NKT cells.

As used herein the term “NKT cells” refers to a specialized population of T cells that express a semi-invariant αβ T-cell receptor, but also express a variety of molecular markers that are typically associated with NK cells, such as NK1.1. NKT cells include NK1.1+ and NK1.1-, as well as CD4+, CD4-, CD8+ and CD8-cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance.

According to specific embodiments, the immune cells are obtained from a healthy subject.

According to specific embodiments, the immune cells are obtained from a subject suffering from a pathology (e.g. cancer).

According to specific embodiments, modulating is in the presence of cells expressing a ligand or a receptor of said type I membrane protein or said type II membrane protein or exogenous ligand or a receptor of said type I membrane protein or said type II membrane protein (e.g. PD-L1).

According to specific embodiments, the exogenous ligand or receptor is soluble.

According to other specific embodiments, the exogenous ligand or receptor is immobilized to a solid support.

According to specific embodiments, the cells expressing the ligand or receptor comprise pathologic (diseased) cells, e.g. cancer cells.

According to specific embodiments, the modulating is in the presence of a stimulatory agent capable of at least transmitting a primary activating signal [e.g. ligation of the T-Cell Receptor (TCR) with the Major Histocompatibility Complex (MHC)/peptide complex on the Antigen Presenting Cell (APC)] resulting in cellular proliferation, maturation, cytokine production, phagocytosis and/or induction of regulatory or effector functions of the immune cell. According to specific embodiments, the stimulator agent can also transmit a secondary co-stimulatory signal.

Methods of determining the amount of the stimulatory agent and the ratio between the stimulatory agent and the immune cells are well within the capabilities of the skilled in the art and thus are not specified herein.

The stimulatory agent can activate the immune cells in an antigen-dependent or -independent (i.e. polyclonal) manner.

According to specific embodiments, stimulatory agent comprises an antigen non-specific stimulator.

Non-specific stimulators are known to the skilled in the art. Thus, as a non-limiting example, when the immune cells comprise T cells, antigen non-specific stimulator can be an agent capable of binding to a T cell surface structure and induce the polyclonal stimulation of the T cell, such as but not limited to anti-CD3 antibody in combination with a co-stimulatory protein such as anti-CD28 antibody. Other non-limiting examples include anti-CD2, anti-CD137, anti-CD134, Notch-ligands, e.g. Delta-like 1/4, Jagged1/2 either alone or in various combinations with anti-CD3. Other agents that can induce polyclonal stimulation of T cells include, but not limited to mitogens, PHA, PMA-ionomycin, CEB and CytoStim (Miltenyi Biotech). According to specific embodiments, the antigen non-specific stimulator comprises anti-CD3 and anti-CD28 antibodies. According to specific embodiments, the T cell stimulator comprises anti-CD3 and anti-CD28 coated beads, such as the CD3CD28 MACSiBeads obtained from Miltenyi Biotec.

According to specific embodiments, the stimulatory agent comprises an antigen-specific stimulator.

Non-limiting examples of antigen specific T cell stimulators include an antigen-loaded antigen presenting cell [APC, e.g. dendritic cell] and peptide loaded recombinant MHC. Thus, for example, a T cells stimulator can be a dendritic cell preloaded with a desired antigen (e.g. a tumor antigen) or transfected with mRNA coding for the desired antigen.

According to specific embodiments, the antigen is a cancer antigen.

As used herein, the term “cancer antigen” refers to an antigen overexpressed or solely expressed by a cancerous cell as compared to a non-cancerous cell. A cancer antigen may be a known cancer antigen or a new specific antigen that develops in a cancer cell (i.e. neoantigens).

Non-limiting examples for known cancer antigens include MAGE-AI, MAGE-A2, MAGE-A3, MAGE-A4, MAGE-AS, MAGE-A6, MAGE-A7, MAGE-AS, MAGE-A9, MAGE-AIO, MAGE-All, MAGE-A12, GAGE-I, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-Cl/CT7, MAGE-C2, NY-ESO-1, LAGE-1, SSX-1, SSX-2 (HOM-MEL-40), SSX-3, SSX-4, SSX-5, SCP-1 and XAGE, melanocyte differentiation antigens, p53, ras, CEA, MUCI, PMSA, PSA, tyrosinase, Melan-A, MART-I, gplOO, gp75, alphaactinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, plSOerbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68KP1, CO-029, FGF-5, 0250, Ga733 (EpCAM), HTgp-175, M344, MA-50, MG7-Ag, MOV18, NB170K, NYCO-I, RCASI, SDCCAG16, TA-90 (Mac-2 binding protein\cyclophilin C-associated protein), TAAL6, TAG72, TLP, TPS, tyrosinase related proteins, TRP-1, or TRP-2.

Other tumor antigens that may be expressed are well-known in the art (see for example WO00/20581; Cancer Vaccines and Immunotherapy (2000) Eds Stern, Beverley and Carroll, Cambridge University Press, Cambridge). The sequences of these tumor antigens are readily available from public databases but are also found in WO 1992/020356 A1, WO 1994/005304 A1, WO 1994/023031 A1, WO 1995/020974 A1, WO 1995/023874 A1 & WO 1996/026214 A1.

Alternatively, or additionally, a tumor antigen may be identified using cancer cells obtained from the subject by e.g. biopsy.

Thus, according to specific embodiments, the stimulatory agent comprises a cancer cell.

According to specific embodiments, the modulating is in the presence of an anti-cancer agent.

According to specific embodiments, the immune cells are purified following the modulation.

Thus, the present invention also contemplates isolated immune cells obtainable according to the methods of the present invention.

According to specific embodiments, the immune cells used and/or obtained according to the present invention can be freshly isolated, stored e.g., cryopreserved (i.e. frozen) at e.g. liquid nitrogen temperature at any stage for long periods of time (e.g., months, years) for future use; and cell lines.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the cells obtained according to the present invention can be stored in a cell bank or a depository or storage facility.

Consequently, the present teachings further suggest the use of the isolated immune cells and the methods of the present invention as, but not limited to, a source for adoptive immune cells therapies for diseases that can benefit from modulating immune cells, for example from activating immune cells e.g. a hyper-proliferative disease; a disease associated with immune suppression and infections.

Thus, according to specific embodiments, method of the present invention comprises adoptively transferring the immune cells following said activating to a subject in need thereof.

According to specific embodiments, there is provided the immune cells obtainable according to the methods of the present invention for use in adoptive cell therapy.

The cells used according to specific embodiments of the present invention may be autologous or non-autologous; they can be syngeneic or non-syngeneic: allogeneic or xenogeneic to the subject; each possibility represents a separate embodiment of the present invention.

The present teachings also contemplate the use of the compositions of the present invention (e.g. the heterodimer, a nucleic acid construct or system encoding same or a host cell expressing same) in methods of treating a disease that can benefit from treatment with the heterodimer.

Thus, according to an aspect of the present invention, there is provided a method of treating a disease that can benefit from treatment with the heterodimer, the method comprising administering to a subject in need thereof the heterodimer, a nucleic acid construct or system encoding same or a host cell comprising same, thereby treating the disease in the subject.

According to an additional or an alternative aspect of the present invention, there is provided the heterodimer, a nucleic acid construct or system encoding same or a cell comprising same for use in treating a disease that can benefit from treatment with said heterodimer.

According to an additional or an alternative aspect of the present invention, there is provided a method of treating a disease that can benefit from modulating immune cells, the method comprising administering to a subject in need thereof the heterodimer, a nucleic acid construct or system encoding same or a host cell comprising same, thereby treating the disease in the subject.

According to an additional or an alternative aspect of the present invention, there is provided the heterodimer, a nucleic acid construct or system encoding same or a host cell comprising same for use in treating a disease that can benefit from modulating immune cells.

The term “treating” or “treatment” refers to inhibiting, preventing or arresting the development of a pathology (disease, disorder or medical condition) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, e.g., human beings at any age and of any gender. According to specific embodiments, the term “subject” refers to a subject who suffers from the pathology (disease, disorder or medical condition). According to specific embodiments, this term encompasses individuals who are at risk to develop the pathology.

According to specific embodiments, the subject is afflicted with a disease associated with cells expressing a ligand or a receptor of the type I membrane protein or the type II membrane protein.

According to specific embodiments, the subject is afflicted with a disease associated with cells expressing a ligand or a receptor of the type II membrane protein (e.g. 4-1BB, CD40).

According to specific embodiments, diseased cells of the subject express a ligand or a receptor of the type I membrane protein or the type II membrane protein.

According to specific embodiments, diseased cells of the subject express a ligand or a receptor of the type I membrane protein (e.g. PDL1, sialic acid, CD155).

According to specific embodiments, diseased cells of the subject express a ligand or a receptor of the type II membrane protein.

Non-limiting examples of diseases that can be treated according to specific embodiments of the present invention include diseases that can benefit from induction of angiogenesis (e.g. when the type I membrane protein is VEGFA and the type II membrane protein is TWEAK or APRIL), diseases that can benefit from inhibition of angiogenesis (e.g. when the type I membrane protein is ENG and the type II membrane protein is FasL, TRAIL or VEGI), for induction of bone formation (e.g. when the type I membrane protein is BMP2 and the type II membrane protein is TWEAK or APRIL), for inhibition of bone formation (e.g. when the type I membrane protein is BMP3 and the type II membrane protein is RNAKL, FasL, TRAIL, VEGI), for liver regeneration (e.g. when the type I membrane protein is GFER and the type II membrane protein is TWEAK or APRIL), and diseases that can benefit from modulating immune cells (e.g. when at least one of the type I membrane protein and the type II membrane protein is an immune modulator).

As used herein the phrase “a disease that can benefit from modulating immune cells” refers to diseases in which the subject's immune response activity may be sufficient to at least ameliorate symptoms of the disease or delay onset of symptoms, however for any reason the activity of the subject's immune response in doing so is less than optimal.

According to specific embodiments, the disease can benefit from activating immune cells.

Non-limiting examples of diseases that can benefit from activating immune cells include hyper-proliferative diseases, diseases associated with immune suppression, immunosuppression caused by medication (e.g. mTOR inhibitors, calcineurin inhibitor, steroids) and infections.

According to specific embodiments, the disease comprises a hyper-proliferative disease.

According to specific embodiments, the hyper-proliferative disease comprises sclerosis, fibrosis, Idiopathic pulmonary fibrosis, psoriasis, systemic sclerosis/scleroderma, primary biliary cholangitis, primary sclerosing cholangitis, liver fibrosis, prevention of radiation-induced pulmonary fibrosis, myelofibrosis or retroperitoneal fibrosis.

According to other specific embodiments, the hyper-proliferative disease comprises cancer.

Thus, according to another aspect of the present invention, there is provided a method of treating cancer comprising administering the PD1-4-1BBL fusion protein, the isolated polypeptide comprising the PD1 amino acid sequence and/or the isolated polypeptide comprising the 4-1BBL amino acid sequence disclosed herein to a subject in need thereof.

As used herein, the term cancer encompasses both malignant and pre-malignant cancers.

With regard to pre-malignant or benign forms of cancer, optionally the compositions and methods thereof may be applied for halting the progression of the pre-malignant cancer to a malignant form.

Cancers which can be treated by the methods of some embodiments of the invention can be any solid or non-solid cancer and/or cancer metastasis.

According to specific embodiments, the cancer comprises malignant cancer.

Cancers which can be treated by the methods of some embodiments of the invention can be any solid or non-solid cancer and/or cancer metastasis. Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; Burkitt lymphoma, Diffused large B cell lymphoma (DLBCL), high grade lymphoblastic NHL; high-grade small non-cleaved cell NHL; bulky disease NHL; mantle cell lymphoma; AIDS-related lymphoma; and Waldenstrom's Macroglobulinemia); T cell lymphoma, Hodgkin lymphoma, chronic lymphocytic leukemia (CLL); acute lymphoblastic leukemia (ALL); Acute myeloid leukemia (AML), Acute promyelocytic leukemia (APL), Hairy cell leukemia; chronic myeloblastic leukemia (CML); and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention include metastatic cancers.

According to specific embodiments, the cancer comprises pre-malignant cancer.

Pre-malignant cancers (or pre-cancers) are well characterized and known in the art (refer, for example, to Berman J J. and Henson D E., 2003. Classifying the precancers: a metadata approach. BMC Med Inform Decis Mak. 3:8). Classes of pre-malignant cancers amenable to treatment via the method of the invention include acquired small or microscopic pre-malignant cancers, acquired large lesions with nuclear atypia, precursor lesions occurring with inherited hyperplastic syndromes that progress to cancer, and acquired diffuse hyperplasias and diffuse metaplasias. Examples of small or microscopic pre-malignant cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to specific embodiments, the cancer is Acute Myeloid Leukemia, Anal Cancer, Basal Cell Carcinoma, B-Cell Non-Hodgkin Lymphoma, Bile Duct Cancer, Bladder Cancer, Breast Cancer, Cervical Cancer, Chronic Lymphocytic Leukemia (CLL), Chronic Myelocytic Leukemia (CML), Colorectal Cancer, Cutaneous T-Cell Lymphoma, Diffuse Large B-Cell Lymphoma, Endometrial Cancer, Esophageal Cancer, Fallopian Tube Cancer, Follicular Lymphoma, Gastric Cancer, Gastroesophageal (GE) Junction Carcinomas, Germ Cell Tumors, Germinomatous (Seminomatous), Germ Cell Tumors, Glioblastoma Multiforme (GBM), Gliosarcoma, Head And Neck Cancer, Hepatocellular Carcinoma, Hodgkin Lymphoma, Hypopharyngeal Cancer, Laryngeal Cancer, Leiomyosarcoma, Mantle Cell Lymphoma, Melanoma, Merkel Cell Carcinoma, Multiple Myeloma, Neuroendocrine Tumors, Non-Hodgkin Lymphoma, Non-Small Cell Lung Cancer, Oral Cavity (Mouth) Cancer, Oropharyngeal Cancer, Osteosarcoma, Ovarian Cancer, Pancreatic Cancer, Peripheral Nerve Sheath Tumor (Neurofibrosarcoma), Peripheral T-Cell Lymphomas (PTCL), Peritoneal Cancer, Prostate Cancer, Renal Cell Carcinoma, Salivary Gland Cancer, Skin Cancer, Small-Cell Lung Cancer, Soft Tissue Sarcoma, Squamous Cell Carcinoma, Synovial Sarcoma, Testicular Cancer, Thymic Carcinoma, Thyroid Cancer, Ureter Cancer, Urethral Cancer, Uterine Cancer, Vaginal Cancer or Vulvar Cancer.

According to specific embodiments, the cancer is Acute myeloid leukemia, Bladder Cancer, Breast Cancer, chronic lymphocytic leukemia, Chronic myelogenous leukemia, Colorectal cancer, Diffuse large B-cell lymphoma, Epithelial Ovarian Cancer, Epithelial Tumor, Fallopian Tube Cancer, Follicular Lymphoma, Glioblastoma multiform, Hepatocellular carcinoma, Head and Neck Cancer, Leukemia, Lymphoma, Mantle Cell Lymphoma, Melanoma, Mesothelioma, Multiple Myeloma, Nasopharyngeal Cancer, Non Hodgkin lymphoma, Non-small-cell lung carcinoma, Ovarian Cancer, Prostate Cancer or Renal cell carcinoma.

According to specific embodiments, the cancer is selected from the group consisting of lymphoma, leukemia and carcinoma.

According to specific embodiments, the cancer is selected from the group consisting of lymphoma, leukemia, colon cancer, pancreatic cancer, ovarian cancer, lung cancer and squamous cell carcinoma.

According to specific embodiments, the cancer is colon carcinoma.

According to specific embodiments, the cancer is ovarian carcinoma.

According to specific embodiments, the cancer is lung carcinoma.

According to specific embodiments, the cancer is head and neck carcinoma.

According to specific embodiments, the cancer is leukemia.

According to specific embodiments, the leukemia is selected from the group consisting of acute nonlymphocytic leukemia, chronic lymphocytic leukemia, acute granulocytic leukemia, chronic granulocytic leukemia, acute promyelocytic leukemia, adult T-cellleukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, ( )ross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

According to specific embodiments, the leukemia is promyelocytic leukemia, acute myeloid leukemia or chronic myelogenous leukemia.

According to specific embodiments, the cancer is lymphoma.

According to specific embodiments, the lymphoma is B cell lymphoma

According to specific embodiments, the lymphoma is T cell lymphoma.

According to other specific embodiments, the lymphoma is Hodgkins lymphoma.

According to specific embodiments, the lymphoma is non-Hodgkins lymphoma.

According to specific embodiments, the non-Hodgkin's Lymphoma is a selected from the group consisting of aggressive NHL, transformed NHL, indolent NHL, relapsed NHL, refractory NHL, low grade non-Hodgkin's Lymphoma, follicular lymphoma, large cell lymphoma, B-cell lymphoma, T-cell lymphoma, Mantle cell lymphoma, Burkitt's lymphoma, NK cell lymphoma, diffuse large B-cell lymphoma, acute lymphoblastic lymphoma, and cutaneous T cell cancer, including mycosos fungoides/Sezry syndrome.

According to specific embodiments, the cancer is multiple myeloma.

According to at least some embodiments, the multiple myeloma is selected from the group consisting of multiple myeloma cancers which produce light chains of kappa-type and/or light chains of lambda-type; aggressive multiple myeloma, including primary plasma cell leukemia (PCL); benign plasma cell disorders such as MGUS (monoclonal gammopathy of undetermined significance), Waldenstrom's macroglobulinemia (WM, also known as lymphoplasmacytic lymphoma) which may proceed to multiple myeloma; smoldering multiple myeloma (SMM), indolent multiple myeloma, premalignant forms of multiple myeloma which may also proceed to multiple myeloma; primary amyloidosis.

According to specific embodiments, the cancer is defined by the presence of tumors that have tumor-infiltrating lymphocytes (TILs) in the tumor micro-environment and/or tumors with a relatively high expression of ligand or receptor of the type I or type Ii membrane protein (e.g. PDL1 or CD47) in the tumor micro-environment.

According to specific embodiments, the disease comprises a disease associated with immune suppression or immunosuppression caused by medication (e.g. mTOR inhibitors, calcineurin inhibitor, steroids).

According to specific embodiments, the disease comprises HIV, Measles, influenza, LCCM, RSV, Human Rhinoviruses, EBV, CMV or Parvo viruses.

According to specific embodiments, the disease comprises an infection.

As used herein, the term “infection” or “infectious disease” refers to a disease induced by a pathogen. Specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, human immunodeficiency virus (HTV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the disease can benefit from inhibiting immune cells.

According to specific embodiments, the disease is an autoimmune disease. Such autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov DG. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza KM. et al., J Reprod Immunol 1998 February; 37 (2):87), autoimmune anti-sperm infertility (Diekman AB. et al., Am J Reprod Immunol. 2000 March; 43 (3):134), autoimmune prostatitis (Alexander RB. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau YE. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg CP. et al., Eur J Gastroenterol Hepatol. 1999 June; 11 (6):595) and autoimmune hepatitis (Manns MP. J Hepatol 2000 August; 33 (2):326).

Examples of autoimmune neurological diseases include, but are not limited to, multiple sclerosis (Cross AH. et al., J Neuroimmunol 2001 Jan. 1; 112 (1-2):1), Alzheimer's disease (Oron L. et al., J Neural Transm Suppl. 1997; 49:77), myasthenia gravis (Infante AJ. And Kraig E, Int Rev Immunol 1999; 18 (1-2):83; Oshima M. et al., Eur J Immunol 1990 December; 20 (12):2563), neuropathies, motor neuropathies (Kornberg AJ. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra HS. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine JC. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly CJ. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo TJ. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan OT. et al., Immunol Rev 1999 June; 169:107).

According to specific embodiments, the disease is graft rejection disease. Examples of diseases associated with transplantation of a graft include, but are not limited to, graft rejection, chronic graft rejection, subacute graft rejection, hyperacute graft rejection, acute graft rejection and graft versus host disease.

According to specific embodiments, the disease is an allergic disease, Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

According to specific embodiments, the compositions disclosed herein (e.g. heterodimer nucleic acid construct or system encoding same and/or host-cell expressing same) can be administered to a subject in combination with other established or experimental therapeutic regimen to treat the disease including, but not limited to analgesics, chemotherapeutic agents, radiotherapeutic agents, cytotoxic therapies (conditioning), hormonal therapy, antibodies and other treatment regimens (e.g., surgery) which are well known in the art.

According to specific embodiments, the therapeutic agent administered in combination with the composition of some embodiments of the invention comprises an antibody.

According to specific embodiments, the compositions disclosed herein (e.g. heterodimer, nucleic acid construct or system encoding same and/or host-cell expressing same) can be administered to a subject in combination with adoptive cell transplantation such as, but not limited to transplantation of bone marrow cells, hematopoietic stem cells, PBMCs, cord blood stem cells and/or induced pluripotent stem cells.

According to specific embodiments, the therapeutic agent administered in combination with the composition of some embodiments of the invention comprises an anti-cancer agent.

Anti-cancer agent that can be use with specific embodiments of the invention include, but are not limited to the anti-cancer drugs Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

According to specific embodiments, the anti-cancer agent comprises an antibody.

According to specific embodiments, the antibody is selected from the group consisting rituximab, cetuximab, trastuzumab, edrecolomab, alemtuzumab, gemtuzumab, ibritumomab, panitumumab Belimumab, Bevacizumab, Bivatuzumab mertansine, Blinatumomab, Blontuvetmab, Brentuximab vedotin, Catumaxomab, Cixutumumab, Daclizumab, Adalimumab, Bezlotoxumab, Certolizumab pegol, Citatuzumab bogatox, Daratumumab, Dinutuximab, Elotuzumab, Ertumaxomab, Etaracizumab, Gemtuzumab ozogamicin, Girentuximab, Necitumumab, Obinutuzumab, Ofatumumab, Pertuzumab, Ramucirumab, Siltuximab, Tositumomab, Nivolumab, Pembrolizumab, Durvalumab, Atezolizumab, Avelumab, Trastuzumab and ipilimumab.

According to specific embodiments, the antibody is selected from the group consisting of rituximab and cetuximab.

According to specific embodiments, the therapeutic agent or the anti-cancer agent comprises an IMiD (e.g. Thalidomide, Lenalidomie, Pomalidomide).

According to specific embodiments, the IMiD is selected from the group consisting of Thalidomide, Lenalidomie and Pomalidomide.

According to specific embodiments, the therapeutic agent administered in combination with the composition of some embodiments of the invention comprises an anti-infection agent (e.g. antibiotics and anti-viral agents).

According to specific embodiments, the therapeutic agent administered in combination with the composition of some embodiments of the invention comprises an immune suppressor agent (e.g. GCSF and other bone marrow stimulators, steroids).

According to specific embodiments the combination therapy has an additive effect.

According to specific embodiments, the combination therapy has a synergistic effect.

According to another aspect of the present invention there is provided an article of manufacture comprising a packaging material packaging a therapeutic agent for treating a disease; and the heterodimer, a nucleic acid construct or system encoding same or a host cell comprising same.

According to specific embodiments, the article of manufacture is identified for the treatment of a disease that can benefit from treatment with the heterodimer, e.g. a disease that can benefit from modulating immune cells.

According to specific embodiments, the therapeutic agent for treating said disease; and the heterodimer, the nucleic acid construct or system encoding same or the host cell expressing same are packaged in separate containers.

According to specific embodiments, the therapeutic agent for treating said disease; and the heterodimer, the nucleic acid construct or system encoding same or the host cell expressing same are packaged in a co-formulation.

According to specific embodiments, the heterodimer is attached to or comprises a heterologous therapeutic moiety. The therapeutic moiety may be any molecule, including small molecule chemical compounds and polypeptides.

Non-limiting examples of therapeutic moieties which can be used with specific embodiments of the invention include a cytotoxic moiety, a toxic moiety, a cytokine moiety, an immunomodultory moiety, a polypeptide, an antibody, a drug, a chemical and/or a radioisotope.

According to some embodiments of the invention, the therapeutic moiety is conjugated by translationally fusing the polynucleotide encoding the polypeptide of some embodiments of the invention with the nucleic acid sequence encoding the therapeutic moiety.

Additionally or alternatively, the therapeutic moiety can be chemically conjugated (coupled) to the heterodimer of some embodiments of the invention, using any conjugation method known to one skilled in the art. For example, a peptide can be conjugated to an agent of interest, using a 3-(2-pyridyldithio) propionic acid Nhydroxysuccinimide ester (also called N-succinimidyl 3-(2-pyridyldithio) propionate) (“SDPD”) (Sigma, Cat. No. P-3415; see e.g., Cumber et al. 1985, Methods of Enzymology 112: 207-224), a glutaraldehyde conjugation procedure (see e.g., G. T. Hermanson 1996, “Antibody Modification and Conjugation, in Bioconjugate Techniques, Academic Press, San Diego) or a carbodiimide conjugation procedure [see e.g., J. March, Advanced Organic Chemistry: Reaction's, Mechanism, and Structure, pp. 349-50 & 372-74 (3d ed.), 1985; B. Neises et al. 1978, Angew Chem., Int. Ed. Engl. 17:522; A. Hassner et al. 1978, Tetrahedron Lett. 4475; E. P. Boden et al. 1986, J. Org. Chem. 50:2394 and L. J. Mathias 1979, Synthesis 561].

A therapeutic moiety can be attached, for example, to the heterodimer of some embodiments of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like.

As used herein, the terms “amino acid sequence”, “protein”, “peptide”, “polypeptide” and “proteinaceous moiety”, which are interchangeably used herein, encompass native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino | Xaa | X |
| acid as above | | |

TABLE 2

| Non-conventional | | Non-conventional | |
|---|---|---|---|
| amino acid | Code | amino acid | Code |
| ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl)glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino- | Nmaabu |
| | | α-methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl | Nmbc | 1,2,3,4-tetrahydroisoquinoline- | Tic |
| ethylamino)cyclopropane | | 3-carboxylic acid | |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention are preferably utilized in a linear form, although it will be appreciated that in cases where cyclicization does not severely interfere with peptide characteristics, cyclic forms of the peptide can also be utilized.

Since the present heterodimers are preferably utilized in therapeutics which require the heterodimer to be in soluble form, the peptides of some embodiments of the invention include one or more non-natural or natural polar amino acids, including but not limited to serine and threonine which are capable of increasing peptide solubility due to their hydroxyl-containing side chain.

The amino acids of the peptides of the present invention may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions, the substituting amino acid should have the same or a similar functional group in the side chain as the original amino acid.

Conservative substitution tables providing functionally similar amino acids are well known in the art. Guidance concerning which amino acid changes are likely to be phenotypically silent can also be found in Bowie et al., 1990, Science 247: 1306 1310. Such conservatively modified variants are in addition to and do not exclude polymorphic variants, interspecies homologs, and alleles. Typical conservative substitutions include but are not limited to: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Amino acids can be substituted based upon properties associated with side chains, for example, amino acids with polar side chains may be substituted, for example, Serine (S) and Threonine (T); amino acids based on the electrical charge of a side chains, for example, Arginine (R) and Histidine (H); and amino acids that have hydrophobic side chains, for example, Valine (V) and Leucine (L). As indicated, changes are typically of a minor nature, such as conservative amino acid substitutions that do not significantly affect the folding or activity of the protein.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH [(—CH₂)₅—COOH]—CO— for aspartic acid. Those non-conservative substitutions which fall under the scope of the present invention are those which still constitute a peptide having anti-bacterial properties.

The N and C termini of the peptides of the present invention may be protected by function groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

According to specific embodiments, one or more of the amino acids may be modified by the addition of a functional group, for example (conceptually views as “chemically modified”). For example, the side amino acid residues appearing in the native sequence may optionally be modified, although as described below alternatively other parts of the protein may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible. Modifications to the peptide or protein can be introduced by gene synthesis, site-directed (e.g., PCR based) or random mutagenesis (e.g., EMS) by exonuclease deletion, by chemical modification, or by fusion of polynucleotide sequences encoding a heterologous domain or binding protein, for example.

As used herein the term “chemical modification”, when referring to a peptide, refers to a peptide where at least one of its amino acid residues is modified either by natural processes, such as processing or other post-translational modifications, or by chemical modification techniques which are well known in the art. Non-limiting exemplary types of modification include carboxymethylation, acetylation, acylation, phosphorylation, glycosylation, amidation, ADP-ribosylation, fatty acylation, addition of farnesyl group, an isofarnesyl group, a carbohydrate group, a fatty acid group, a linker for conjugation, functionalization, GPI anchor formation, covalent attachment of a lipid or lipid derivative, methylation, myristylation, pegylation, prenylation, phosphorylation, ubiquitination, or any similar process and known protecting/blocking groups. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

According to specific embodiments, the modifications include the addition of a cycloalkane moiety to the peptide, as described in PCT Application No. WO 2006/050262, hereby incorporated by reference as if fully set forth herein. These moieties are designed for use with biomolecules and may optionally be used to impart various properties to proteins.

Furthermore, optionally any point on the peptide may be modified. For example, pegylation of a glycosylation moiety on a protein may optionally be performed, as described in PCT Application No. WO 2006/050247, hereby incorporated by reference as if fully set forth herein. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

By “PEGylated protein” is meant a protein, or a fragment thereof having biological activity, having a polyethylene glycol (PEG) moiety covalently bound to an amino acid residue of the protein.

By “polyethylene glycol” or “PEG” is meant a polyalkylene glycol compound or a derivative thereof, with or without coupling agents or derivatization with coupling or activating moieties (e.g., with thiol, triflate, tresylate, azirdine, oxirane, or preferably with a maleimide moiety). Compounds such as maleimido monomethoxy PEG are exemplary or activated PEG compounds of the invention. Other polyalkylene glycol compounds, such as polypropylene glycol, may be used in the present invention. Other appropriate polyalkylene glycol compounds include, but are not limited to, charged or neutral polymers of the following types: dextran, colominic acids or other carbohydrate-based polymers, polymers of amino acids, and biotin derivatives.

According to specific embodiments, the peptide is modified to have an altered glycosylation pattern (i.e., altered from the original or native glycosylation pattern). As used herein, “altered” means having one or more carbohydrate moieties deleted, and/or having at least one glycosylation site added to the original protein.

Glycosylation of proteins is typically either N-linked or O-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to a peptide is conveniently accomplished by altering the amino acid sequence of the peptide such that it contains one or more of the above-described tripeptide sequences (for N-linked glycosylation sites). The alteration may also be made by the addition of, or substitution by, one or more serine or threonine residues in the sequence of the original peptide (for O-linked glycosylation sites). The peptide's amino acid sequence may also be altered by introducing changes at the DNA level.

Another means of increasing the number of carbohydrate moieties on peptides is by chemical or enzymatic coupling of glycosides to the amino acid residues of the peptide. Depending on the coupling mode used, the sugars may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described e.g. in WO 87/05330, and in Aplin and Wriston, CRC Crit. Rev. Biochem., 22: 259-306 (1981).

Removal of any carbohydrate moieties present on a peptide may be accomplished chemically, enzymatically or by introducing changes at the DNA level. Chemical deglycosylation requires exposure of the peptide to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259: 52 (1987); and Edge et al., Anal. Biochem., 118: 131 (1981). Enzymatic cleavage of carbohydrate moieties on peptides can be achieved by the use of a variety of endo- and exo-glycosidases as described by Thotakura et al., Meth. Enzymol., 138: 350 (1987).

According to specific embodiments, the peptide comprises a detectable tag. As used herein, in one embodiment the term “detectable tag” refers to any moiety that can be detected by a skilled practitioner using art known techniques. Detectable tags may be peptide sequences. Optionally the detectable tag may be removable by chemical agents or by enzymatic means, such as proteolysis. Detectable tags of some embodiments of the present invention can be used for purification of the peptide. For example the term “detectable tag” includes chitin binding protein (CBP)-tag, maltose binding protein (MBP)-tag, glutathione-S-transferase (GST)-tag, poly(His)-tag, FLAG tag, Epitope tags, such as, V5-tag, c-myc-tag, and HA-tag, and fluorescence tags such as green fluorescent protein (GFP), red fluorescent protein (RFP), yellow fluorescent protein (YFP), blue fluorescent protein (BFP), and cyan fluorescent protein (CFP); as well as derivatives of these tags, or any tag known in the art. The term “detectable tag” also includes the term “detectable marker”.

According to specific embodiment, the peptide comprises a detectable tag attached to its N-terminal (e.g. poly(His)-tag).

According to specific embodiment, the peptide comprises a detectable tag attached to its C-terminal (e.g. poly(His)-tag).

According to specific embodiments, the N-terminal of the peptide does not comprise a detectable tag (e.g. poly(His)-tag).

According to specific embodiments, the C-terminal of the peptide does not comprise a detectable tag (e.g. poly(His)-tag).

According to specific embodiments the peptide is fused to a cleavable moiety. Thus, for example, to facilitate recovery, the expressed coding sequence can be engineered to encode the peptide of some embodiments of the present invention and fused cleavable moiety. In one embodiment, the peptide is designed such that it is readily isolated by affinity chromatography; e.g., by immobilization on a column specific for the cleavable moiety. In one embodiment, a cleavage site is engineered between the peptide and the cleavable moiety and the peptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)]. According to specific embodiments, the peptide is an isolated peptide.

The peptides and heterodimers comprising same of some embodiments of the invention may be synthesized and purified by any techniques that are known to those skilled in the art of peptide synthesis, such as, but not limited to, solid phase and recombinant techniques.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

According to specific embodiments, the peptide is synthesized using in vitro expression systems. Such in vitro synthesis methods are well known in the art and the components of the system are commercially available.

According to specific embodiments, the peptides or the heterodimers comprising same are produced by recombinant DNA technology. A “recombinant” peptide, or protein refers to a peptide, or protein produced by recombinant DNA techniques; i.e., produced from cells transformed by an exogenous DNA construct encoding the desired peptide or protein.

Thus, according to another aspect of the present invention, there is provided a nucleic acid construct or system comprising at least one polynucleotide encoding the heterodimer, and a regulatory element for directing expression of said polynucleotide in a host cell.

According to specific embodiments, the polynucleotide is at least about 70%, 75%, 80%, 85%, 90%, 95%, 96%, 97%, 98% or 99% homologous to the nucleic acid sequence as set forth in SEQ ID NO: 80 and 82 or SEQ ID NO: 80 and 84, each possibility represents a separate embodiment of the present invention.

According to specific embodiments, the polynucleotide is at least about 70%, 75%, 80%, 85%, 90%, 95%, 96%, 97%, 98% or 99% homologous to the nucleic acid sequence as set forth in SEQ ID NO: 86 and 82, SEQ ID NO: 90 and 92 or SEQ ID NO: 86 and 84, each possibility represents a separate embodiment of the present invention.

According to specific embodiments, the polynucleotide comprises SEQ ID NO: 80 and 82 or SEQ ID NO: 80 and 84, each possibility represents a separate embodiment of the present invention.

According to specific embodiments, the polynucleotide comprises SEQ ID NO: 86 and 82, SEQ ID NO: 90 and 92 or SEQ ID NO: 86 and 84, each possibility represents a separate embodiment of the present invention.

According to specific embodiments, the polynucleotide is at least about 70%, 75%, 80%, 85%, 90%, 95%, 96%, 97%, 98% or 99% homologous to the nucleic acid sequence as set forth in SEQ ID NO: 147 and 82, SEQ ID NO: 143 and 139, SEQ ID NO: 145 and 141, SEQ ID NO: 86 and 139, SEQ ID NO: 86 and 141, SEQ ID NO: 147 and 139, SEQ ID NO: 149 and 139, SEQ ID NO: 80 and 139, SEQ ID NO: 155 and 139, SEQ ID NO: 80 and 151, SEQ ID NO: 155 and 151, SEQ ID NO: 80 and 153, SEQ ID NO: 157 and 82, SEQ ID NO: 155 and 153 or SEQ ID NO: 159 and 82, each possibility represents a separate embodiment of the present invention.

According to specific embodiments, the polynucleotide comprises in SEQ ID NO: 147 and 82, SEQ ID NO: 143 and 139, SEQ ID NO: 145 and 141, SEQ ID NO: 86 and 139, SEQ ID NO: 86 and 141, SEQ ID NO: 147 and 139, SEQ ID NO: 149 and 139, SEQ ID NO: 80 and 139, SEQ ID NO: 155 and 139, SEQ ID NO: 80 and 151, SEQ ID NO: 155 and 151, SEQ ID NO: 80 and 153, SEQ ID NO: 157 and 82, SEQ ID NO: 155 and 153 or SEQ ID NO: 159 and 82, each possibility represents a separate embodiment of the present invention.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to specific embodiments, any of the polynucleotides and nucleic acid sequences disclosed herein may comprise conservative nucleic acid substitutions. Conservatively modified polynucleotides refer to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified polynucleotides. According to specific embodiments, any polynucleotide and nucleic acid sequence described herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a polynucleotide which encodes a polypeptide is implicit in a described sequence with respect to the expression product.

To express an exogenous polypeptide in mammalian cells, a polynucleotide sequence encoding the polypeptide is preferably ligated into a nucleic acid construct suitable for mammalian cell expression. Such a nucleic acid construct includes a promoter sequence for directing transcription of the polynucleotide sequence in the cell in a constitutive or inducible manner.

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

Thus, according to specific embodiments, both monomers comprised in the heterodimer are expressed from a single construct.

According to other specific embodiments, each of the monomers comprised in the heterodimer is expressed from a different construct.

It will be appreciated that the individual elements comprised in the expression vector can be arranged in a variety of configurations. For example, enhancer elements, promoters and the like, and even the polynucleotide sequence(s) encoding the monomers or the heterodimer arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the monomers and heterodimers since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

As mentioned, other than containing the necessary elements for the transcription and translation of the inserted coding sequence, the expression construct of some embodiments of the invention can also include sequences engineered to enhance stability, production, purification, yield or toxicity of the expressed monomer or heterodimer. For example, the expression of a fusion protein or a cleavable fusion protein comprising the monomer or heterodimer of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the monomer or heterodimer of some embodiments of the present invention and the heterologous protein, the monomer or heterodimer can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

The present invention also contemplates cells comprising the composition described herein.

Thus, according to an aspect of the present invention, there is provided a host cell comprising the heterodimer or the nucleic acid construct or system.

As mentioned hereinabove, a variety of prokaryotic or eukaryotic cells can be used as host-expression systems to express the heterodimer of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al. (1990) Methods in Enzymol. 185:60-89).

Examples of eukaryotic cells which may be used along with the teachings of the invention include but are not limited to, mammalian cells, fungal cells, yeast cells, insect cells, algal cells or plant cells.

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hspl7.5-E or hspl7.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art can also be used by some embodiments of the invention.

According to specific embodiments the cell is a mammalian cell.

According to specific embodiment, the cell is a human cell.

According to a specific embodiment, the cell is a cell line.

According to another specific embodiment, the cell is a primary cell.

The cell may be derived from a suitable tissue including but not limited to blood, muscle, nerve, brain, heart, lung, liver, pancreas, spleen, thymus, esophagus, stomach, intestine, kidney, testis, ovary, hair, skin, bone, breast, uterus, bladder, spinal cord, or various kinds of body fluids. The cells may be derived from any developmental stage including embryo, fetal and adult stages, as well as developmental origin i.e., ectodermal, mesodermal, and endodermal origin.

Non limiting examples of mammalian cells include monkey kidney CV1 line transformed by SV40 (COS, e.g. COS-7, ATCC CRL 1651); human embryonic kidney line (HEK293 or HEK293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HeLa, ATCC CCL 2); NIH3T3, Jurkat, canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2), PER.C6, K562, and Chinese hamster ovary cells (CHO).

According to some embodiments of the invention, the mammalian cell is selected from the group consisting of a Chinese Hamster Ovary (CHO), HEK293, PER.C6, HT1080, NS0, Sp2/0, BHK, Namalwa, COS, HeLa and Vero cell.

According to some embodiments of the invention, the host cell comprises a Chinese Hamster Ovary (CHO), PER.C6 or a 293 (e.g. Expi293F) cell.

According to another aspect of the present invention, there is provided method of producing a heterodimer, the method comprising expressing in a host cell the nucleic acid construct or system.

According to specific embodiments, the producing comprises expressing in a mammalian cell and culturing at 32-37° C., 5-10% CO2 for 5-13 days.

Non-limiting examples of production conditions that can be used with specific embodiments of the invention are disclosed in the Examples section which follows.

Thus, for example an expression vector encoding the heterodimer, is expressed in mammalian cells such as Expi293F or ExpiCHO cells. The transduced cells are then cultured at 32-37° C. 5-10% CO2 in cell-specific culture medium according to the Expi293F or ExpiCHO cells manufacturer instructions (Thermo) and following at least 5 days in culture the proteins are collected from the supernatant and purified.

According to specific embodiments the culture is operated in a batch, split-batch, fed-batch, or perfusion mode.

According to specific embodiments, the culture is operated under fed-batch conditions.

According to specific embodiments, the culturing is effected at 36.5° C.

According to specific embodiments, the culturing it effected at 36. 5° C. with a temperature shift to 32° C. This temperature shift can be effected to slow down cells metabolism prior to reaching a stationary phase.

According to specific embodiments, the method comprising adding the dimerizing moiety to the expressed amino acid sequences, i.e. to the amino acid sequence of type I membrane protein and the amino acid sequence of type II membrane protein.

According to specific embodiments, the methods comprising isolating the heterodimer.

According to specific embodiments, recovery of the recombinant heterodimer is effected following an appropriate time in culture. According to specific embodiments, recovering the recombinant heterodimer refers to collecting the whole culture medium containing the heterodimer and need not imply additional steps of separation or purification. According to specific embodiments, heterodimers of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, mix mode chromatography, metal affinity chromatography, Lectins affinity chromatography chromatofocusing and differential solubilization.

According to specific embodiments, following production and purification, the therapeutic efficacy of the heterodimer can be assayed either in vivo or in vitro. Such methods are known in the art and include for example cell viability, survival of transgenic mice, and expression of activation markers.

The compositions (e.g. the heterodimer, nucleic acid construct or system encoding same and/or cells) of some embodiments of the invention can be administered to an organism per se, or in a pharmaceutical composition where it is mixed with suitable carriers or excipients.

Thus, the present invention, in some embodiments, features a pharmaceutical composition comprising a therapeutically effective amount of the composition disclosed herein.

Herein the term “active ingredient” refers to the composition (e.g. heterodimer, nucleic acid construct or system and/or cells described herein) accountable for the biological effect.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the present invention also may include a pharmaceutically acceptable anti-oxidants. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. A pharmaceutical composition according to at least some embodiments of the present invention also may include additives such as detergents and solubilizing agents (e.g., TWEEN 20 (polysorbate-20), TWEEN 80 (polysorbate-80)) and preservatives (e.g., Thimersol, benzyl alcohol) and bulking substances (e.g., lactose, mannitol).

Examples of suitable aqueous and nonaqueous carriers that may be employed in the pharmaceutical compositions according to at least some embodiments of the present invention include water, buffered saline of various buffer content (e.g., Tris-HCl, acetate, phosphate), pH and ionic strength, ethanol, polyols (such as glycerol, propylene glycol, polyethylene glycol, and the like), and suitable mixtures thereof, vegetable oils, such as olive oil, and injectable organic esters, such as ethyl oleate.

Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the present invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

Techniques for formulation and administration of drugs may be found in “Remington's Pharmaceutical Sciences,” Mack Publishing Co., Easton, Pa., latest edition, which is incorporated herein by reference.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

A composition of the present invention can be administered via one or more routes of administration using one or more of a variety of methods known in the art. As will be appreciated by the skilled artisan, the route and/or mode of administration will vary depending upon the desired results. Preferred routes of administration for therapeutic agents according to at least some embodiments of the present invention include intravascular delivery (e.g. injection or infusion), intravenous, intramuscular, intradermal, intraperitoneal, subcutaneous, spinal, oral, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intraperitoneal, intravenous (IV) and intradermal), transdermal (either passively or using iontophoresis or electroporation), transmucosal (e.g., sublingual administration, nasal, vaginal, rectal, or sublingual), administration or administration via an implant, or other parenteral routes of administration, for example by injection or infusion, or other delivery routes and/or forms of administration known in the art. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion or using bioerodible inserts, and can be formulated in dosage forms appropriate for each route of administration. In a specific embodiment, a protein, a therapeutic agent or a pharmaceutical composition according to at least some embodiments of the present invention can be administered intraperitoneally or intravenously.

According to specific embodiments, the compositions disclosed herein are administered in an aqueous solution, by parenteral injection. The formulation may also be in the form of a suspension or emulsion. In general, pharmaceutical compositions for parenteral injection are provided including effective amounts of the compositions described herein, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., water soluble antioxidants such as ascorbic acid, sodium metabisulfite, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, alpha-tocopherol; and metal chelating agents, such as citric acid, ethylenediamine tetraacetic acid (EDTA), sorbitol, tartaric acid, phosphoric acid), and preservatives (e.g., Thimersol, benzyl alcohol) and bulking substances (e.g., lactose, mannitol). Examples of non-aqueous solvents or vehicles are ethanol, propylene glycol, polyethylene glycol, vegetable oils, such as olive oil and corn oil, gelatin, and injectable organic esters such as ethyl oleate. The formulations may be freeze dried (lyophilized) or vacuum dried and redissolved/resuspended immediately before use. The formulation may be sterilized by, for example, filtration through a bacteria retaining filter, by incorporating sterilizing agents into the compositions, by irradiating the compositions, or by heating the compositions.

Various compositions (e.g., polypeptides) disclosed herein can be applied topically. Topical administration does not work well for most peptide formulations, although it can be effective especially if applied to the lungs, nasal, oral (sublingual, buccal), vaginal, or rectal mucosa.

Compositions of the present invention can be delivered to the lungs while inhaling and traverse across the lung epithelial lining to the blood stream when delivered either as an aerosol or spray dried particles having an aerodynamic diameter of less than about 5 microns. A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

According to specific embodiments, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans. Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

In certain embodiments, the composition (e.g. heterodimer, the nucleic acid construct or system or cells) is administered locally, for example by injection directly into a site to be treated. Typically, the injection causes an increased localized concentration of the composition which is greater than that which can be achieved by systemic administration. The heterodimer compositions can be combined with a matrix as described above to assist in creating an increased localized concentration of the polypeptide compositions by reducing the passive diffusion of the polypeptides out of the site to be treated.

Pharmaceutical compositions of the present invention may be administered with medical devices known in the art. For example, in an optional embodiment, a pharmaceutical composition according to at least some embodiments of the present invention can be administered with a needle hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of the active agents disclosed herein, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl Polymer ScL, 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

In certain embodiments, to ensure that the therapeutic compounds according to at least some embodiments of the present invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Selection Variants of Heterodimers-Proteins Containing Two or Three Proteins Linked by Fc

Structural analysis of heterodimers-proteins containing:
extra cellular domain (ECD) of PD1 and a single chain comprising 3 repeats of ECD of 4-1BBL (referred to herein as “sc3x4-1BBL”) linked by FC chains (referred to herein as “DSP305”, SEQ ID NOs: 79 and 81);
combinations of ECDs of PD1, SIRPα and sc3x4-1BBL linked by FC chains (referred to herein as “TSP111”, SEQ ID NOs: 85 and 81), comprising an N-terminal signal peptide (SEQ ID NO: 95) and “knob into hole” containing FC of hIgG4 (SEQ ID NOs: 110 and 111);
combinations of ECDs of PD1, SIRPα and sc3xCD40L linked by FC chains (referred to herein as “TSP112”, SEQ ID NOs: 81 and 146), comprising an N-terminal signal peptide (SEQ ID NO: 95) and “knob into hole” containing FC of hIgG4 (SEQ ID NOs: 110 and 111);
combinations of ECDs of LILRB2, SIRPα and sc3x4-1BBL linked by FC chains (referred to herein as “TSP215”, SEQ ID NOs: 138 and 85), comprising an N-terminal signal peptide (SEQ ID NO: 95) and “knob into hole” containing FC of hIgG4 (SEQ ID NOs: 110 and 111);
combinations of ECDs of LILRB2, SIRPα and sc3xCD40L linked by FC chains (referred to herein as “TSP217”, SEQ ID NOs: 138 and 146), comprising an N-terminal signal peptide (SEQ ID NO: 95) and “knob into hole” containing FC of hIgG4 (SEQ ID NOs: 110 and 111);
combinations of ECDs of SIGLEC10, SIRPα and sc3x4-1BBL linked by FC chains (referred to herein as “TSP401”, SEQ ID NOs: 150 and 79), comprising an N-terminal signal peptide (SEQ ID NO: 95) and “knob into hole” containing FC of hIgG4 (SEQ ID NOs: 110 and 111);
combinations of ECDs of TIGIT, PD1 and sc3x4-1BBL linked by FC chains (referred to herein as “TSP501”, SEQ ID NOs: 152 and 79), comprising an N-terminal signal peptide (SEQ ID NO: 95) and “knob into hole” containing FC of hIgG4 (SEQ ID NOs: 110 and 111);
combinations of ECDs of LILRB2, PD1 and 3xscCD40L linked by FC chains (referred to herein as “TSP222”, SEQ ID NOs: 138 and 154); comprising an N-terminal signal peptide (SEQ ID NO: 95) and “knob into hole” containing FC of hIgG4 (SEQ ID NOs: 110 and 111);
combinations of ECDs of SIGLEC10, PD1 and 3xscCD40L linked by FC chains (referred to herein as “TSP403”, SEQ ID NOs: 150 and 154), comprising an N-terminal signal peptide (SEQ ID NO: 95) and “knob into hole” containing FC of hIgG4 (SEQ ID NOs: 110 and 111);
combinations of ECDs of TIGIT, PD1 and 3xscCD40L linked by FC chains (referred to herein as “TSP503”, SEQ ID NOs: 152 and 154), comprising an N-terminal signal peptide (SEQ ID NO: 95) and “knob into hole” containing FC of hIgG4 (SEQ ID NOs: 110 and 111);
combinations of ECDs of PD1, TIGIT and 3xsc4-1BBL linked by FC chains (referred to herein as “TSP501V1”, SEQ ID NOs: 81 and 156) comprising an N-terminal signal peptide (SEQ ID NO: 95) and “knob into hole” containing FC of hIgG4 (SEQ ID NOs: 110 and 111); or
combinations of ECDs of PD1, TIGIT and 3xscCD40L linked by FC chains (referred to herein as “TSP503V1”, SEQ ID NOs: 81 and 158) comprising an N-terminal signal peptide (SEQ ID NO: 95) and “knob into hole” containing FC of hIgG4 (SEQ ID NOs: 110 and 111);
   was effected in order to optimize the following parameters:
Folding—proper folding to allow binding to targets, minimize potential di-sulfide scrambling;
Integrity—no exposed proteolytic sites;
High expression in mammalian expression system; and
Low immunogenicity.

Homology modeling was performed for each part based on a homologue X-ray structure. For PD1-PDB IDs: 3RRQ, 5GGR, 5GGS, 5JXE and 4ZQK were used as templates. For hIgG4-PDB IDs: 4C54, 4C55, 5W5M and 5W5N were used as templates. For 41BB-L-PDB IDs: 6CPR, 6A3V and 6CU0 were used as templates. For SIRPα-PDB ID's 2UV3, 2WNG, 4CMM, 6BIT, 2JJS and 2JJT were used as templates. Linker segments were modeled using loop modeling in CHARMM primarily in order to avoid structural violations and to enable a plausible estimation for a possible ‘spacer’ length. For CD40L-PDB IDs: 1ALY, 118R, 3LKJ and 3QD6 were used as templates. For LILRB2-PDB IDs: 2GW5, 4LLA, 2DYP and 6BCP were used as templates. For SIGLEC10-PDB IDs: 2N7A and 2N7B of the SIGLEC8 homologue were used as templates. For TIGIT-PDB IDs: 3QOH, 3RQ3, 3UCR, 3UDW and 5V52 were used as templates.

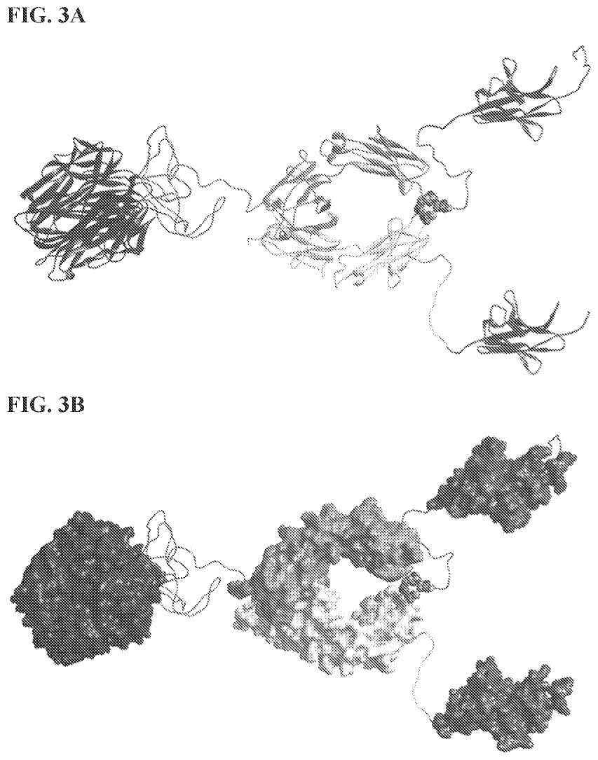

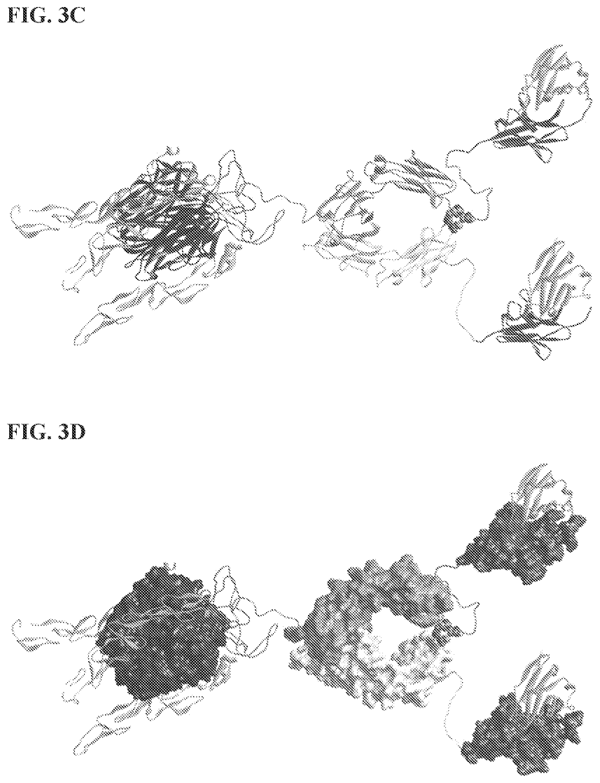

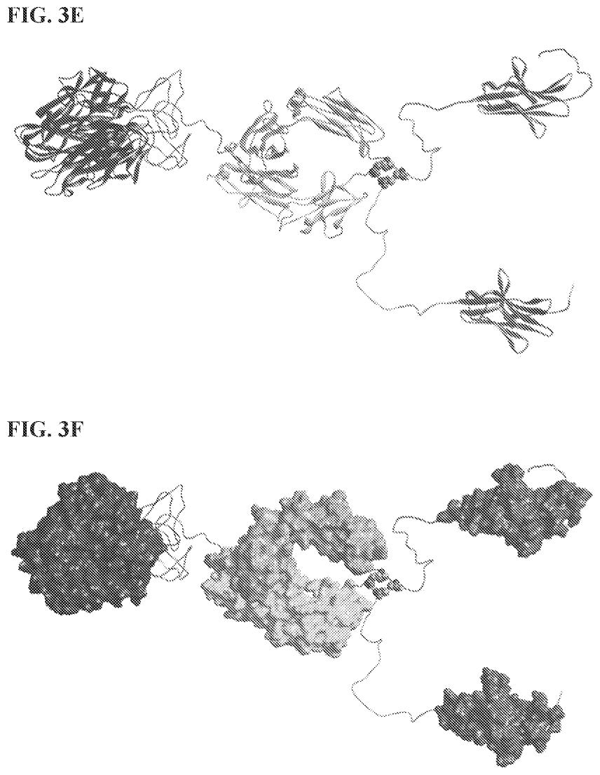

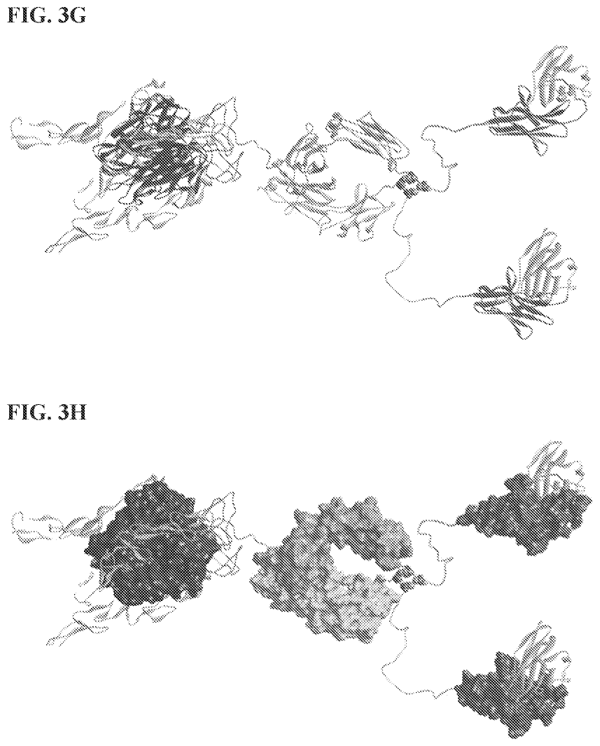

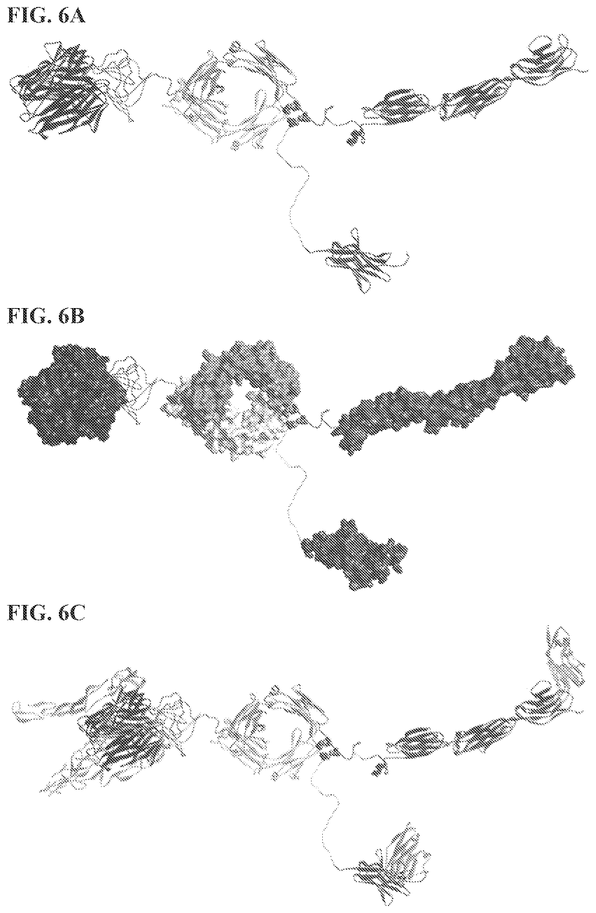

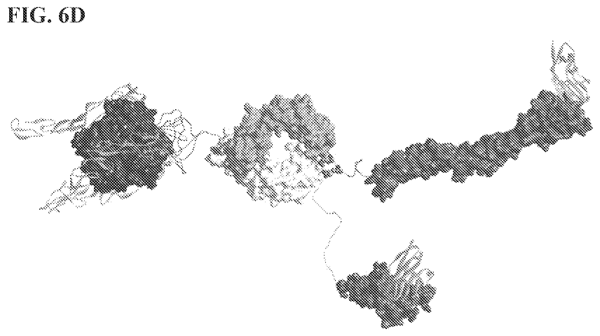

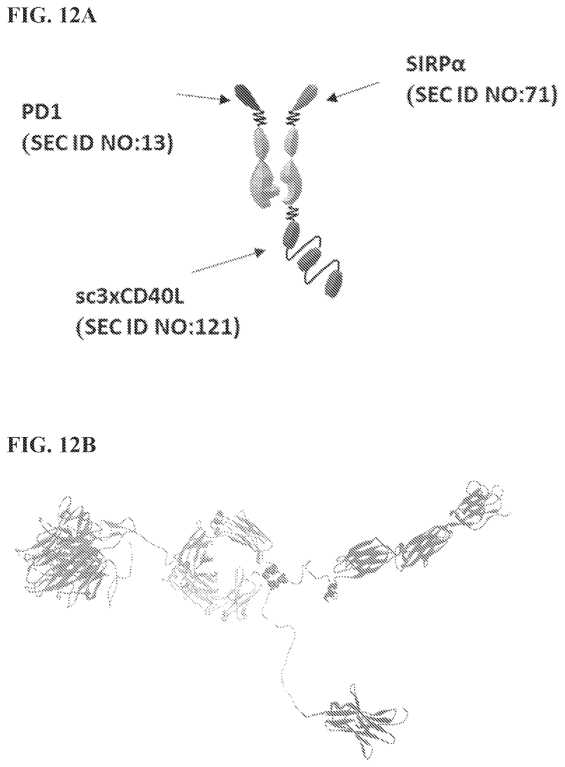

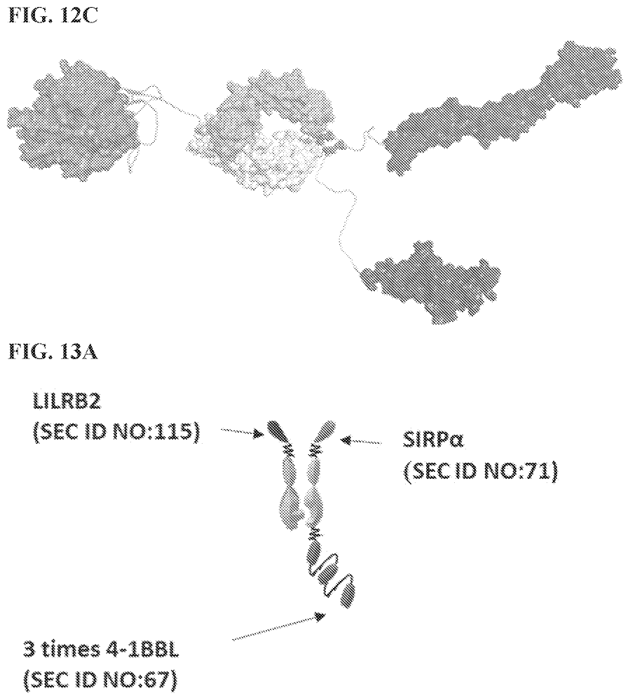

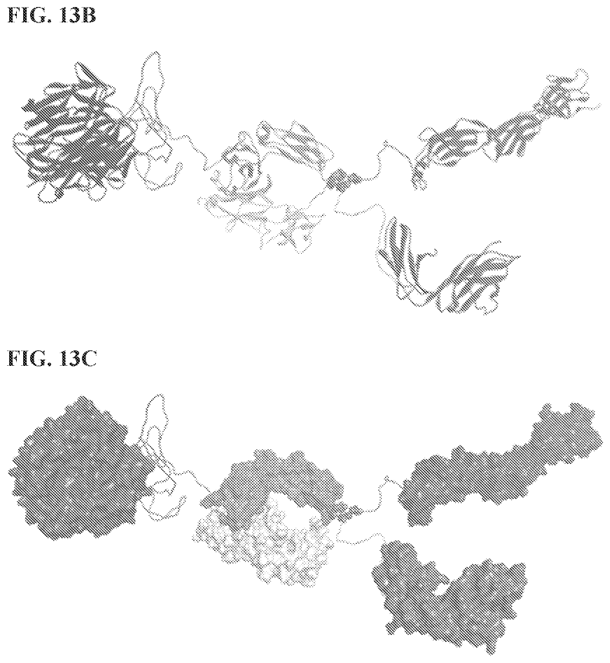

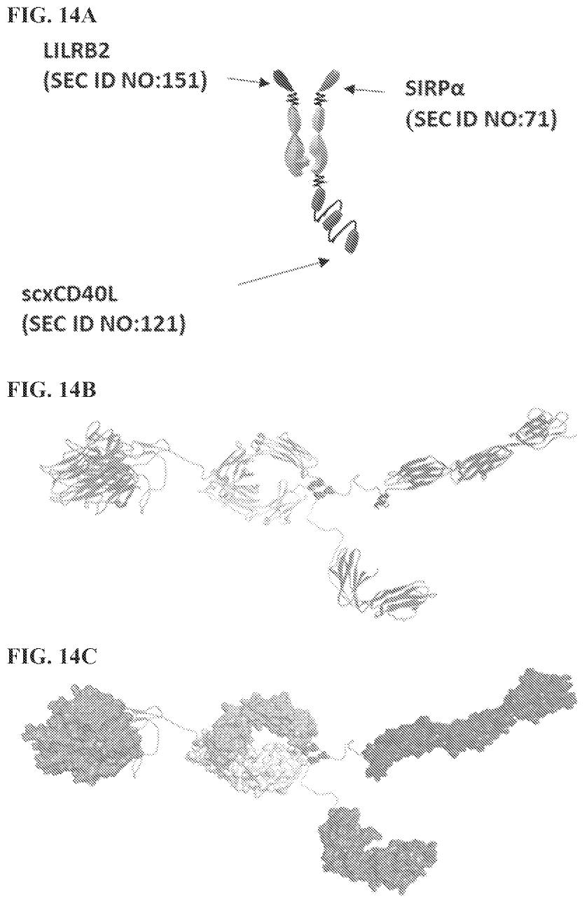

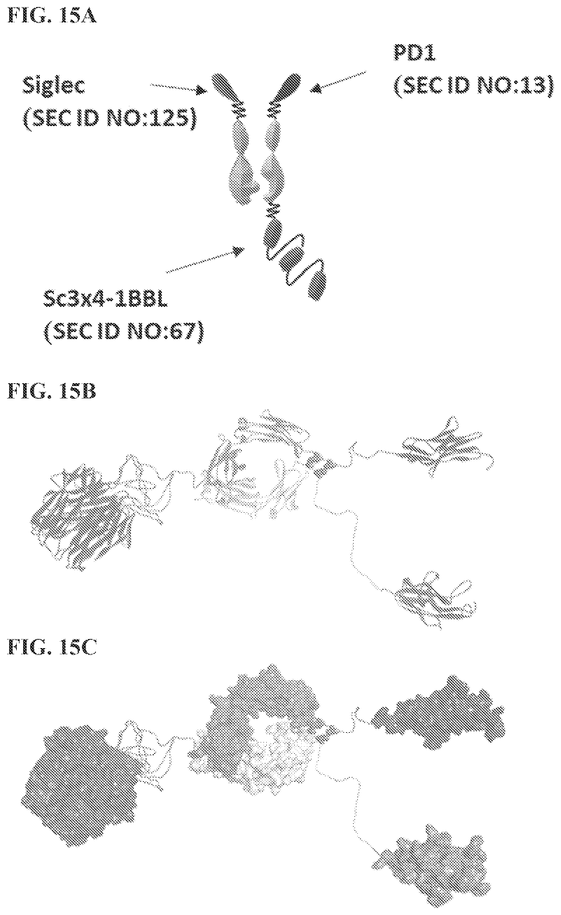

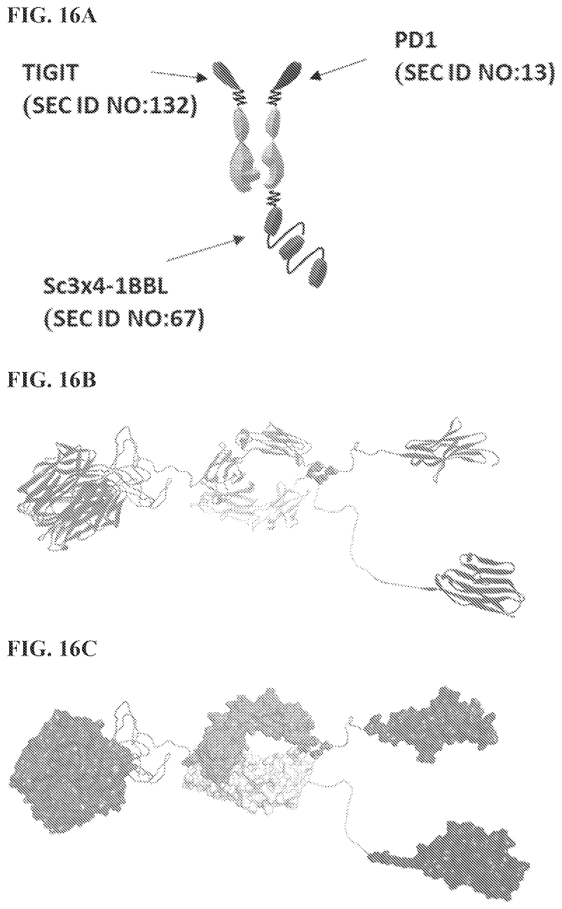

FIGS. 3 and 6 represent 3D models generated for the domains and segments identified of the PD1-Fc-sc3x4-1BBL (FIG. 3) or SIRPα-PD1-Fc-sc3x4-1BBL (FIG. 6) heterodimers, with and without their ligands (CD47, PDL1 and 4-1BB). FIGS. 12A-16C represent schematic schemes of the heterodimers and the 3D models generated for the domains and segments identified of the PD1-SIRPα-Fc-sc3xCD40L (FIGS. 12A-C), LILRB2-Fc-sc3x4-1BBL (FIGS. 13A-C), LILRB2-SIRPα-Fc-sc3xCD40L (FIGS. 14A-C), SIGLEC10-PD1-Fc-sc3x4-1BBL (FIGS. 15A-C) or TIGIT-PD1-Fc-sc3x4-1BBL (FIGS. 16A-C) heterodimers. This analysis predicted possible binding to the ligands and no interference between the different domains. The structural analysis also indicated that an internal (GGGGS)x2+GGGG (SEQ ID NO: 96) linker between the 3 repeats of 4-1BBL amino acid sequence in the sc3x4-1BBL and an internal (GGGGS)x3 (SEQ ID NO: 136 linker between the 3 repeats of CD40L amino acid sequence in the sc3xCD40L would facilitate such binding.

### Example 2

### Manufacturing of Heterodimers

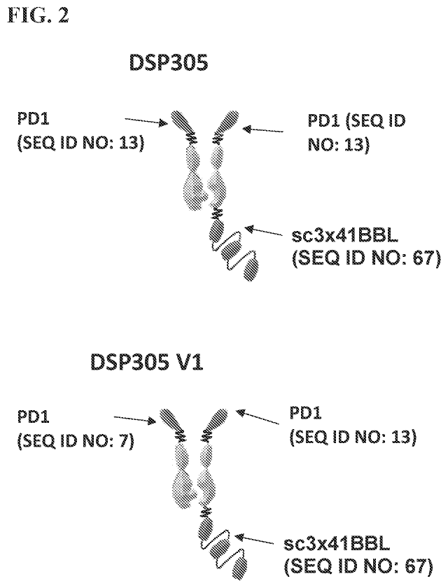

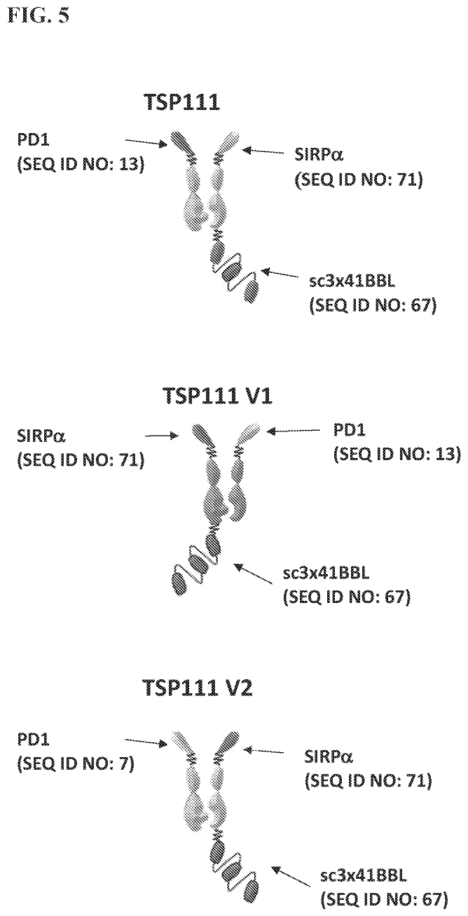

For comparative functional analysis and production evaluation, several heterodimers were produced using the “knob” into “hole” method (described e.g. in U.S. Pat. No. 8,216,805), namely: a PD1-Fc-3xSc4-1BBL heterodimer referred to herein as “DSP305” (SEQ ID NOs: 79 and 81), and “DSP305_V1” (SEQ ID NOs: 79 and 83); PD1-SIRPα-Fc-3xsc4-1BBL heterodimers referred to herein as “TSP111” (SEQ ID NOs: 85 and 81), “TSP111_V1” (SEQ ID NOs: 89 and 91) and “TSP111_V2” (SEQ ID NOs: 85 and 83); a PD1-SIRPα-Fc-3xscCD40L heterodimer referred to herein as “TSP112” (SEQ ID NOs: 81 and 146); LILRB2-PD1-Fc-3xsc4-1BBL heterodimer referred to herein as “DSP214” (SEQ ID NOs: 138 and 142) and “DSP 214 V1” (SEQ ID NOs: 140 and 144); LILRB2-SIRPα-Fc-3xsc4-1BBL heterodimers referred to herein as “TSP215” (SEQ ID NOs: 138 and 85) and “TSP215 V1” (SEQ ID NOs: 140 and 85); a LILRB2-SIRPα-Fc-3xscCD40L heterodimer referred to herein as “TSP217” (SEQ ID NOs: 138 and 146), a 2xLILRB2-Fc-3xscCD40L heterodimer referred to herein as “DSP218” (SEQ ID NOs: 138 and 148); a LILRB2-PD1-Fc-3x4-1BBL heterodimer referred to herein as “TSP221” (SEQ ID NOs: 138 and 79); a LILRB2-PD1-Fc-3xscCD40L heterodimer referred to herein as “TSP222” (SEQ ID NOs: 138 and 154), a SIGLEC10-Fc-PD1-3xsc-4-1BBL heterodimer referred to herein as “TSP401” (SEQ ID NOs: 150 and 79); a SIGLEC10-PD1-Fc-3xscCD40L heterodimer referred to herein as “TSP403” (SEQ ID NOs: 150 and 154), a TIGIT-Fc-PD1-3xsc-4-1BBL heterodimer referred to herein as “TSP501” (SEQ ID NOs: 152 and 79) and a TIGIT-PD1-Fc-3xscCD40L heterodimer referred to herein as “TSP503” (SEQ ID NOs: 152 and 154). Schematic representations of some of the produced heterodimers are shown in FIGS. 2, 5, 12A, 13A, 14A, 15A, 16A and 32.

Production was effected in Expi293F cells transfected by pcDNA3.4 expression vectors cloned with coding sequence for DSP305, DSP305_V1, TSP111, TSP111_V1, TSP111_V2, TSP112, DSP214, DSP214_V1, TSP215, TSP215_V1, TSP217, DSP218, TSP221, TSP222, TSP401, TSP403, TSP501 or TSP503. The sequences were cloned into the vector using EcoRI and HindIII or XbaI and EcoRV restriction enzymes, with addition of Kozak sequence and artificial signal peptide (MESPAQLLFLLLLWLPDGVHA, SEQ ID NO: 95). The proteins were collected from the supernatant of cell culture and in some cases, proteins were purified by one-step purification using protein A (PA) Poros MabCapture A resin or Anion Exchange High Trap Q FF resin.

The production was verified by SDS-PAGE. Specifically, 35 μl supernatant or 3 pg PA-purified protein from each sample were mixed with loading buffer with or without β-mercaptoethanol (reduced and non-reduced conditions, respectively), heated for 5 minutes at 95° C. and separated on 8% or 4-20% gradient polyacrylamide gel electrophoresis SDS-PAGE. Proteins migration on the gel is visualized by e-Stain machinery (GenScript), according to manufacturer instructions.

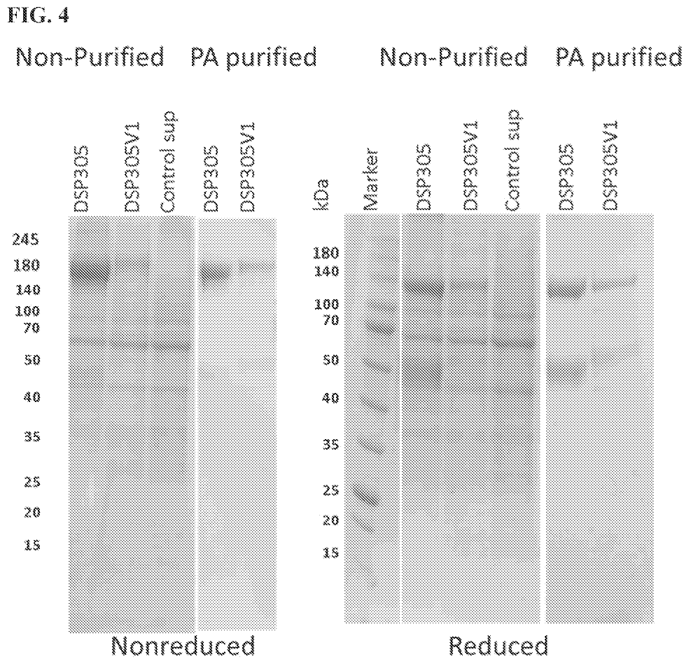

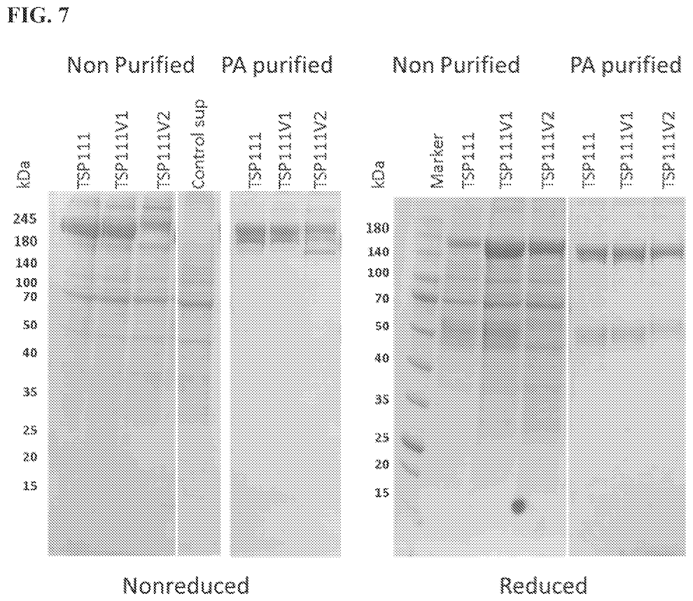

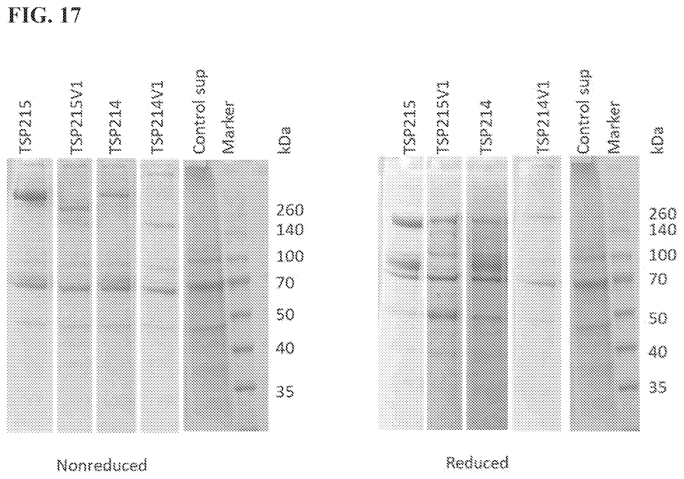

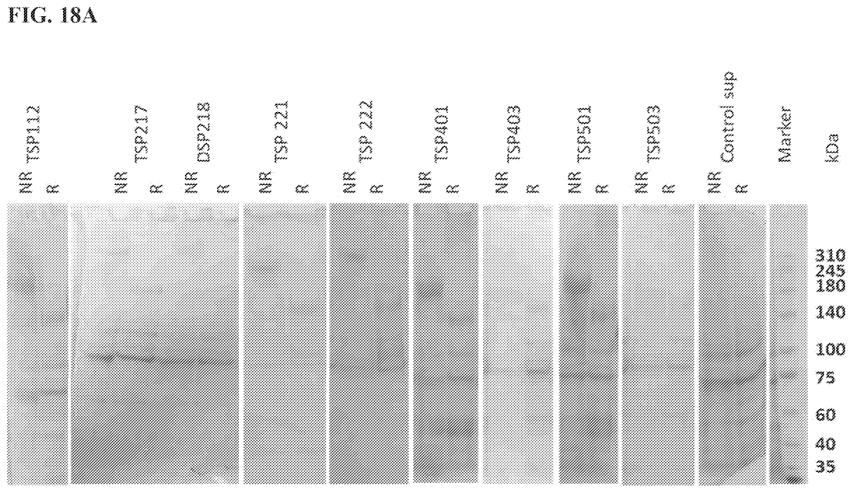

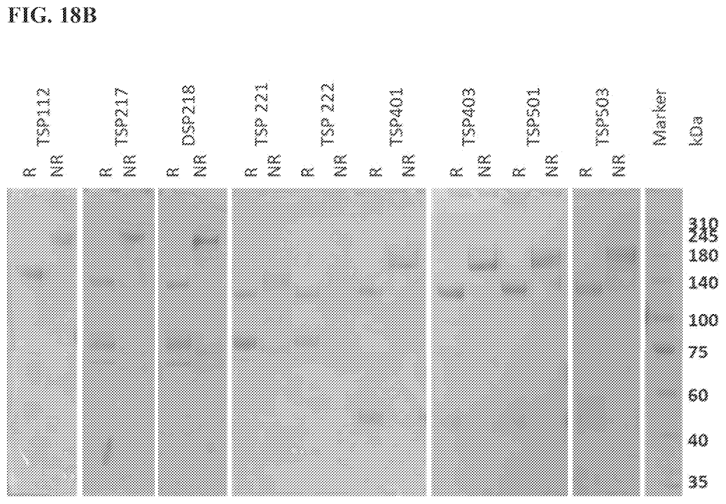

As demonstrated in FIGS. 4, 7 and 17-18B, a high proportion of protein of the expected heterodimer molecular weight form was observed under non-reducing conditions and the expression of the two subunits was confirmed in reducing conditions. Only a minor level of the isomer (dimers comprising two “knob” or two “hole” fragments) was detected by the SDS-PAGE.

In the same manner, several other heterodimers, namely, a PD1-TIGIT-3xsc4-1BBL heterodimer referred to herein as “TSP501V1” (SEQ ID NOs: 81 and 156) and a PD1-TIGIT-3xscCD40L heterodimer referred to herein as “TSP503V1” (SEQ ID NOs: 81 and 158), are produced and analyzed according to the above.

All the produced heterodimers are further analyzed according to the respective Examples 3-13 hereinbelow.

### Example 3

### The Heterodimers Contain all Domains

Materials—heterodimers produced as described in Example 2 hereinabove.

For the Western blot analysis: Spectra BR protein marker (Thermo Fisher Scientific, cat #26634) or 3 color Extra Range protein marker (GenScript, cat # PM2800), Laemmeli Loading buffer (BioRad, cat #161-0747) or sample buffer (GenScript cat # M00676), 8% or 4-20% polyacrylamide gel (BioRad, cat #556-8094 or GenScript cat # M00662 or M00656), anti-human 4-1BBL (BioVision, 5369-100), PD1 (Cell Signaling, cat #86163), anti-human PD1 (GenScript, cat #A01829-40), biotinylated rabbit anti-human SIRPα (LsBio cat # LS-C370337), anti-human LILRB2 (R&D systems cat # MAB2078), anti-human SIRPα (cat # LS-X370337), streptavidin-HRP (Pierce cat # TS21126), anti-human SIGLEC10 (R&D systems cat # AF2130), anti-human TIGIT (MyBioSource cat # MBS9217285, anti-human CD40L (MyBioSource cat # MBS840387), secondary goat anti rabbit IgG (H+L)-HRP conjugate (R&D systems, cat #170-6515), goat anti-mouse IgG HRP-conjugate (Bio-rad cat #170-6516) and ECL Plus Western Blotting substrate (Pierce, cat #32132).

For the sandwich ELISA: Anti 4-1BBL antibody (capture antibody from a matched pair; Abnova #H00008744-AP41) or anti-CD40L, anti PD1-biotinylated antibody, anti-human SIRPα (cat # LS-C370337), anti-human SIGLEC10 (R&D systems cat # AF2130), anti-human TIGIT (MyBioSource cat # MBS9217285, anti-human CD40L (MyBioSource cat # MBS840387), Streptavidin Protein, HRP (#21126, Thermo Scientific), TMB substrate (1-Step™ Ultra TMB-ELISA Substrate Solution, Thermo Scientific #34028).

Methods—

Western blot analysis—The produced heterodimers (50-500 ng per lane) are treated at reducing or non-reducing conditions (in loading buffer with or without β-mercaptoethanol, respectively), heated for 5 minutes at 95° C. and separated on a 8% or 4-20% gradient SDS-PAGE. Following, proteins are transferred onto a PVDF membrane and incubated with primary antibodies for one hour or overnight, anti-human 4-1BBL, anti-PD1, anti-SIRPα, anti-human LILRB2 anti-human SIGLEC10, anti-human TIGIT or anti-human CD40L followed by 1 hour incubation with an HRP-conjugated secondary antibody. Signals are detected following ECL development.

Sandwich ELISA—Plates are coated with anti 4-1BBL or anti CD40L capture antibody (2.5 μg/ml in PBS) and blocked in blocking solution (PBS, 1% BSA, 0.005% Tween). The produced heterodimers, serially diluted in blocking solution, are applied and incubated in coated plates for 2 hours, followed by incubation with a detecting antibody (e.g. anti-PD1 or anti SIRPα biotinylated antibody), and subsequent detection with streptavidin-HRP and TMB substrate, according to manufacturer recommendation. Plates are analyzed using Plate reader (Thermo Scientific, Multiscan FC) at 450 nm, with reference at 620 nm.

Results—

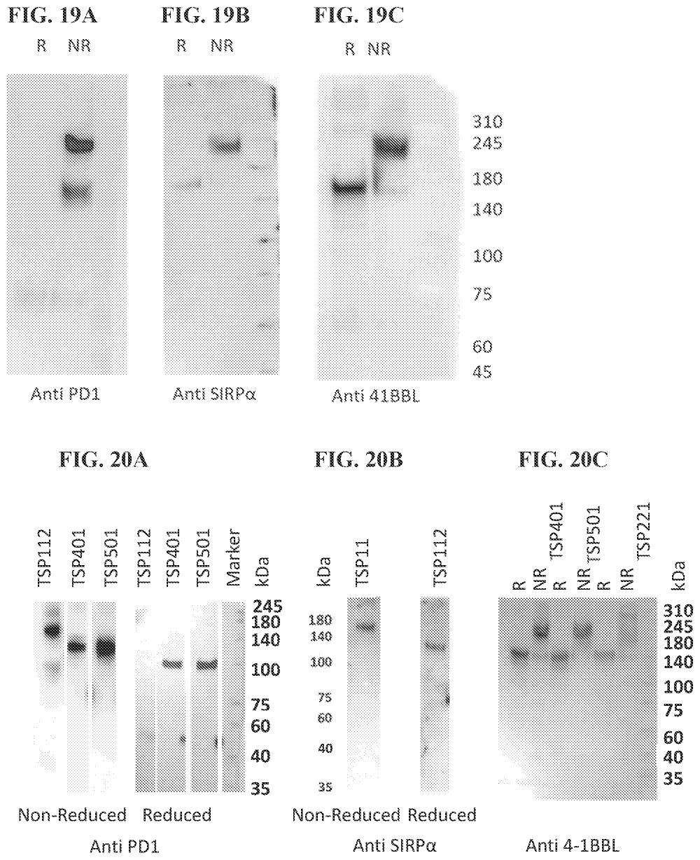

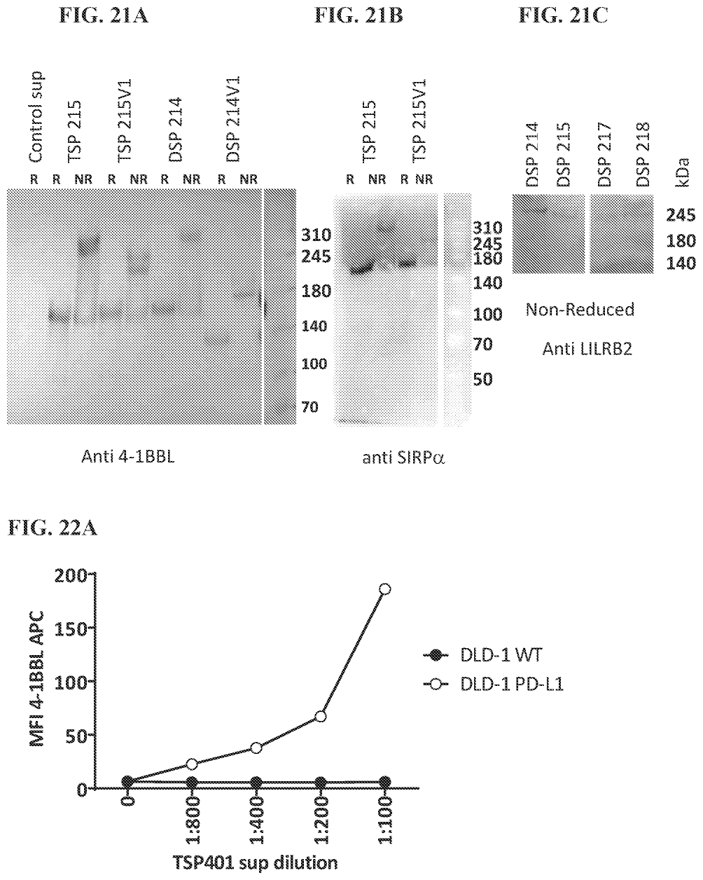

The produced heterodimers contained all their domains, namely: TSP111 contains PD1, SIRPα and 4-1BBL domains (FIGS. 19A-C); TSP112 contains PD1 and SIRPα domains (FIGS. 20A-B); TSP401 contains 4-1BBL and PD1 domains (FIGS. 20A and 20C); TSP501 contains 4-1BBL and PD1 domains (FIGS. 20A and 20C); TSP221 contains a 4-1BBL domain (FIG. 20C); DSP214 contains LILRB2 and 4-1BBL domains (FIGS. 21A and C); DSP214 V1 contains a 4-1BBL domain (FIG. 21A); TSP215 contains LILRB2, SIRPα and 4-1BBL domains (FIGS. 21A-C); TSP215 V1 contains SIRPα, 4-1BBL domains (FIG. 21A-B); TSP217 contains a LILRB2 domain (FIG. 21C); and DSP218 contains a LILRB2 domain (FIG. 21C).

### Example 4

### The Heterodimers Bind their Counterpart-Ligands/Receptors Expressed on Cell's Surface

### Binding Analysis of the PD1 Moiety to PDL1

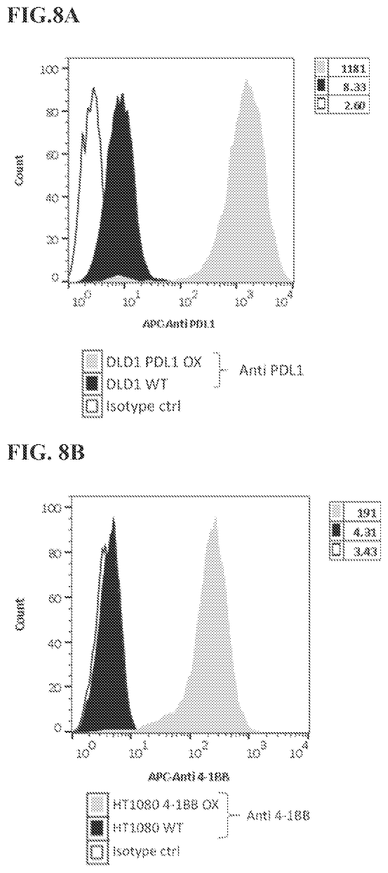

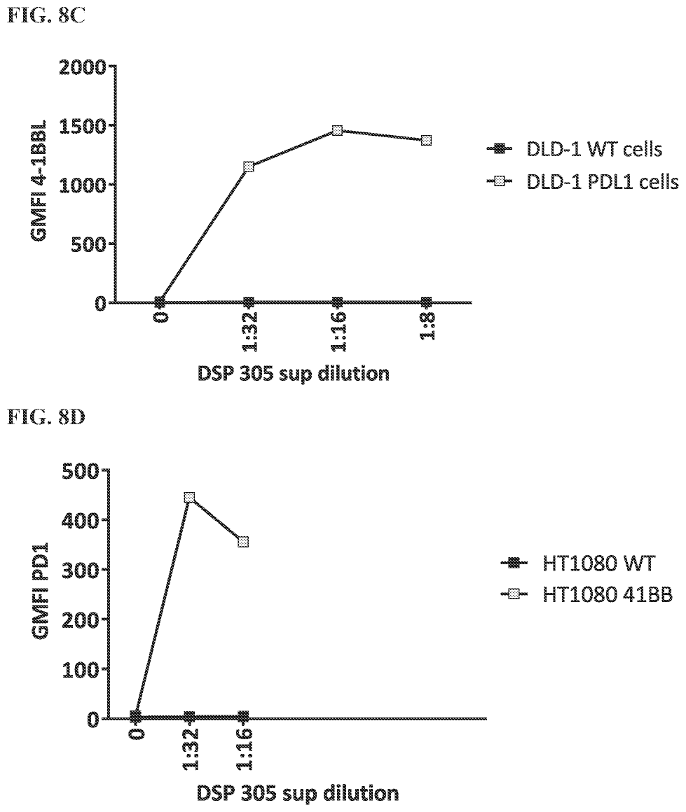

The binding of the PD1 domain of heterodimers comprising a PD1 domain and a 4-1BBL domain (e.g. PD1-4-3xsc1BBL, SIRPα-PD1-3xsc4-1BBL heterodimers) to human PDL1 is determined using DLD1-PDL1 cell line overexpressing PDL1. DLD1-WT cells serve as a control as it expresses low levels of endogenous PDL1 (FIG. 8A). Cells are incubated with different dilutions of supernatant containing the heterodimers, followed by immuno-staining with a conjugated anti 4-1BBL antibody. Binding is analyzed by flow cytometry.

Materials—heterodimers comprising a PD1 domain and a 4-1BBL domain produced as described in Example 2 hereinabove. DLD1-WT and DLD1-PDL1 cell lines (Hendriks et al 2016), anti-human CD47 (InhibRx-like, GenScript), AF647 anti-human CD47 (InhibRx-like, GenScript), APC anti-human CD274 (Biolegend, cat #329708), APC Mouse IgG1, k isotype control (Biolegend, cat #400122), APC anti 4-1BBL (Biolegend, cat #311506).

Methods—Cells were incubated with serial dilutions of the produced heterodimers-containing supernatants for 20 minutes at 4° C., followed by immuno-staining with conjugated antibody against 4-1BBL and analyzed by flow cytometry. In some cases, cells underwent pre-incubation with anti CD47 blocker antibody prior to the incubation with the supernatants.

Results—As shown in FIG. 8A, a high level of membrane expression of PDL1 was observed on DLD1-PDL1 overexpressing cells, compared to DLD1 WT cells that demonstrated low endogenous expression of PDL1.

As shown in FIG. 8C DSP305 (SEQ ID NOs: 79 and 81) bound DLD1 PDL1 overexpressing cells in a dose dependent manner.

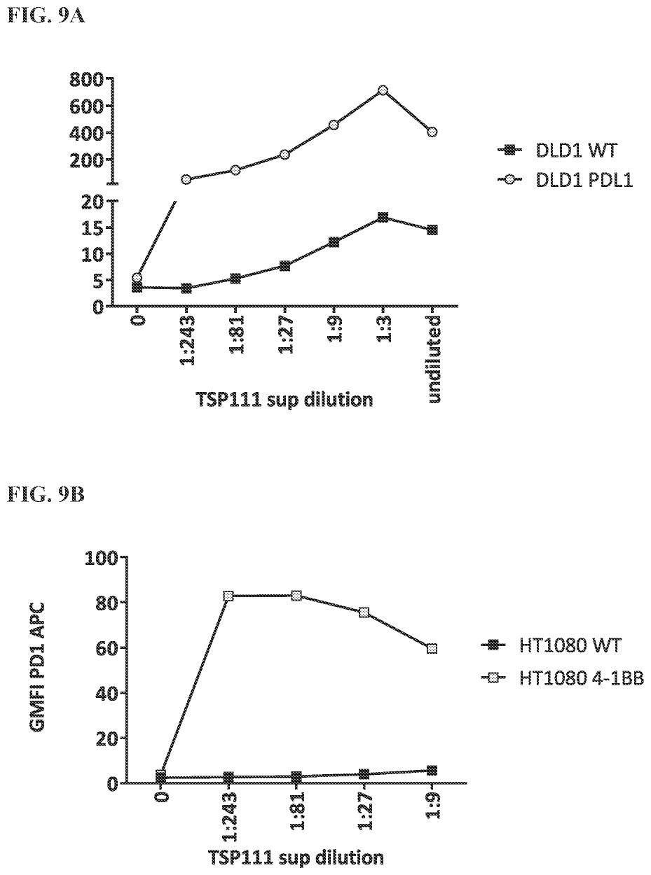

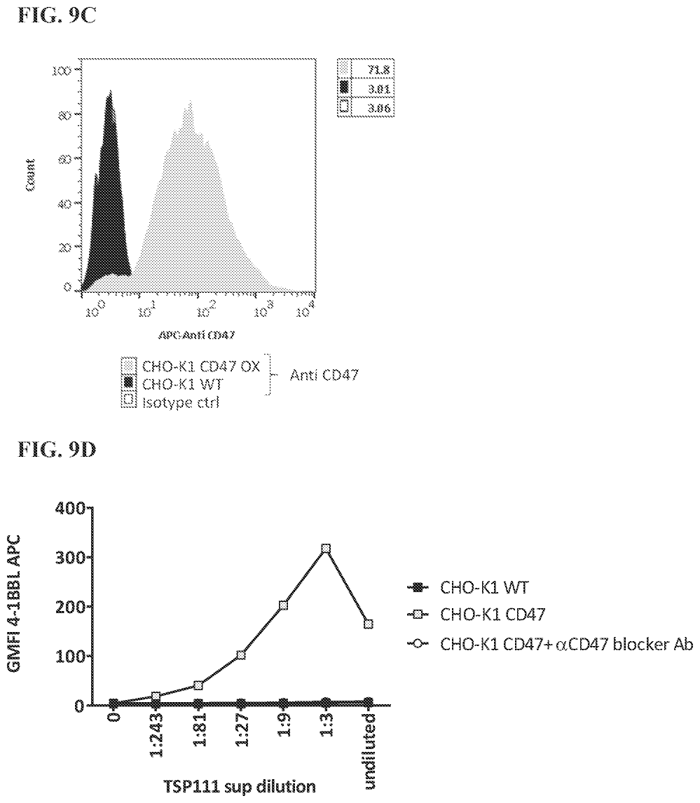

As shown in FIGS. 9A-B, TSP111 (SEQ ID NOs: 85 and 81) bound both DLD1 WT and PDL1-overexpressing cell lines in a dose response manner. As both DLD1 cell lines also express CD47, it is suggested that TSP111's-SIRPα arm bound the DLD1 WT cells mainly through CD47 expressed on the cells, while both TSP111's-PD1 and SIRPα arms bound the DLD1 PDL1-overexpressing cells through PDL1 and CD47 proteins expressed on the cell's surface.

As shown in FIG. 22A TSP401 (SEQ ID NOs: 150 and 79) bound DLD1 PDL1 overexpressing cells in a dose dependent manner.

As shown in FIG. 22B TSP501 (SEQ ID NOs: 152 and 79) bound DLD1 PDL1 overexpressing cells in a dose dependent manner.

### Binding Analysis of the 4-1BBL Moiety to 4-1BB

The binding of the 4-1BBL domains of heterodimers comprising a 4-1BBL domain and a PD1 or LILRB2 domain (e.g. PD1-4-3xsc1BBL and SIRPα-PD1-3xsc4-1BBL heterodimers) to human 4-1BB is determined using a HT1080-4-1BB cell line overexpressing 4-1BB. HT1080 WT cells serve as a negative control. Cells are incubated with different dilutions of supernatants containing the heterodimers, followed by immuno-staining with a conjugated anti-PD1 antibody or anti-LILRB2 antibody. Binding is analyzed by flow cytometry.

Materials—heterodimers comprising a 4-1BBL domain and a PD1 or LILRB2 domain produced as described in Example 2 hereinabove. HT1080 WT and HT1080-4-1BB cells (Wyzgol et al, 2009), anti-human CD47 blocker Ab (InhibRx-like, GenScript), AF647-labeled anti-human CD47, anti-human 4-1BB clone M127 blocker Ab (BD, cat #552532), AF647-labeled anti-human 4-1BB clone M127, APC anti-human CD85d (ILT4) antibody clone 41D1 (Biolegend cat #338708), APC Mouse IgG1, k isotype control (Biolegend, cat #400122), APC-labeled anti PD1 (Biolegend, cat #329908).

Methods—Cells were incubated with serial dilutions of the produced heterodimers-containing supernatants for 20-30 minutes at 4° C., followed by immuno-staining with an antibody against PD1 or LILRB2 and analyzed by flow cytometry. In some cases, cells underwent pre-incubation with anti CD47 blocker antibody or anti-4-1BB blocker antibody prior to the incubation with the supernatants.

Results—As shown in FIG. 8B, membrane expression of 4-1BB was observed on the surface of HT1080 4-1BB cells and not on HT1080 WT cells.

As shown FIG. 8D, DSP305 (SEQ ID NOs: 79 and 81) bound the HT1080 4-1BB overexpressing cells.

As shown in FIG. 9B, TSP111 (SEQ ID NOs: 89 and 91) bound both HT1080-4-1BB cell lines. As both HT1080 cell lines also express CD47, it is suggested that both TSP111's 4-1BBL and/or SIRPα arms bound their receptors (4-1BB, CD47 respectively).

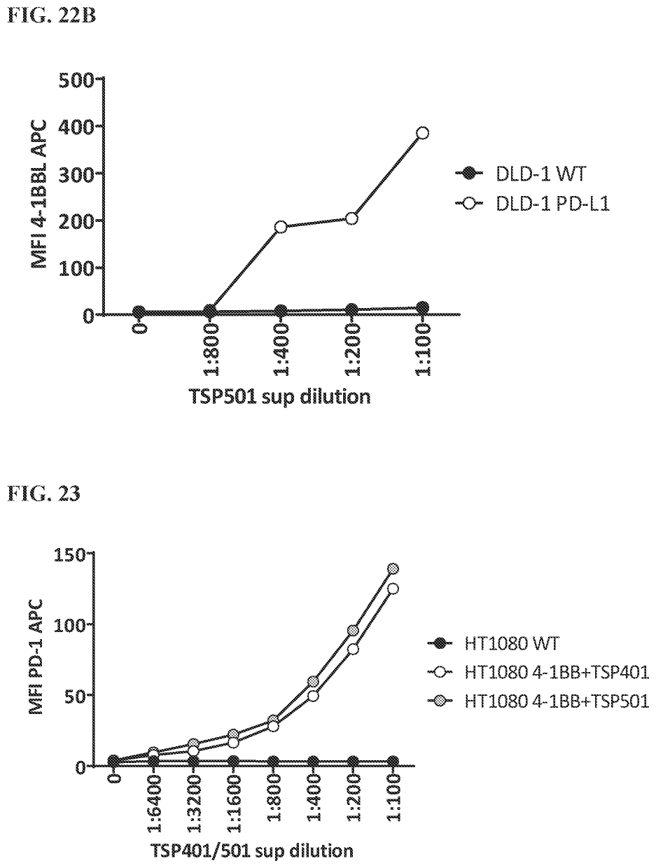

As shown FIG. 23, TSP401 (SEQ ID NOs: 150 and 79) and TSP501 (SEQ ID NOs: 152 and 79) bound the HT1080-4-1BB overexpressing cells.

As shown in FIG. 24, DSP214 (SEQ ID NOs: 138 and 142) bound to HT1080-4-1BB cell line. Pre-incubation with an anti-4-1BB blocker antibody prevented binding completely.

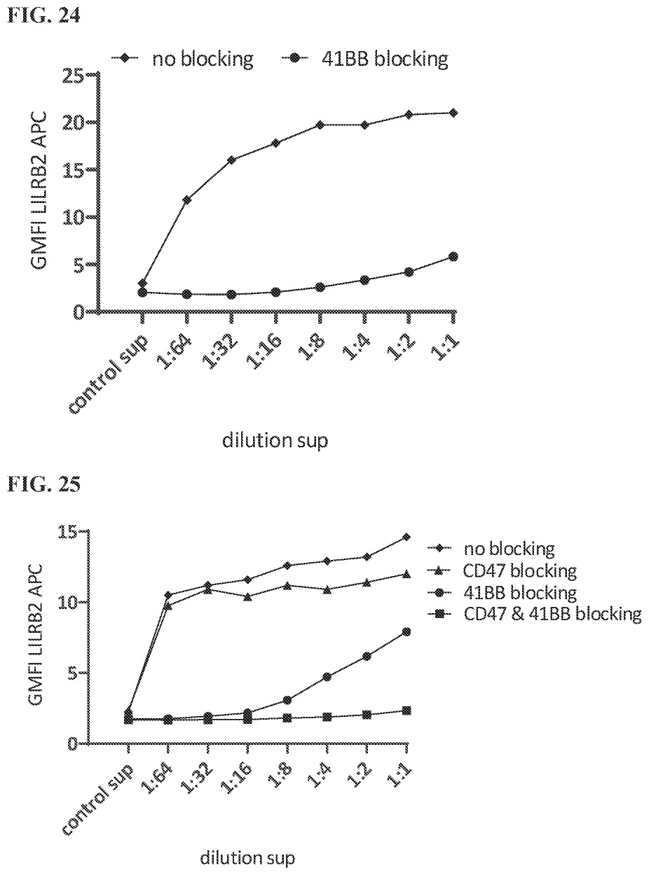

As shown in FIG. 25, TSP215 (SEQ ID NOs: 138 and 85) bound to HT1080-41BB cell line. Pre-incubation with an anti-CD47 blocker antibody or anti-4-1BB blocker antibody reduced binding. Pre-incubation with both anti-CD47 and anti-41BB blocker antibodies, prevented binding completely.

### Binding Analysis of the SIRPα Moiety to CD47

The binding of the SIRPα domain of heterodimers comprising a SIRPα domain and a 4-1BBL domain (e.g. SIRPα-PD1-3xsc4-1BBL heterodimers) to human CD47 is determined using the CHO-K1-CD47 cell line overexpressing human CD47. CHO-K1 WT cells serve as a negative control. Cells are incubated with different dilutions of supernatant containing the heterodimer, followed by immuno-staining with a conjugated anti 4-1BBL antibody. Binding is analyzed by flow cytometry.

Materials—TSP111 (SEQ ID NOs: 85 and 81), TSP111_V1 (SEQ ID NOs: 89 and 91) and TSP111_V2 (SEQ ID NOs: 85 and 83), produced as described in Example 2 hereinabove. CHO-K1 and CHO-K1-CD47 cells, anti-human CD47 blocker Ab (InhibRx-like GenScript), AF647-labeled anti-human CD47, APC-labeled mouse IgG1, k isotype control (Biolegend, cat #400122), APC anti 4-1BBL (Biolegend, cat #311506).

Methods—Cells were incubated with serial dilutions of the produced supernatant containing heterodimers for 20 minutes at 4° C., followed by immuno-staining with antibody against 4-1BBL and analyzed by flow cytometry. In some cases, cells underwent pre-incubation with an anti-CD47 blocker antibody prior to the incubation with the heterodimer.

Results—As shown in FIG. 9C, membrane expression of CD47 was observed on the surface of CHO-K1 CD47 overexpressing cells, compared to CHO-K1 WT cells.

As shown in FIG. 9D, TSP111 (SEQ ID NOs: 85 and 81) bound the CHO-K1-CD47 and not the CHO-K1 WT cells. Pre incubation with an-anti CD47 blocker-antibody completely abolished binding of TSP111 to the CHO-K1-CD47 cells. Taken together, these results indicate binding of the TSP111's SIRPα arm to its ligand (CD47).

### Binding Analysis of the CD40L Moiety to CD40

The binding of the CD40L domain of heterodimers comprising a CD40L domain and a PD1 domain (e.g. SIRPα-PD1-3xscCD40L heterodimer) to human CD40 is determined using a HT1080-CD40 cell line overexpressing CD40. HT1080 WT cells serve as a negative control as they don't express endogenous CD40. Cells are incubated with different dilutions of supernatant containing the heterodimers, followed by immuno-staining with conjugated anti-PD-1 antibody. Binding is analyzed by flow cytometry.

Materials—heterodimers comprising a CD40L domain and a PD1 domain e.g. TSP112 produced as described in Example 2 hereinabove. HT1080 WT and HT1080-CD40 cell line (Wyzgol et al, 2009), APC anti-human CD40 antibody (Biolegend, cat #313008), APC Mouse IgG1, k isotype control (Biolegend, cat #400120), APC-labeled anti-PD1antibody (Biolegend, cat #329908).

Methods—Cells were incubated with serial dilutions of the produced heterodimers-containing supernatants for 20 minutes at 4° C., followed by immuno-staining with conjugated antibody against PD-1 and analyzed by flow cytometry.

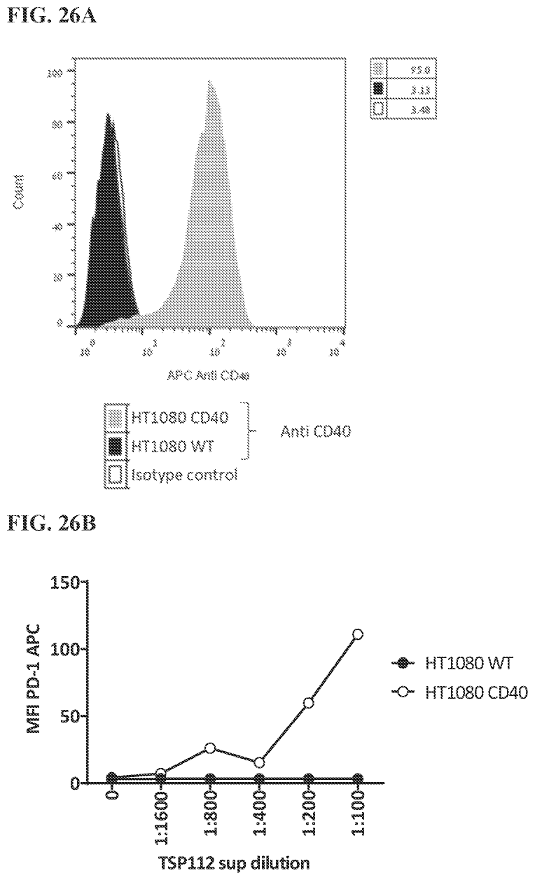

Results—As shown in FIG. 26A, a high level of membrane expression of CD40 was observed on HT1080-CD40 overexpressing cells, compared to HT1080 WT cells that demonstrated no endogenous expression of CD40.

As shown in FIG. 26B TSP112 (SEQ ID NOs: 81 and 146) bound HT1080-CD40 overexpressing cells in a dose dependent manner.

### Binding Analysis of the TIGIT Moiety to CD155 (PVR)

The binding of the TIGIT domain of heterodimers comprising a TIGIT domain and a 4-1BBL domain (e.g. TIGIT-Fc-PD1-3xSc-4-1BBL heterodimer) to human CD155 is determined using a DLD-1 WT cell line endogenously expressing CD155. U937 cells serve as a negative control as they don't express endogenous CD155. Cells are incubated with different dilutions of supernatant containing the heterodimers, followed by immuno-staining with a conjugated anti 4-1BBL antibody. Binding is analyzed by flow cytometry.

Materials—Heterodimers comprising a TIGIT domain and a 4-1BBL domain e.g. TSP501 produced as described in Example 2 hereinabove. DLD1-WT cell line (ATCC, CCL-221), U937 (ATCC, CRL-3253), APC anti-human CD155 antibody (Biolegend, cat #337618), APC Mouse IgG1, k isotype control (Biolegend, cat #400120), APC anti 4-IBBL antibody (Biolegend, cat #311506).

Methods—Cells were incubated with serial dilutions of the produced heterodimers-containing supernatants for 20 minutes at 4° C., followed by immuno-staining with conjugated antibody against 4-1BBL, and analyzed by flow cytometry.

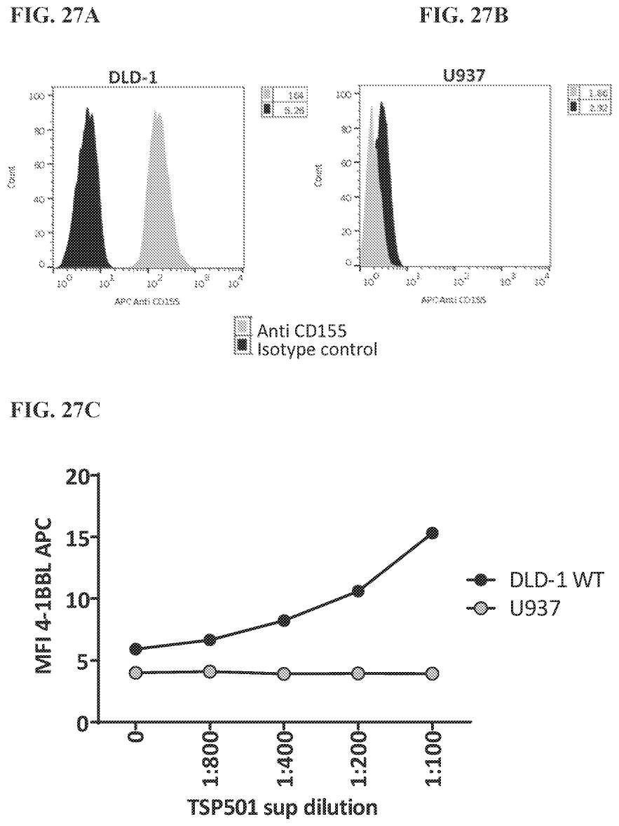

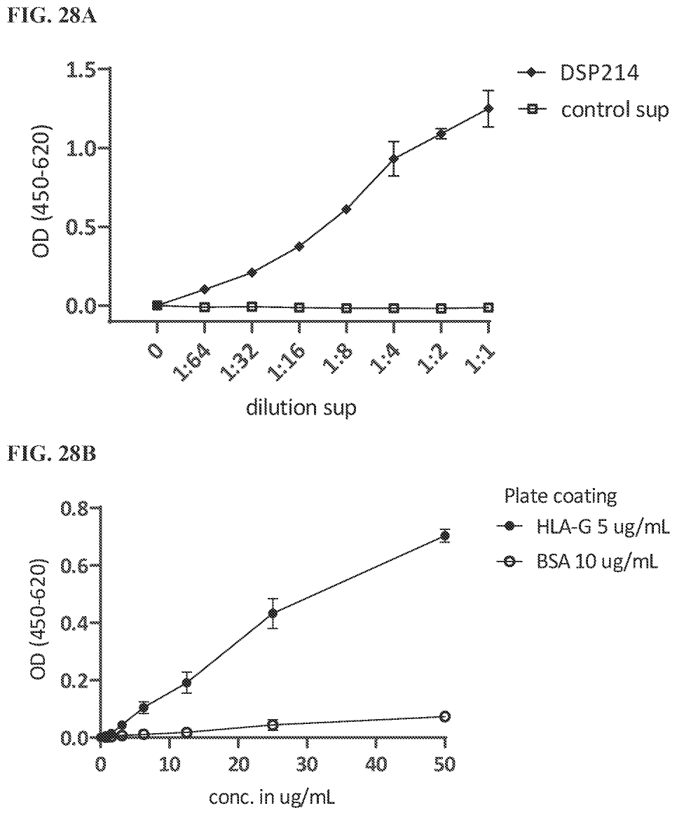

Results—As shown in FIGS. 27A-B, a high level of membrane expression of CD155 was observed on DLD-1 WT cells compared to isotype control antibody, while U937 cells do not express CD155 (FIG. 27B).

As shown in FIG. 27C, TSP501 (SEQ ID NOs: 152 and 79) bound DLD-1 WT cells in a dose dependent manner and did not bind U937 cells.

Binding of the Heterodimers to their Human, Mouse and Cynomolgus Monkey Counterparts

The binding of the heterodimers to their relevant counter-ligands/receptors is determined by Surface Plasmon Resonance (SPR) assays.

Materials—Heterodimers produced as described in Example 2 hereinabove. Series S sensor chip CM5 (GE, cat. # BR100530), Ab capture kit, Negative control protein, human PDL1-hFc (R&D, cat. #156-B7-100), human CD47-hFc (R&D, cat #4670-CD-050), mouse CD47-hFc (R&D, cat. #1866-CD-050), cynomolgus CD47-hFc (ACROBiosystems, cat. # CD7-C5252), human 4-1BB-hFc (LsBio, cat # LS-G4041-100), mouse 4-1BB-hFc (R&D, cat. #937-4B-050), cynomolgus 4-1BB-hFc (R&D, cat. #9324-4B-100), mouse PDL1, cynomolgus PDL1, Methods—SPR assays are performed using Biacore T100 biosensor (GE Healthcare).

Antibody from the capture kit is coupled to all four flow-channels of the chip (Fcl-4), using standard amine coupling protocol as recommended by the manufacturer. As a non-limiting example, for a SIRPα-PD1-3xsc4-1BBL heterodimer, binding of PDL1, CD47 and 4-1BB to the chip is performed in HBS-EP+ running buffer (10 mM HEPES pH7.3, 150 mM NaCl, 3 mM EDTA, 0.05% Tween20): A negative control protein is loaded onto the reference channel Fc1, while Fc2-4 are loaded with the human, mouse and cynomolgus PDL1 proteins. Following automated regeneration of the chip, the chip is re-loaded with a negative control protein on channel Fc1, and with the human, mouse and cynomolgus 4-1BB on channels Fc2-4. Following automated regeneration of the chip, the chip is re-loaded with negative control protein on channel Fc1, and with the human, mouse and cynomolgus CD47 on channels Fc2-4. Following counterparts binding, the SIRPα-PD1-3xsc4-1BBL-variants analytes are passed over all four channels. This process is iteratively repeated with various concentrations of “SIRPα-PD1-3xsc4-1BBL analytes at flow rate of 50 μl/min. 3M MgCl2 solution is injected (45 sec at 20 μl/min) at the end of each cycle, to regenerate the active surface by dislodging the captured molecules. The binding parameters are evaluated using Kinetic 1:1 Binding model in BiaEvaluation software v. 3.0.2 (GE Healthcare). For PD1-3xsc4-1BBL the same procedure applied but in the absence of CD47. In the same manner each heterodimer is studied using the relevant counter-ligands/receptors recombinant proteins from human cynomolgus and mouse origin.

### Example 5

### The Heterodimers Bind their Counterpart Ligands/Receptors Simultaneously

The binding of the heterodimers to their counterpart-ligands/receptors (e.g. the binding of PD1 to PDL1, 4-1BBL to 4-1BB and SIRPα to CD47 in the case of PD1-4-3xsc1BBL and SIRPα-PD1-3xsc4-1BBL heterodimers, the binding of 4-1BBL to 4-1BB, LILRB2 to HLA-G and SIRPα to CD47 in the case of a LILRB2-SIRPα-3xsc4-1BBL heterodimer) is tested by a sandwich ELISA based assay. This assay is also used to compare the functional properties of different variants of the heterodimer proteins.

Materials—Heterodimers produced as described in Example 2 hereinabove. Human recombinant PDL1 (GenScript), human recombinant CD47 (GenScript), human recombinant HLA-G (Abcam), biotin-conjugated human recombinant 4-1BB protein (GenScript), rabbit anti-human SIRPα antibody (anti-drug antibody DSP107), HRP-conjugated streptavidin Protein (cat #21126, Thermoscientific), TMB-ELISA Substrate Solution (Sigma, Cat # T0440) and TMB stop solution (Southern Biotech, cat #0412-01).

Methods—Ninety-six-wells plates were pre-coated by incubating overnight at 4° C. with a recombinant CD47 protein, PDL1 protein, HLA-G protein or a mix of two proteins at equal-molar quantity. Following blocking and washing, serially diluted supernatant containing the produced PD1-4-3xsc1BBL, SIRPα-PD1-3xsc4-1BBL, 2xLILRB2-3xsc4-1BBL or LILRB2-SIRPα-3xsc4-1BBL heterodimers were added to the relevant pre-coated wells. Following an additional washing step, biotinylated 4-1BB or rabbit anti-anti-SIRPα antibody was added and allowed to bind to the 4-1BBL arm or SIRPα arm, respectively, of the heterodimer. The plates were washed again and streptavidin-HRP or goat anti-rabbit IgG antibody-HRP was added. Detection was effected with a TMB substrate according to standard ELISA protocol using a plate reader (Thermo Scientific, Multiscan FC).

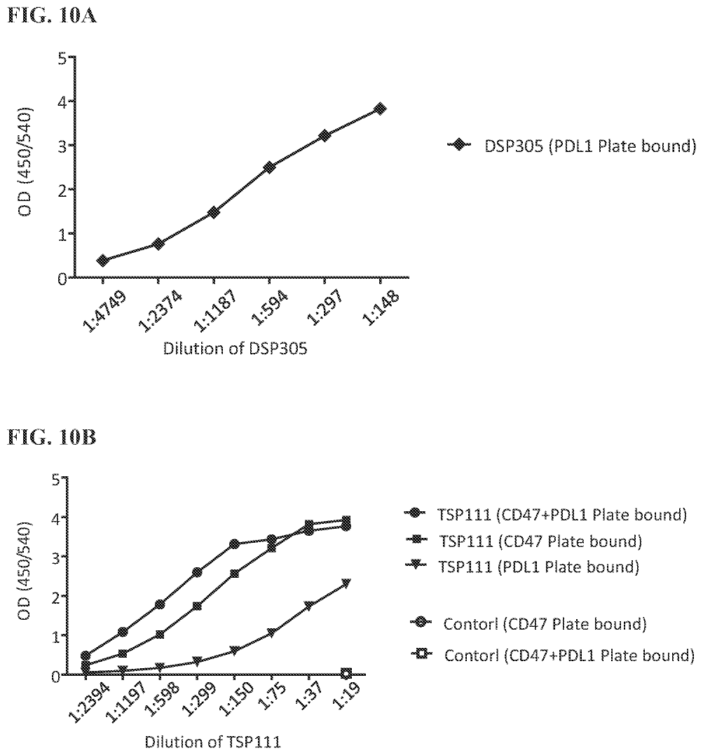

Results—As shown in FIG. 10A, DSP305 (SEQ ID NOs: 79 and 81) was bound the PDL1 coated plate in a concentration dependent manner.

As shown in FIG. 10B, TSP111 (SEQ ID NOs: 85 and 81) bound to both CD47 and PDL1 coated plates in a concentration dependent manner and also bound to plates coated with a mix of CD47 and PDL1. Interestingly, the binding to mixed-proteins coated plates was higher, suggesting stronger binding when both arms (SIRPα and PD1) were involved. The control supernatant did not bind to any of the coated plates.

As shown in 28A, binding of DSP214 (SEQ ID NOs: 138 and 142) to HLA-G was detected via 41BB-biotin in a dose dependent manner, suggesting that the LILRB2 and the 4-1BBL arms were able to bind their targets. No binding was observed with negative control supernatant.

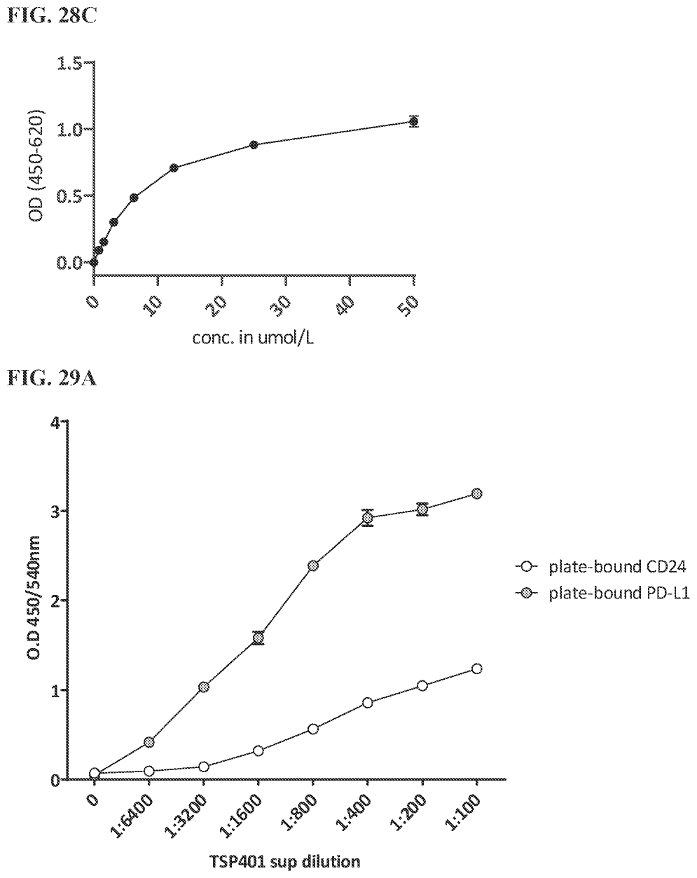

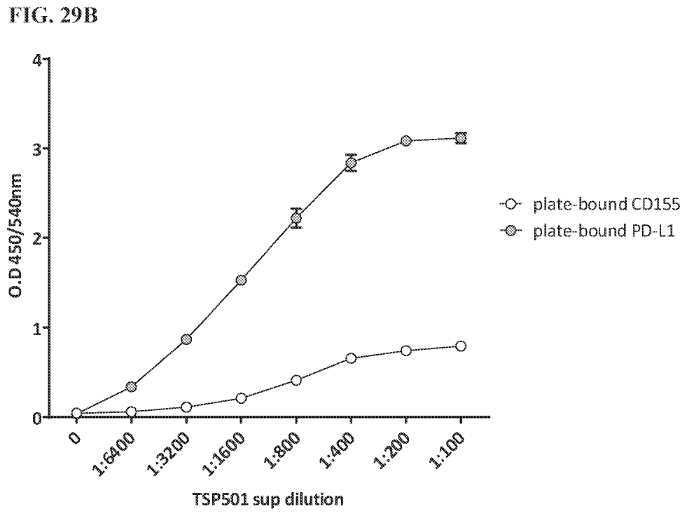

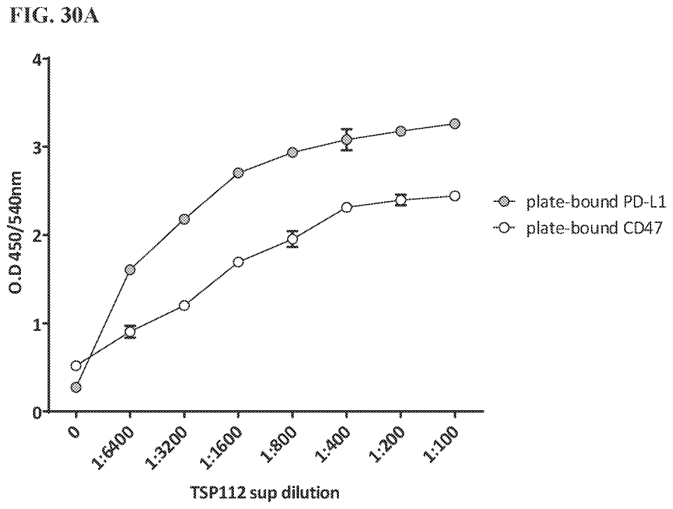

As shown in FIGS. 28B-C, binding of TSP215 (SEQ ID NOs: 138 and 85) to HLA-G or CD47 was detected via 41BB-biotin in a dose dependent manner, suggesting that the LILRB2, SIRPα and 41BBL arms bind their targets. No binding was detected to control plates coated with BSA only.

### Example 6

### Activation of 4-1BB or CD40 by the Heterodimers

### Activation of 4-1BB Receptor

Activation of the 4-1BB receptor-mediated signal transduction by the produced heterodimers comprising a 4-1BBL domain (e.g. PD1-4-3xsc1BBL, SIRPα-PD1-3xsc4-1BBL, SIGLEC10-Fc-PD1-3xSc-4-1BBL and TIGIT-Fc-PD1-3xSc-4-1BBL heterodimers is determined using a 4-BBL overexpressing HT1080 cell-line (HT1080-4-1BB). Upon binding of 4-1BBL to the 4-1BB receptor on the surface of these cells, a signaling pathway is activated, resulting in secretion of IL8 (Wyzgol et al., 2009, The Journal of Immunology). To this end, the cells are incubated overnight in the presence of serially diluted supernatants containing the heterodimers. The incubation is performed in 96-wells plates pre-coated with the relevant proteins e.g. CD47, PDL1, CD24, CD155 or a mix of two relevant proteins in an equal-molar concentration. IL8 secretion from activated HT1080-4-1BB cells to the culture media was determined by ELISA.

Materials—Heterodimers comprising a 4-1BBL domain produced as described in Example 2 hereinabove. Human recombinant PDL1 (GenScript), human recombinant CD47 (GenScript), human recombinant PDL1-Fc tagged (ACRO Biosystem, cat #PD1-H5258), human recombinant CD24-His tagged (ACRO Biosystem, cat #CD4-H52H3), human recombinant CD155 His tagged (ACRO Biosystem, cat #CD5-H5223), HT1080-4-1BB cells, IL-8 ELISA kit (Biolegend, cat #431507), DMEM (Biological industries, cat #01-055-1A), RPMI (Biological industries, cat #01-100-1A), FBS (Gibco, cat #10270106), anti 4-1BB antibody (Biolegend, cat #359810), isotype IgG1, k (Biolegend, cat #400122).

Methods—96-wells plates were pre-coated by incubating overnight at 4° C. with a recombinant CD47 or PDL1 or a mix of both proteins at an equal-molar quantity for DSP305 and TSP111; with PDL1 and CD24 for TSP401; or with PDL1 and CD155 for TSP501. Following a washing step, serially diluted supernatants containing the produced PD1-4-3xsc1BBL, SIRPα-PD1-3xsc4-1BBL, SIGLEC10-Fc-PD1-3xSc-4-1BBL or TIGIT-Fc-PD1-3xSc-4-1BBL heterodimers were added to the relevant pre-coated wells for a 1 hour incubation at 37° C. followed by addition of HT1080 4-1BB cells (10000 per well) for 24 hours at 37° C. Following incubation, IL8 concentration in the supernatant was determined by an IL8 ELISA kit according to the manufacturer's protocol. Supernatants were analyzed for IL-8 concentration using a plate reader (Thermo Scientific, Multiscan FC) at 450 nm, with reference at 540 nm. Expression of 4-1BB receptor on HT1080 4-1BB cells was determined by immuno-staining of cells with the anti-4-1BB antibody and analysis was performed by flow cytometry.

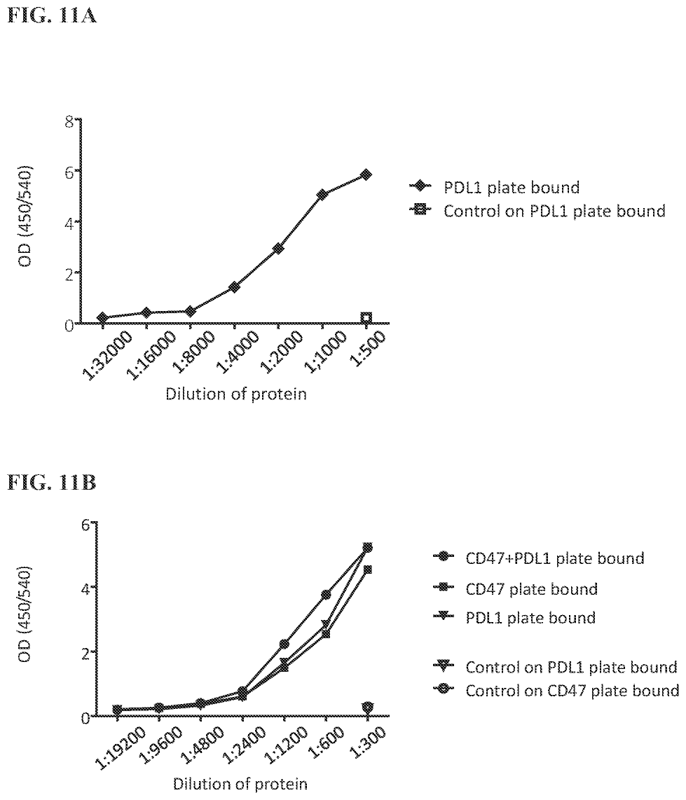

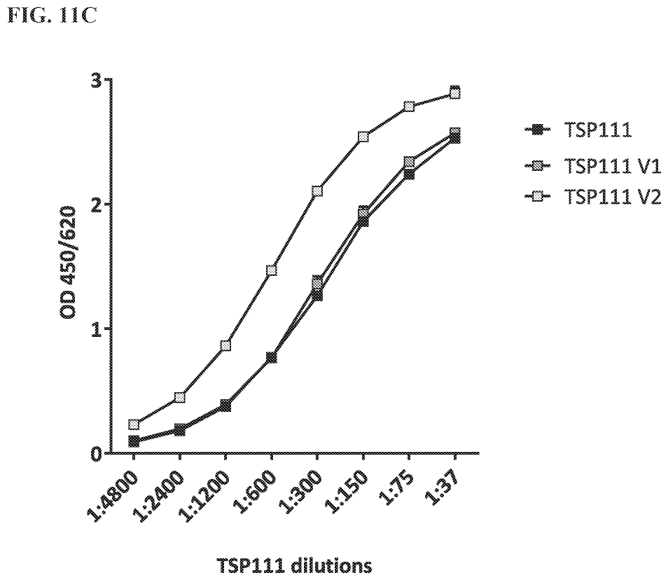

Results—As shown in FIG. 8B, HT1080-4-1BB cells indeed express high levels of the relevant receptor 4-1BB. As shown in FIGS. 11A-C and 29-AB supernatants containing DSP305 SEQ ID NOs: 79 and 81), TSP111 (SEQ ID NOs: 85 and 81), TSP111_V1 (SEQ ID NOs: 89 and 91) or TSP111_V2 (SEQ ID NOs: 85 and 83), TSP401 (SEQ ID NOs: 150 and 79) or TSP501 (SEQ ID NOs: 152 and 79) were able to trigger signaling, in a dose dependent manner, resulting in IL8 secretion from HT1080-41BB cells.

IL8 secretion was not observed when the HT1080-41BB cells were incubated in the presence of control supernatant from non-transfected Expi293F cells.

Interestingly, the level of IL8 secretion was higher, when the cells and the tested SIRPα-PD1-3xsc4-1BBL heterodimers were incubated in plates that were coated with a mixture of CD47 and PD1 recombinant proteins, indicating a stronger binding when both arms (SIRPα and PD1) were involved.

### Activation of CD40 Receptor

Activation of the CD40 receptor-mediated signal transduction by the produced heterodiments comprising a CD40L domain (e.g. PD1-SIRPα-Fc-3xScCD40L, LILRB2-SIRPα-Fc-3xScCD40L and LILRB2-Fc-3xScCD40L heterodimers) is determined using a CD40 overexpressing HT1080 cell line (HT1080-CD40). Upon binding of CD40L to the CD40 receptor on the surface of these cells, a signaling pathway is activated, resulting in secretion of IL8 (Wyzgol et al., 2009, The Journal of Immunology). To this end, the cells are incubated overnight in the presence of serially diluted supernatants containing the heterodimers. The incubation is performed in 96-wells plates pre-coated with the relevant proteins e.g. CD47, PDL1, HLA-G or a combination of two relevant proteins. IL8 secretion from activated HT1080-CD40 cells to the culture media is determined by ELISA.

Materials—Heterodimers produced as described in Example 2 hereinabove. Human recombinant CD47 (ACRO, cat #CD7-H5227), human recombinant PDL1-Fc tagged (ACRO Biosystem, cat #PD1-H5258), human recombinant HLA-G (Abcam, cat #ab225660), HT1080-CD40 cells, IL-8 ELISA kit (Biolegend, cat #431507), DMEM (Biological industries, cat #01-055-1A), RPMI (Biological industries, cat #01-100-1A), FBS (Gibco, cat #10270106), anti-CD40 antibody (Biolegend, cat #313008), isotype IgG1, k (Biolegend, cat #400120).

Methods—96-wells plates were pre-coated by incubating overnight at 4° C. with recombinant CD47 or PDL1 for TSP111; HLA-G or CD47 for TSP217; or HLA-G for DSP218. Following a washing step, serially diluted supernatants containing the produced PD1-SIRPα-Fc-3xScCD40L, LILRB2-SIRPα-Fc-3xScCD40L or LILRB2-Fc-3xScCD40L heterodimers were added to the relevant pre-coated wells for a 1 hour incubation at 37° C. followed by addition of HT1080 CD40 cells (10000 per well) for 24 hours at 37° C. Following incubation, IL8 concentration in the supernatant was determined by an IL8 ELISA kit according to the manufacturer's protocol. Supernatants were analyzed for IL8 concentration using a plate reader (Thermo Scientific, Multiscan FC) at 450 nm, with reference at 540 nm. Expression of CD40 receptor on HT1080 CD40 cells was determined by immuno-staining of cells with the anti-CD40 antibody and analysis was performed by flow cytometry.

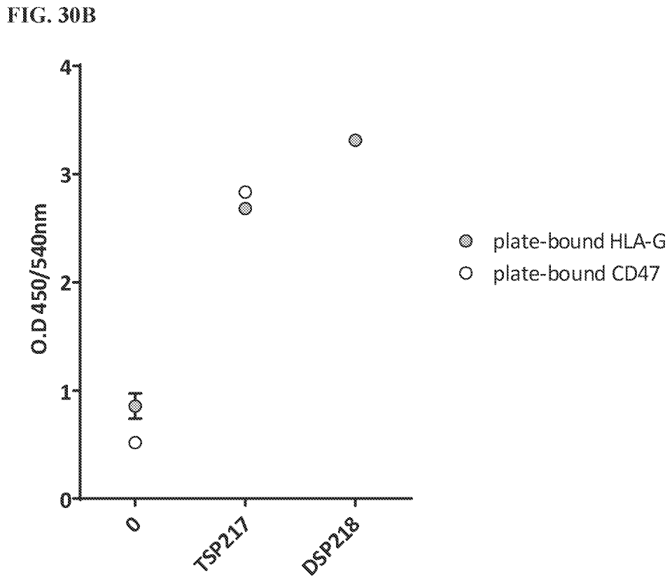

Results— As shown in FIG. 26A, HT1080-CD40 cells indeed express high levels of the relevant receptor CD40. As shown in FIGS. 30A-B, supernatants containing TSP112 (SEQ ID NOs: 81 and 146), TSP217 (SEQ ID NOs: 138 and 146) or DSP218 (SEQ ID NOs: 138 and 148) were able to trigger signaling, in a dose dependent manner, resulting in IL8 secretion from HT1080-CD40 cells.

### Example 7

### The Effect of the Heterodimers on Blocking Ligand-Receptor Binding

The heterodimers are designed to block the interaction of endogenous ligand/receptor expressed on target cells with the native receptor/ligand.

Thus, for example, the PD1 part of the relevant heterodimer (e.g. PD1-4-3xsc1BBL or SIRPα-PD1-3xsc4-1BBL heterodimers) is designed to block the interaction of endogenous PD1 expressed on T cells with PDL1 expressed on tumor cells. To this end, effectiveness of the produced heterodimers as blockers of this interaction is evaluated. Plates (Acro Biosystems, cat. # EP101) are coated overnight with a recombinant human PDL1. Following, plates are washed and incubated for 1 hour with different concentrations of the produced heterodimer (e.g. PD1-4-3xsc1BBL or SIRPα-PD1-3xsc4-1BBL) or the positive control anti-PD1 antibody. Biotinylated PD1 is added followed by additional 1 hour incubation. Following the incubation, the plate is washed and blotted with Streptavidin-HRP and TMB substrate according to standard ELISA protocol. Plates are analyzed using a plate reader (Thermo Scientific, Multiscan FC) at 450 nm, with reference at 620 5 nm.

In a similar manner, the blocking activity of the relevant heterodimers is studied to evaluate their effectiveness to block CD155-TIGIT, SIGLEC10-CD24, LILRB2-HLA-G, CD47-SIRPα, 4-1BBL-4-1BB and/or LILRB2-HLA-G binding.

### Example 8

### Activation of PBMCs or T Cells by Heterodimers Comprising 4-1BBL or CD40L Domain

The activation of a T cell requires two signals: ligation of the T-Cell Receptor (TCR) with the Major Histocompatibility Complex (MHC)/peptide complex on the Antigen Presenting Cell (APC) and cross-linking of co-stimulatory receptors on the T cell with the corresponding ligands on the APC. 4-1BB is a T cell co-stimulatory receptor which upon ligation to 4-1BBL promotes expansion, survival, differentiation and cytokine expression of both CD8+ and CD4+ T cells. CD40 and CD40L are costimulatory molecules that play a pivotal role in the pro-inflammatory immune response. Primarily expressed by activated CD4+ T cells, CD40L binds to CD40 on antigen presenting cells (APCs), thereby inducing APC activation. APCs, in turn, prime cytotoxic T lymphocytes.

Numerous methods are known in the art to determine activation of T cells, including but not limited to:
Expression of activation markers on the surface of the T cells (for example: CD25, CD69, CD62L, CD137, CD107a, PD1 etc.). Expression of activation markers is tested by staining the cells with specific antibodies and flow cytometry analysis (FACS).
Secretion of inflammatory cytokines (for example: IL2, IL6, IL8, INF gamma etc.). Secretion of inflammatory cytokine is tested by ELISA.
Proliferation, measured by pre-staining of T cells with CFSE (carboxyfluorescein succinimidyl ester) or other cell proliferation dyes and determining deviation of cells by CFSE dilution that is determined by FACS. Proliferation is also determined using an Incucyte machine taking photos overtime and analyzing the photos with a specific software.
Killing of a target cell e.g. cancer cell that is measured by pre-labeling the cancer cells using Calcine-AM reagent and measuring Calcine release into the culture medium using luminescence plate reader. Killing is also determined by an Incucyte machine using labeled target cells and caspase sensitive florescent substrate.

### Example 9

### The In-Vivo Anti-Tumor Effect of the Heterodimers

Three different in-vivo mouse models are used for testing the efficacy of the produced heterodimers (e.g. PD1-4-3xsc1BBL and SIRPα-PD1-3xsc4-1BBL) in treating cancer:

1. NSG mice inoculated with human stem cells or with human PBMCs or with immobilized human PBMCs and with human tumor cells. In this model, the heterodimer interacts with the relevant human counter receptor/ligand (e.g. PDL1, expressed on the tumor and the immune cells) and with 4-1BB or CD40 expressed on human immune cells.

2. Nude-SCID mice inoculated with human tumor cells. In this model, the relevant heterodimer interacts with mouse and human CD47 (expressed on the tumor cells) and the effect of the heterodimer on mouse macrophages activity is tested.

3. NSG mice inoculated with human stem cells and human tumor cells. In this model, the heterodimer interacts with the relevant counter-receptor/ligand expressed on the tumor and/or immune cells. For example, for SIRPα-PD1-3xsc4-1BBL, the heterodimer interacts with mouse and human CD47 (expressed on the tumor and the immune cells) and with 4-1BB on human T cells. The effect of the heterodimer on the immune cells and tumor growth is tested.

4. C57BL/6-human-4-1BB knock-in mice inoculated with MC38 mouse colon carcinoma or other cancer cell line or with cancer cell line overexpressing the human relevant counterpart (e.g. PDL1 and/or CD47). In this model, the mouse 4-1BB extracellular domain is replaced by that of a human 4-1BB, hence the heterodimer can interact with the human 4-1BB expressed on mouse T cells. The heterodimer interacts with mouse and with human PDL1 and/or CD47 expressed on the tumor cells.

5. Syngeneic mouse tumor models that expresses the surrogate protein of the tested heterodimer. For example, in this model when testing a heterodimer comprising a PD1 domain, the heterodimer interacts with mouse PDL1 on the tumor cells.

In all models, mice are inoculated with tumor cells intravenously (IV), intraperitoneally (IP), subcutaneously (SC) or orthotopically. Once the tumor is palpable (˜80 mm³), mice are treated IV, IP, SC or orthotopically, with different doses and different regimens of the produced heterodimer (e.g. 4-3xsc1BBL and SIRPα-PD1-3xsc4-1BBL heterodimers).

Mice are followed for weights and clinical signs. Tumors are measured few times a week by a caliper; and tumor volume is calculated according to the following equation: V=length×width²/2. Mice Weight is measured routinely. Tumor growth and survival are monitored through the whole experiment.

Infiltration and sub-typing of immune cells in the tumor is tested by resecting the tumor or draining lymph nodes, digestion and immune phenotyping using specific antibodies staining and flow cytometry analysis. Additionally or alternatively, infiltration of immune cells or necrotic grade of tumors is determined by resecting the tumors, paraffin embedding and sectioning for immunohistochemistry staining with specific antibodies.

At sacrificing, mice organs are harvested and embedded into paraffin blocks for H&E and IHC staining.

Blood samples are taken from mice at different time points, according to common procedures, for the following tests: PK analysis, cytokines measurements in plasma, FACS profiling of blood cells sub-populations in circulation, hematology testing, serum chemistry testing, anti-drug-antibody (ADA) analysis and neutralizing antibodies analysis (NAB).

### Example 10

### Activation of 4-1BB by the LILRB2-SIRPα-3xsc4-1BBL Heterodimer in a HT1080-41BB and CHO-CD47 Cells Co-culture

Materials—TSP215 produced as described in Example 2 hereinabove. HT1080-41BB cells, CHO-K1-CD47 cells, IL-8 ELISA kit (Biolegend, cat #431507), DMEM (Biological industries, cat #01-055-1A), FBS (Rhenium, cat #10270106), APC anti-41BB (Biolegend, cat #309810), APC anti-CD47 antibody (Biolegend, cat #343124), APC isotype IgG1 (Biolegend, cat #400120).

Methods—CHO-K1-CD47 cells were seeded in 96-wells plates. Serially diluted supernatant containing the heterodimer was added to the cells for a 1 hour at 37° C., followed by addition of HT1080-41BB cells and incubation overnight at 37° C. IL8 concentration in the supernatant was determined by an IL8 ELISA kit according to the manufacturer's protocol. Supernatants were analyzed for IL8 concentration using a plate reader (Thermo Scientific, Multiscan FC) at 450 nm, with reference at 540 nm. CD47 expression and expression of 4-1BB receptor and on the cells lines was determined by immuno-staining with anti-CD47 and anti-41BB antibody. Analysis was performed by flow cytometry.

Results— Activation of the 4-1BB receptor-mediated signal transduction by the produced LILRB2-SIRPα-3xsc4-1BBL heterodimer, TSP215, was determined using a 4-BBL overexpressing HT1080 cell-line (HT1080-41BB). Upon binding of 4-1BBL to the 4-1BB receptor on the surface of these cells, a signaling pathway is activated dependent on cross-linking, resulting in secretion of IL8 (Wyzgol et al., 2009, The Journal of Immunology). To provide cross-linking via the SIRPα arm, CHO-K1 cells overexpressing CD47 (CHO-K1-CD47) were seeded in 96-wells plates. Serial dilutions of TSP215 were added on top followed by HT1080-41BB cells. IL8 secretion from activated HT1080-4-1BB cells to the culture media was determined following an overnight co-culture.

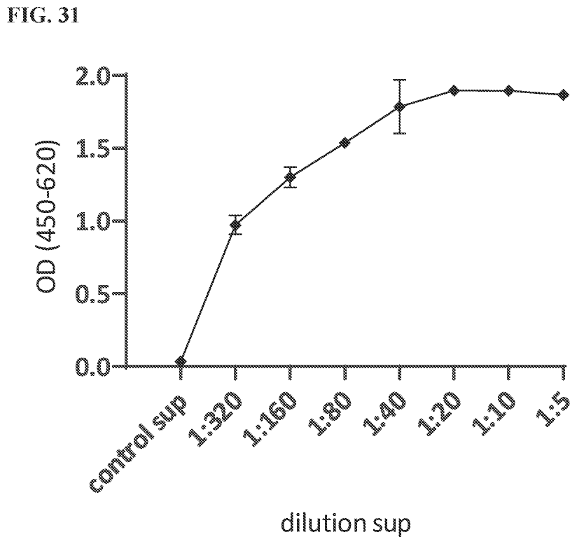

As shown in FIG. 31, TSP215 triggered IL8 release in a dose dependent manner, suggesting that it activates 41BB/41BBL axis following cross-linking via CD47.

### Example 11

### The Effect of the LILRB2 Arm in the Relevant Heterodimers on M-CSF Dependent Macrophage Maturation

The LILRB2 arm of the heterodimers is designed to block the immunosuppressive signals induced by HLA-G expressed on tumor or immune cells towards the endogenous LILRB2 expressed on APCs such as macrophages and dendritic cells, by competing and blocking their interaction. M1-like macrophages show anti-tumor activity, while M2 macrophages have been reported to promote tumor progression. Blocking of LILRB2 with an antagonistic antibody during M-CSF dependent macrophage maturation was shown to lead to a rounder and tightly adherent M1-like (anti-tumor) phenotype with lower expression of CD14 and CD163. After stimulation of the generated macrophages with LPS, enhanced secretion of the pro-inflammatory cytokine TNFα and reduced secretion of anti-inflammatory IL-10 was detected.

To this end, the effect of the produced LILRB2 heterodimers on M-CSF dependent macrophage maturation is evaluated using a flow cytometry-based detection of CD14 and CD163 and by measurement of TNFα and IL-10 release after stimulation of LPS pre-treated macrophages.

Materials—heterodimers produced as described in Example 2 hereinabove, CD14 or CD33 magnetic MicroBeads (Miltenyi Biotec Cat #130-045-501 or Cat #130-045-501), RPMI 1640 25 (Biological Industries, Cat #01-100-1A), FCS (Gibco, Cat #12657-029, M-CSF (R&D systems, Cat #216-MC), TripLE (Thermo Fisher Scientific, Cat #12604-013) LPS (Sigma-Aldrich Cat #L1668-5MG), IL-4 (R&D systems, Cat #204-IL), PE anti-human CD14 antibody (Biolegend, Cat #367104), FITC anti-human CD163 antibody (Biolegend, Cat #333618, IL-10 ELISA (Invitrogen, Cat #88-7106), INFα ELISA (Invitrogen, Cat #88-7346).

Methods—PBMCs are isolated from blood samples of healthy volunteers by density gradient centrifugation, followed by ammonium chloride lysis of erythrocytes.

For the assay, monocytes are further enriched from the isolated PBMCs (e.g. by MACS sorting using CD14 or CD33 magnetic MicroBeads). 20000 monocytes per well are seeded in a 48-wells plate and are differentiated into macrophages (M0) in RPMI 1640 culture medium+10% FCS supplemented with M-CSF (50 ng/ml) in presence or absence of produced heterodimers for 5-7 days. Part of the macrophages are detached with TripLE and stained for CD14 and CD163 expression. The other part is stimulated over night with LPS (50 ng/mL) and IL-4 (25 ng/mL) and release of IL-10 and INF-a to supernatant is measured by ELISA.

### Example 12

### The Effect of the Heterodimers Comprising a Sirpα or Lilrb2 Domain on Macrophages and Polymorphonuclear Cells

As mentioned, the SIRPα part of the heterodimers is designed to block the “don't eat me” signal” induced by CD47 expressing tumor cells, towards the endogenous SIRPα expressed on APCs such as macrophages and granulocytes, by competing and blocking the interaction of CD47 on tumor cells with the endogenous SIRPα. This blockage of the “don't eat me” signal induces tumor cells phagocytosis.

The LILRB2 part of the heterodimer is designed to block the immunosuppressive signals induced by HLA-G expressed on tumor or immune cells towards the endogenous LILRB2 expressed on APCs such as macrophages and DCs, by competing and blocking the interaction of HLA-G on tumor and immune cells with the endogenous LILRB2. This blockage of the HLA-G “don't eat me signal” induces tumor cell phagocytosis and prevents the inhibitory HLA-G-LILRB2 signaling between immune cells, in turn enhancing phagocytosis.

The effect of the produced SIRPα heterodimers on phagocytosis of tumor cells by human macrophages or polymorphonuclear cells (PMNs) and the effect of LILRB2 heterodimers on phagocytosis of tumor cells by human macrophages or DCs are evaluated using a flow cytometry-based assay or fluorescent microscopy.

Materials—heterodimers produced as described in Example 2 hereinabove. CD14 magnetic MicroBeads (Miltenyi Biotec Cat #130-045-501), RPMI 1640 (Biological Industries, Cat #01-100-1A), FCS (Gibco, Cat #12657-029, M-CSF (R&D systems, Cat #216-MC), GM-CSF (R&D systems, Cat #7954-GM/CF, LPS (Sigma-Aldrich Cat #L1668-5MG), INF-7 (MBL, Cat #JM-4116-100), IL-4 (R&D systems, Cat #204-IL), CellTrace™ CFSE Cell Proliferation Kit (Invitrogen, Cat #C34554), PERCP/Cy5.5 anti-human CD11b antibody (Biolegend, Cat #301328), PE Cy7 anti-human HLA-DR antibody (Biolegend, Cat #361708), APC anti-human CD47 antibody (Biolegend, Cat #323124), FITC anti-human HLA-G antibody (Abcam, Cat #ab239334), Rituximab, Cetuximab, human cancer cell lines originated from different cancer types like Lymphoma (e.g. SUDHL6, Ramos) and from solid tumors (e.g. DLD-1—colon carcinoma, A549—lung carcinoma and MDAMB231-triple negative breast cancer), cancer cell lines overexpressing HLA-G and the non-expressing cells as negative controls.

Methods—Polymorphonuclear cells (PMNs) and PBMCs are isolated from blood samples of healthy volunteers by density gradient centrifugation, followed by ammonium chloride lysis of erythrocytes. For the PMNs assay cancer cells are labelled with cell membrane or cytoplasmic dye and mixed with isolated PMN. Mixed cultures are treated with the produced heterodimers, alone or in combination with therapeutic antibodies (e.g. rituximab or cetuximab). Following, phagocytosis of cancer cells by PMNs are analyzed by flow cytometry.

For the macrophages assay, monocytes are further enriched from the isolated PBMCs (e.g. by MACS sorting using CD14 magnetic MicroBeads). Monocytes are differentiated into macrophages (M0) in RPMI 1640 culture medium+10% FCS supplemented with GM-CSF (50 ng/ml) and M-CSF (50 ng/ml) for 7 days. To generate type 1 macrophages (M1), M0 cells are primed by LPS and IFN-γ for additional 24 hours. Monocytes are differentiated for 7 days to monocyte derived DCs in RPMI 1640 culture medium+10% FCS supplemented with 50 ng/mL GM-CSF and 20 ng/mL IL-4. Cancer cells are labelled with cell membrane or cytoplasmic dye and mixed with the isolated and in vitro-differentiated type I macrophages (M1) or DCs. Mixed cultures are treated with the produced heterodimers, alone or in combination with therapeutic antibodies. Following incubation, tumor cells that are not engulfed are washed out and the macrophages are stained with anti-CD11b antibody (M1) or anti-HLA-DR antibody (DCs) with a different color than cancer cells. Phagocytosis of cancer cells by macrophages or DCs are analyzed by flow cytometry. In other experiments, phagocytosis is evaluated by Incucyte as follows: Tumor cells from various cancer cell lines are pre-stained with cytoplasmic dye and macrophages or DCs are stained with anti-human CD11b antibody or anti-HLA-DR antibody, respectively (different color than cytoplasmic dye). Stained tumor cells and macrophages are co-cultured, and images are taken by fluorescence microscope. Phagocytosis is quantified as the proportion of macrophages or DCs positive for tumor cell engulfment (mixed signal) out of the total macrophages (single signal).

### Example 13

### NK Cells Cytotoxic Activity by the Heterodimers Comprising a Tigit Domain

Natural killer (NK) cells induce direct cytotoxicity or secretion of cytokine/chemokine without recognizing a specific antigen as B and T cells. NK cytotoxicity plays an important role in immune response against infected cells, malignancy, and stressed cells, and involves in pathologic process in various diseases.

Numerous assays known in the art are used to determine the effect of the produced heterodimers on NK activation, including but not limited to:
Cytotoxicity assay—Killing of Target cells by NK cells (effector cells) in a co-culture assay. % of killing is analyzed by flow cytometry analysis (FACS). Target cells are placed in 96-wells plates and incubated with pre-labeled primary NK cells at various effector-target (E:T) ratios. NK cells are cultured with 1000 U/mL IL2 for 48 hours before the assay. Following 4 hours and 24 hours, cells are harvested, and assayed by flow cytometry. The numbers of target cells recovered from cultures without NK cells are used as a reference.
Cytotoxicity assay—Killing of Target cells by NK cells (effector cells) in a co-culture assay. % of killing is determined by an Incucyte machine using labeled target cells and caspase sensitive florescent substrate.
Secretion of inflammatory cytokines: primary NK cells are stimulated with various target cells at various ratio for 24 hours. The levels of interferon γ (IFN-γ) and granulocyte-macrophage colony-stimulating factor (GM-CSF) in cell-free culture supernatants are determined with ELISA or Cytometric Bead Array (CBA).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A heterodimer comprising a dimerizing moiety attached to at least one amino acid sequence of at least one type I membrane protein capable of at least binding a natural ligand or receptor of said at least one type I membrane protein and to at least one amino acid sequence of at least one type II membrane protein capable of at least binding a natural ligand or receptor of said at least one type II membrane protein.

2. The heterodimer of claim 1, wherein said dimerizing moiety is a proteinaceous moiety.

3. The heterodimer of claim 1, wherein monomers of said heterodimer are not covalently attached.

4. The heterodimer of claim 1, wherein said dimerizing moiety is an Fc domain of an antibody or a fragment thereof.

5. The heterodimer of claim 1, wherein said at least one type I membrane protein is selected from the group consisting of PD1, SIRPα, LAG3, BTN3A1, CD27, CD80, CD86, ENG, NLGN4X, CD84, TIGIT, CD40, IL-8, IL-10, CD164, LY6G6F, CD28, CTLA4, BTLA, LILRB1, LILRB2, TYROBP, ICOS, VEGFA, CSF1, CSF1R, VEGFB, BMP2, BMP3, GDNF, PDGFC, PDGFD, RAETIE, CD155, CD166, MICA, NRG1, HVEM, DR3, TEK, TGFB1, LY96, CD96, KIT, CD244, GFER and SIGLEC.

6. The heterodimer of claim 1, wherein said at least one type I membrane protein is selected from the group consisting of PD1, SIRPα, TIGIT, LILRB2 and SIGLEC.

7. The heterodimer of claim 1, wherein said at least one type I membrane protein is selected from the group consisting of PD1 and SIRPα.

8. The heterodimer of claim 1, wherein said at least one type II membrane protein is selected from the group consisting of 4-1BBL, FasL, TRAIL, TNF-alpha, TNF-beta, OX40L, CD40L, CD27L, CD30L, RANKL, TWEAK, APRIL, BAFF, LIGHT, VEGI, GITRL, EDA1/2, Lymphotoxin alpha and Lymphotoxin beta.

9. The heterodimer of claim 1, wherein said at least one type II membrane protein is selected from the group consisting of 4-1BBL, OX40L, CD40L, LIGHT and GITRL.

10. The heterodimer of claim 1, wherein said at least one type II membrane protein is selected from the group consisting of 4-1BBL and CD40L.

11. The heterodimer of claim 1, wherein at least one of said type I membrane protein and said type II membrane protein is an immune modulator.

12. The heterodimer of claim 1, wherein said heterodimer comprises a first monomer comprising said at least one amino acid sequence of said at least one type I membrane protein and said at least one amino acid sequence of said at least one type II membrane protein.

13. The heterodimer of claim 1, wherein said heterodimer comprises a first monomer comprising said at least one amino acid sequence of said at least one type II membrane protein and a second monomer comprising said at least one amino acid sequence of said at least one type I membrane protein.

14. The heterodimer of claim 1, wherein said at least one amino acid sequence of said at least one type I membrane protein comprises at least two amino acid sequences of said at least one type I membrane protein; and said heterodimer comprises a first monomer comprising at least one of said at least two amino acid sequences of said at least one type I membrane protein and said at least one amino acid sequence of said at least one type II membrane protein and a second monomer comprising at least one of said at least two amino acid sequences of said at least one type I membrane protein.

15-34. (canceled)

35. A nucleic acid construct or system comprising at least one polynucleotide encoding the heterodimer of claim 2, and a regulatory element for directing expression of said polynucleotide in a host cell.

36. A host cell comprising the heterodimer of claim 2.

37. A method of producing a heterodimer, the method comprising expressing in a host cell a nucleic acid construct or system encoding the heterodimer of claim 1.

38. (canceled)

39. The method of claim 37, comprising isolating the heterodimer.

40. The heterodimer of claim 1, a nucleic acid construct or system encoding same or a cell comprising same for use in treating a disease that can benefit from treatment with said heterodimer.

41-45. (canceled)

46. A method of modulating activity of immune cells, the method comprising in-vitro activating immune cells in the presence of the heterodimer of claim 11, a nucleic acid construct or system encoding same or a host cell comprising same.

47-50. (canceled)


SEQUENCE LISTING (166 sequences)

SEQ ID NO: 1 - protein, 288 aa, Artificial sequence
  aa sequence of full length PD1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - DNA, 867 bp, Artificial sequence
  na sequence of full length PD1
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 3 - protein, 150 aa, Artificial sequence
  sequence of PD1 ECD Full with CYS93>Ser substitution
PGWFLDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLV

SEQ ID NO: 4 - DNA, 450 bp, Artificial sequence
  na sequence encoding SEQ ID NO 3
cccggctggtttctggactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgctggccagtttcagacactcgtg

SEQ ID NO: 5 - protein, 167 aa, Artificial sequence
  PD1 ECM KNOWN FRAGMENT
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 6 - protein, 122 aa, Artificial sequence
  PD1 ECM KNOWN FRAGMENT
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTER

SEQ ID NO: 7 - protein, 150 aa, Artificial sequence [sequence omitted]

SEQ ID NO: 8 - DNA, 450 bp, Artificial sequence
  na pf SEQ ID NO 7
ccaggatggttcttagactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggattgccggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggacagttccaaaccctggtg

SEQ ID NO: 9 - protein, 150 aa, Artificial sequence; identical to SEQ ID NO: 3
  150 aa ORIGINAL PD1 DOMAIN with CYS93>Ser substitution

SEQ ID NO: 10 - DNA, 450 bp, Artificial sequence
  na of SEQ ID NO 9
ccaggatggttcttagactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggattctcggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggacagttccaaaccctggtg

SEQ ID NO: 11 - protein, 140 aa, Artificial sequence
  140 aa -5-5 in PD1 DOMAIN
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 12 - DNA, 419 bp, Artificial sequence
  na of SEQ ID NO 11
gactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggattgccggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggaca

SEQ ID NO: 13 - protein, 140 aa, Artificial sequence
  140 aa -5-5 in PD1 DOMAIN with CYS93>Ser substitution
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 14 - DNA, 419 bp, Artificial sequence
  na of SEQ ID NO 13
gactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggatctccggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggaca

SEQ ID NO: 15 - protein, 140 aa, Artificial sequence; identical to SEQ ID NO: 11
  140 aa PD1 segment

SEQ ID NO: 16 - DNA, 420 bp, Artificial sequence
  na sequence encoding SEQ ID NO 15
gactcccctgacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgccggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgccggccag

SEQ ID NO: 17 - protein, 128 aa, Artificial sequence
  128 aa PD1 segment
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 18 - DNA, 384 bp, Artificial sequence
  na sequence encoding SEQ ID NO 17
ccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgccggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcacct

SEQ ID NO: 19 - protein, 135 aa, Artificial sequence
  135 aa PD1 segment
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 20 - DNA, 405 bp, Artificial sequence
  na sequence encoding SEQ ID NO 19
ccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgccggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgccggccag

SEQ ID NO: 21 - protein, 133 aa, Artificial sequence
  133 aa PD1 segment
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 22 - DNA, 399 bp, Artificial sequence
  na sequence encoding SEQ ID NO 21
gactcccctgacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgccggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcacct

SEQ ID NO: 23 - protein, 135 aa, Artificial sequence
  PD1 -10-5 (with CYS93>Ser substitution)
PWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 24 - DNA, 405 bp, Artificial sequence
  na sequence encoding SEQ ID NO 23
ccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgctggccag

SEQ ID NO: 25 - protein, 128 aa, Artificial sequence
  PD1 -10-12 (with CYS93>Ser substitution)
PWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 26 - DNA, 384 bp, Artificial sequence
  na sequence encoding SEQ ID NO 25
ccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcacct

SEQ ID NO: 27 - protein, 133 aa, Artificial sequence
  PD1 -5-12 (New, from V20) (with CYS93>Ser substitution)
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 28 - DNA, 399 bp, Artificial sequence
  na sequence encoding SEQ ID NO 27
gactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcacct

SEQ ID NO: 29 - protein, 127 aa, Artificial sequence
  PD1 -11-12
WNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 30 - DNA, 381 bp, Artificial sequence
  na sequence encoding SEQ ID NO 29
tggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcacct

SEQ ID NO: 31 - protein, 127 aa, Artificial sequence
  PD1 -11-12 (New, from V18) (with CYS93>Ser substitution)
WNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 32 - DNA, 381 bp, Artificial sequence
  na sequence encoding SEQ ID NO 31
tggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcacct

SEQ ID NO: 33 - protein, 134 aa, Artificial sequence
  PD1 -11-5
WNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 34 - DNA, 402 bp, Artificial sequence
  na sequence encoding SEQ ID NO 33
tggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcaccttctccaagacctgctggccag

SEQ ID NO: 35 - protein, 134 aa, Artificial sequence
  PD1 -11-5 (New, from V19) (with CYS93>Ser substitution)
WNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 36 - DNA, 402 bp, Artificial sequence
  na sequence encoding SEQ ID NO 35
tggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcaccttctccaagacctgctggccag

SEQ ID NO: 37 - protein, 138 aa, Artificial sequence
  PD1 -5-7
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 38 - DNA, 414 bp, Artificial sequence
  na sequence encoding SEQ ID NO 37
gactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcaccttctccaagacctgct

SEQ ID NO: 39 - protein, 138 aa, Artificial sequence
  PD1 -5-7 (New, from V21) (with CYS93>Ser substitution)
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPA

SEQ ID NO: 40 - DNA, 414 bp, Artificial sequence
  na sequence encoding SEQ ID NO 39
gactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactctcggttcagagtgacccagctgcctaacggcagagacttccacatgtccgtcgtgcgggccagaagaaacgactctggcacctatctgtgcggcgccatctctctggctcccaaggctcagatcaaagagtctctgcgggccgagctgagagtgacagaaagacgagctgaggtgcccaccgctcatccctcaccttctccaagacctgct

SEQ ID NO: 41 - protein, 136 aa, Artificial sequence
  PD1 -5-9 from (with out CYS93>Ser substitution)136 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPR

SEQ ID NO: 42 - DNA, 408 bp, Artificial sequence
  na sequence encoding SEQ ID NO 41
gactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaaga

SEQ ID NO: 43 - protein, 145 aa, Artificial sequence
  PD1 -0-5 from (with out CYS93>Ser substitution)145 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 44 - DNA, 435 bp, Artificial sequence
  na sequence encoding SEQ ID NO 43
cccggctggtttctggactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgctggccag

SEQ ID NO: 45 - protein, 143 aa, Artificial sequence
  PD1 -0-7 from (with out CYS93>Ser substitution) 143 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 46 - DNA, 429 bp, Artificial sequence
  na sequence encoding SEQ ID NO 45
cccggctggtttctggactctccagacagaccttggaaccctccaaccttctctcccgctctgctggtggttaccgagggcgacaatgccaccttcacctgttccttcagcaacacctccgagtccttcgtgctgaactggtacagaatgtcccctagcaaccagaccgacaagctggccgcctttcctgaggacagatctcagccaggccaggactgtcggttcagagttacccagctgcctaacggccgggacttccacatgtctgttgtgcgggccagacggaacgactctggcacatatctgtgcggcgccatctctctggctcccaaggctcagatcaaagagtctctgcgggccgagctgagagtgacagaaagacgagctgaggtgcccaccgctcatccctcaccttctccaagacctgct

SEQ ID NO: 47 - protein, 254 aa, Artificial sequence
  aa sequence of full length 41BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - DNA, 765 bp, Artificial sequence
  na sequence of full length 41BBL
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 49 - protein, 205 aa, Artificial sequence
  ORIGINAL 41BBL DOMAIN
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 50 - DNA, 615 bp, Artificial sequence
  na sequence encoding SEQ ID NO: 49
gcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 51 - protein, 191 aa, Artificial sequence
  191 aa 14-0 in 4-1BBL DOMAIN
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 52 - DNA, 573 bp, Artificial sequence
  na of SEQ ID NO 51
agcgccgcctcccccaggctgcgcgagggacctgagctgtccccagacgatcctgccggcctgctggacctgagacagggaatgtttgcccagctggtcgctcagaacgtgctgctgattgacggccccctgtcctggtatagcgatcctggactggcaggcgtgtctctgacaggcggactgagttacaaagaagacactaaagaactggtcgtcgccaaagccggcgtgtactacgtgttcttccaactggagctgaggagggtcgtcgccggcgaaggcagcggctctgtgagcctggccctgcacctgcaaccactgaggagcgccgccggcgccgccgccctggccctgactgtggacctgccaccagcatcctctgaggcaaggaattccgccttcggcttccagggccggctgctgcacctgtctgccggacagagactgggcgtccacctgcataccgaagccagagccaggcatgcctggcagctgacccagggcgccaccgtgctgggcctgttcagagtgaccccagaaattccagcaggactgccttccccaaggtctgag

SEQ ID NO: 53 - protein, 197 aa, Artificial sequence
  197 aa 41BBL segment
ARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 54 - DNA, 591 bp, Artificial sequence
  na sequence encoding SEQ ID NO 53
gctagagcttctcctggctctgccgcttctcccagactgagagaaggacctgagctgagccctgatgatcctgctggactgctggatctgcggcagggcatgtttgctcagttggtggcccagaacgtgctgctgatcgatggccctctgtcctggtactctgatccaggattggctggcgtgtccctgactggcggcctgtcttacaaagaggacaccaaagaactggtggtggccaaggccggcgtgtactacgtgttctttcagctggaactgcggagagtggtggctggcgaaggatctggatctgtgtctctggccctgcatctgcagcctctgagaagtgctgcaggcgctgctgcactggctctgacagttgatctgcctcctgcctcctccgaggccagaaactccgcctttggcttccaaggcagactgctgcacctgtccgctggacagagactgggagtccatctgcacacagaggccagagctagacacgcttggcagttgacacagggcgctacagtgctgggcctgtttagagtgacccctgagattccagccggcctgccatctcctagatctgag

SEQ ID NO: 55 - protein, 185 aa, Artificial sequence
  185 aa 41BBL segment
LREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 56 - DNA, 555 bp, Artificial sequence
  na sequence encoding SEQ ID NO 55
ctgagagaaggacctgagctgagccctgatgatcctgctggactgctggatctgcggcagggcatgtttgctcagttggtggcccagaacgtgctgctgatcgatggccctctgtcctggtactctgatccaggattggctggcgtgtccctgactggcggcctgtcttacaaagaggacaccaaagaactggtggtggccaaggccggcgtgtactacgtgttctttcagctggaactgcggagagtggtggctggcgaaggatctggatctgtgtctctggccctgcatctgcagcctctgagaagtgctgcaggcgctgctgcactggctctgacagttgatctgcctcctgcctcctccgaggccagaaactccgcctttggcttccaaggcagactgctgcacctgtccgctggacagagactgggagtccatctgcacacagaggccagagctagacacgcttggcagttgacacagggcgctacagtgctgggcctgtttagagtgacccctgagattccagccggcctgccatctcctagatctgag

SEQ ID NO: 57 - protein, 199 aa, Artificial sequence
  199 aa 41BBL segment
SGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 58 - DNA, 597 bp, Artificial sequence
  na sequence encoding SEQ ID NO 57
tctggcgctagagcttctcctggctctgccgcttctcccagactgagagaaggacctgagctgagccctgatgatcctgctggactgctggatctgcggcagggcatgtttgctcagttggtggcccagaacgtgctgctgatcgatggccctctgtcctggtactctgatccaggattggctggcgtgtccctgactggcggcctgtcttacaaagaggacaccaaagaactggtggtggccaaggccggcgtgtactacgtgttctttcagctggaactgcggagagtggtggctggcgaaggatctggatctgtgtctctggccctgcatctgcagcctctgagaagtgctgcaggcgctgctgcactggctctgacagttgatctgcctcctgcctcctccgaggccagaaactccgcctttggcttccaaggcagactgctgcacctgtccgctggacagagactgggagtccatctgcacacagaggccagagctagacacgcttggcagttgacacagggcgctacagtgctgggcctgtttagagtgacccctgagattccagccggcctgccatctcctagatctgag

SEQ ID NO: 59 - protein, 184 aa, Artificial sequence
  4-1BBL -21-0  184 aa
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 60 - DNA, 552 bp, Artificial sequence
  na sequence encoding SEQ ID NO 59
agagagggccctgagctgtctcctgatgatcctgctggactgctggacctgagacagggcatgtttgctcagctggtggcccagaacgtgctgctgattgatggccctctgtcctggtactctgatcctggattggctggcgtgtccctgactggcggcctgtcttacaaagaggacaccaaagaactggtggtcgccaaggccggcgtgtactacgtgttctttcagctggaactgcggagagtggtggctggcgaaggatctggatctgtgtctctggccctgcatctgcagcctctgagaagtgctgcaggcgctgctgcactggctctgacagttgatctgcctcctgcctcctccgaggccagaaactccgcctttggcttccaaggcagactgctgcatctgtctgccggacagagactgggagtgcacctccatacagaggccagagctagacacgcttggcagttgacacagggcgctacagtgctgggcctgtttagagtgacacctgagatcccagccggcctgccatctccaagatctgaa

SEQ ID NO: 61 - protein, 182 aa, Artificial sequence
  4-1BBL -23-0 182 aa
GPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 62 - DNA, 546 bp, Artificial sequence
  na sequence encoding SEQ ID NO 61
ggccctgagctgtctcctgatgatcctgctggactgctggacctgagacagggcatgtttgctcagctggtggcccagaacgtgctgctgattgatggccctctgtcctggtactctgatcctggattggctggcgtgtccctgactggcggcctgtcttacaaagaggacaccaaagaactggtggtcgccaaggccggcgtgtactacgtgttctttcagctggaactgcggagagtggtggctggcgaaggatctggatctgtgtctctggccctgcatctgcagcctctgagaagtgctgcaggcgctgctgcactggctctgacagttgatctgcctcctgcctcctccgaggccagaaactccgcctttggcttccaaggcagactgctgcatctgtctgccggacagagactgggagtgcacctccatacagaggccagagctagacacgcttggcagttgacacagggcgctacagtgctgggcctgtttagagtgacacctgagatcccagccggcctgccatctccaagatctgaa

SEQ ID NO: 63 - protein, 183 aa, Artificial sequence
  4-1BBL -14-8 183 aa
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 64 - DNA, 551 bp, Artificial sequence
  na sequence encoding SEQ ID NO 63
gatctgccgcttctcctagactgagagagggccctgagctgtctcctgatgatcctgctggactgctggacctgagacagggcatgtttgctcagctggtggcccagaacgtgctgctgattgatggccctctgtcctggtactctgatcctggattggctggcgtgtccctgactggcggcctgtcttacaaagaggacaccaaagaactggtggtcgccaaggccggcgtgtactacgtgttctttcagctggaactgcggagagtggtggctggcgaaggatctggatctgtgtctctggccctgcatctgcagcctctgagaagtgctgcaggcgctgctgcactggctctgacagttgatctgcctcctgcctcctccgaggccagaaactccgcctttggcttccaaggcagactgctgcatctgtctgccggacagagactgggagtgcacctccatacagaggccagagctagacacgcttggcagttgacacagggcgctacagtgctgggcctgtttagagtgacacctgagatcccagcc

SEQ ID NO: 65 - protein, 176 aa, Artificial sequence
  4-1BBL -21-8  176 aa
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 66 - DNA, 528 bp, Artificial sequence
  na sequence encoding SEQ ID NO 65
agagagggccctgagctgtctcctgatgatcctgctggactgctggacctgagacagggcatgtttgctcagctggtggcccagaacgtgctgctgattgatggccctctgtcctggtactctgatcctggattggctggcgtgtccctgactggcggcctgtcttacaaagaggacaccaaagaactggtggtcgccaaggccggcgtgtactacgtgttctttcagctggaactgcggagagtggtggctggcgaaggatctggatctgtgtctctggccctgcatctgcagcctctgagaagtgctgcaggcgctgctgcactggctctgacagttgatctgcctcctgcctcctccgaggccagaaactccgcctttggcttccaaggcagactgctgcatctgtctgccggacagagactgggagtgcacctccatacagaggccagagctagacacgcttggcagttgacacagggcgctacagtgctgggcctgtttagagtgacacctgagatcccagcc

SEQ ID NO: 67 - protein, 601 aa, Artificial sequence
  601 aa sc3x4-1BBL (-14-0) internal linker (GGGGS)x2+GGGG
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 68 - DNA, 1803 bp, Artificial sequence
  na sequence encoding SEQ ID NO 67
agcgccgcctcccctaggctgcgcgagggaccagagctgagcccagacgatccagcaggactgctggacctgaggcagggaatgttcgcacagctggtggcccagaacgtgctgctgatcgacggccctctgtcttggtacagcgatccaggactggcaggcgtgagcctgacaggcggactgtcctataaggaggataccaaggagctggtggtggcaaaggcaggcgtgtactacgtgttcttccagctggagctgaggagagtggtggcaggagagggatccggatctgtgagcctggccctgcacctgcagccactgcggagcgccgcaggagcagccgccctggccctgaccgtggacctgccacctgcatctagcgaggcacggaattctgccttcggctttcagggcagactgctgcacctgagcgccggacagaggctgggagtgcacctgcacacagaggcaagggcaagacacgcatggcagctgacacagggagcaaccgtgctgggactgttccgcgtgacccctgagatcccagcaggactgccatccccccggtctgagggcggcggcgggtccggaggaggaggatctggcggcggcggcagcgccgcctcccccaggctgcgcgagggacctgagctgtccccagacgatcctgccggcctgctggacctgagacagggaatgtttgcccagctggtcgctcagaacgtgctgctgattgacggccccctgtcctggtatagcgatcctggactggcaggcgtgtctctgacaggcggactgagttacaaagaagacactaaagaactggtcgtcgccaaagccggcgtgtactacgtgttcttccaactggagctgaggagggtcgtcgccggcgaaggcagcggctctgtgagcctggccctgcacctgcaaccactgaggagcgccgccggcgccgccgccctggccctgactgtggacctgccaccagcatcctctgaggcaaggaattccgccttcggcttccagggccggctgctgcacctgtctgccggacagagactgggcgtccacctgcataccgaagccagagccaggcatgcctggcagctgacccagggcgccaccgtgctgggcctgttcagagtgaccccagaaattccagcaggactgccttccccaaggtctgagggaggaggaggaagtggcggaggaggatccggagggggagggagcgccgcctccccaagactgagagagggaccagagctgtcccctgatgaccctgccgggctgctggacctgagacaaggcatgttcgcccagctggtcgcacaaaacgtgctgttaattgacggcccactgtcctggtattccgaccctggcctggccggcgtgtccctgacaggcggcctgtcttacaaagaagacacaaaagaactggtcgtcgctaaagctggcgtgtactacgtgttcttccaattagaactgagaagggtcgtcgccggcgagggcagcgggtctgtgagcctggccctgcacctgcaaccgctgcggagcgccgccggcgctgccgccctggccctgacagtggacctgcctccagcaagctccgaggcaaggaatagcgccttcggctttcaaggccgcctgctgcacctgtccgccggacagcggctgggcgtccacctgcacaccgaagccagagcccgccatgcctggcagctgactcagggcgctaccgtgctgggcctgttccgcgtgaccccagagatccctgccggcctgccctcccctcggtctgag

SEQ ID NO: 69 - protein, 504 aa, Artificial sequence
  aa sequence of full length SIRP
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 70 - DNA, 1512 bp, Artificial sequence
  na sequence of full length SIRP
atggagcccgccggcccggcccccggccgcctcgggccgctgctctgcctgctgctcgccgcgtcctgcgcctggtcaggagtggcgggtgaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtaacatcaccccagcagatgccggcacctactactgtgtgaagttccggaaagggagccctgacacggagtttaagtctggagcaggcactgagctgtctgtgcgtgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtga

SEQ ID NO: 71 - protein, 343 aa, Artificial sequence
  ORIGINAL SIRPa DOMAIN (343AA)
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 72 - DNA, 1029 bp, Artificial sequence
  na sequence encoding SEQ ID NO 71
gaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtaacatcaccccagcagatgccggcacctactactgtgtgaagttccggaaagggagccctgacacggagtttaagtctggagcaggcactgagctgtctgtgcgtgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatatt

SEQ ID NO: 73 - protein, 116 aa, Artificial sequence
  116 aa SIRPa segment
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 74 - DNA, 348 bp, Artificial sequence
  na sequence encoding SEQ ID NO 73
gaagaggaactgcaagtgatccagcctgacaagtccgtgctggtggctgctggcgaaaccgccacactgagatgtaccgccacctctctgatccctgtgggccctatccagtggtttagaggcgctggacctggcagagagctgatctacaaccagaaagagggccactttcctagagtgaccaccgtgtccgacctgaccaagcggaacaacatggacttctccatccggatcggcaacatcacccctgctgatgccggcacctactactgcgtgaagttccggaagggctcccctgacgacgtcgagtttaaatccggcgctggcaccgaactgtccgtgcgagct

SEQ ID NO: 75 - protein, 343 aa, Artificial sequence
  343 amino acids sequence of SIRPa with 4 point mutations
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 76 - DNA, 1029 bp, Artificial sequence
  na sequence encoding SEQ ID NO 75
gaagaggaaatccaagtgatccagcctgacaagtccgtgctggtggctgctggcgaaaccgccacactgagatgtaccatcacctctctgatccctgtgggccctatccagtggtttagaggcgctggacctggcagagtgctgatctacaaccagaaagagggccactttcctagagtgaccaccgtgtccgacctgaccaagcggaacaacatggacttctccatccggatcggcaacatcacccctgctgatgccggcacctactactgcatcaagttccggaagggctcccctgacgacgtcgagtttaaatccggcgctggcaccgaactgtccgtgcgagctaaaccttctgctcccgtggtgtctggccctgccgctagagctacacctcagcacaccgtgtcttttacctgcgagtcccacggcttcagccctagagacatcaccctgaagtggttcaagaacggcaacgagctgtccgacttccagaccaacgtggaccctgtgggagagtccgtgtcctactccatccactctaccgccaaggtggtgctgacccgagaggacgtgcacagccaagtgatctgtgaagtggcccacgtgaccctccagggcgatcctttgagaggcaccgccaacctgtccgagacaatcagagtgcctcctacactggaagtgacccagcagcctgtgcgggccgagaatcaagtgaacgtgacctgccaagtgcggaagttctaccctcagagactgcagctgacctggctggaaaacggcaatgtgtccagaaccgagacagcctccaccgtgaccgagaacaaggatggcacctacaattggatgtcctggctgctcgtgaacgtgtccgctcacagagatgacgtgaagctgacatgccaggtggaacacgatggccagcctgccgtgtctaagtcccacgacctgaaagtgtctgctcaccccaaagagcagggctccaataccgccgctgagaacaccggctccaacgagagaaacatctac

SEQ ID NO: 77 - protein, 116 aa, Artificial sequence
  116 amino acids sequence of SIRPa with 4 point mutations
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRA

SEQ ID NO: 78 - DNA, 348 bp, Artificial sequence
  na sequence encoding SEQ ID NO 77
gaagaggaaatccaagtgatccagcctgacaagtccgtgctggtggctgctggcgaaaccgccacactgagatgtaccatcacctctctgatccctgtgggccctatccagtggtttagaggcgctggacctggcagagtgctgatctacaaccagaaagagggccactttcctagagtgaccaccgtgtccgacctgaccaagcggaacaacatggacttctccatccggatcggcaacatcacccctgctgatgccggcacctactactgcatcaagttccggaagggctcccctgacgacgtcgagtttaaatccggcgctggcaccgaactgtccgtgcgagct

SEQ ID NO: 79 - protein, 989 aa, Artificial sequence
  989 aa hole chain of DSP305 and DSP105_V1
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 80 - DNA, 2967 bp, Artificial sequence
  na sequence encoding SEQ ID NO 79
gactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggattctcggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggacagggaggaggaggatccggaggaggaggatccgagtctaagtatggaccaccatgccctccatgtccagcacctgagtttgagggaggaccttccgtgttcctgtttccccctaagccaaaggacacactgatgatctccaggacaccagaggtgacctgcgtggtggtggacgtgtctcaggaggatcccgaggtgcagttcaactggtacgtggatggcgtggaggtgcacaatgccaagaccaagcctagggaggagcagtttaactccacataccgcgtggtgtctgtgctgaccgtgctgcaccaggattggctgaacggcaaggagtataagtgtaaggtgagcaataagggcctgccaagctccatcgagaagaccatctccaaggcaaagggacagccaagggagcctcaggtgtatacactgccacccagccagtgcgagatgaccaagaaccaggtgagcctgtcctgtgccgtgaagggcttctacccatctgacatcgccgtggagtgggagagcaatggccagcccgagaacaattataagaccacacctccagtgctggactccgatggctctttctttctggtgtccaggctgacagtggataagtctcgctggcaggagggcaacgtgttttcttgtagcgtgatgcacgaggccctgcacaatcactacacccagaagtccctgtctctgagcctgggcaagggcggcggcggctccggaggaggaggaagcgccgcctcccctaggctgcgcgagggaccagagctgagcccagacgatccagcaggactgctggacctgaggcagggaatgttcgcacagctggtggcccagaacgtgctgctgatcgacggccctctgtcttggtacagcgatccaggactggcaggcgtgagcctgacaggcggactgtcctataaggaggataccaaggagctggtggtggcaaaggcaggcgtgtactacgtgttcttccagctggagctgaggagagtggtggcaggagagggatccggatctgtgagcctggccctgcacctgcagccactgcggagcgccgcaggagcagccgccctggccctgaccgtggacctgccacctgcatctagcgaggcacggaattctgccttcggctttcagggcagactgctgcacctgagcgccggacagaggctgggagtgcacctgcacacagaggcaagggcaagacacgcatggcagctgacacagggagcaaccgtgctgggactgttccgcgtgacccctgagatcccagcaggactgccatccccccggtctgagggcggcggcgggtccggaggaggaggatctggcggcggcggcagcgccgcctcccccaggctgcgcgagggacctgagctgtccccagacgatcctgccggcctgctggacctgagacagggaatgtttgcccagctggtcgctcagaacgtgctgctgattgacggccccctgtcctggtatagcgatcctggactggcaggcgtgtctctgacaggcggactgagttacaaagaagacactaaagaactggtcgtcgccaaagccggcgtgtactacgtgttcttccaactggagctgaggagggtcgtcgccggcgaaggcagcggctctgtgagcctggccctgcacctgcaaccactgaggagcgccgccggcgccgccgccctggccctgactgtggacctgccaccagcatcctctgaggcaaggaattccgccttcggcttccagggccggctgctgcacctgtctgccggacagagactgggcgtccacctgcataccgaagccagagccaggcatgcctggcagctgacccagggcgccaccgtgctgggcctgttcagagtgaccccagaaattccagcaggactgccttccccaaggtctgagggaggaggaggaagtggcggaggaggatccggagggggagggagcgccgcctccccaagactgagagagggaccagagctgtcccctgatgaccctgccgggctgctggacctgagacaaggcatgttcgcccagctggtcgcacaaaacgtgctgttaattgacggcccactgtcctggtattccgaccctggcctggccggcgtgtccctgacaggcggcctgtcttacaaagaagacacaaaagaactggtcgtcgctaaagctggcgtgtactacgtgttcttccaattagaactgagaagggtcgtcgccggcgagggcagcgggtctgtgagcctggccctgcacctgcaaccgctgcggagcgccgccggcgctgccgccctggccctgacagtggacctgcctccagcaagctccgaggcaaggaatagcgccttcggctttcaaggccgcctgctgcacctgtccgccggacagcggctgggcgtccacctgcacaccgaagccagagcccgccatgcctggcagctgactcagggcgctaccgtgctgggcctgttccgcgtgaccccagagatccctgccggcctgccctcccctcggtctgag

SEQ ID NO: 81 - protein, 379 aa, Artificial sequence
  379 aa knob chain of DSP305
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - DNA, 1137 bp, Artificial sequence
  na sequence encoding SEQ ID NO 81
gactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgctcccagccaggccaggattctcggttcagagtgacccagctgccaaatggcagggactttcacatgagcgtggtgcgcgcccggagaaacgattccggcacatacctgtgcggagcaatctctctggcaccaaaggcacagatcaaggagtccctgagggcagagctgagggtgaccgagaggagggccgaggtgccaacagcacacccatctcctagcccaaggccagcaggacagggcggaggaggcagcggaggaggaggatccgagtctaagtacggaccaccatgccctccatgtcctgcaccagagttcgagggaggaccatccgtgttcctgtttccacctaagcctaaggacaccctgatgatctccagaacccccgaggtgacatgcgtggtggtggacgtgtctcaggaggatcctgaggtgcagttcaattggtacgtggatggcgtggaggtgcacaacgccaagacaaagccccgggaggagcagtttaatagcacctacagagtggtgtccgtgctgacagtgctgcaccaggattggctgaatggcaaggagtataagtgtaaggtgagcaacaagggcctgcctagctccatcgagaagaccatctccaaggccaagggccagccaagagagccacaggtgtgcaccctgccaccaagccaggaggagatgacaaagaatcaggtgtccctgtggtgtctggtgaagggcttctacccttccgacatcgccgtggagtgggagtctaacggccagccagagaacaattacaagaccacacctccagtgctggactctgatggcagcttctttctgtattctcggctgaccgtggataagagcagatggcaggagggcaacgtgttcagctgctccgtgatgcacgaggccctgcacaaccactatacacagaagtctctgagcctgtccctgggcaag

SEQ ID NO: 83 - protein, 389 aa, Artificial sequence
  389 aa knob chain of DSP305_V1
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - DNA, 1167 bp, Artificial sequence
  na sequence encoding SEQ ID NO 83
ccaggatggttcctggacagccccgataggccttggaatccccctaccttttcccctgccctgctggtggtgacagagggcgacaacgccaccttcacatgctcttttagcaacacctccgagtctttcgtgctgaattggtacaggatgagcccttccaaccagacagataagctggcagcatttccagaggaccgcagccagcctggacaggattgccggttcagagtgacccagctgccaaatggcagggactttcacatgtccgtggtgcgcgcccggagaaacgattctggcacatacctgtgcggagcaatcagcctggcaccaaaggcacagatcaaggagtccctgagggcagagctgagggtgaccgagaggagggccgaggtgccaacagcacacccatctcctagcccacggcccgccggccagttccagaccctggtgggaggaggaggaagcggaggaggaggatccgagtctaagtacggaccaccatgccctccatgtcctgcaccagagtttgagggcggcccatccgtgttcctgtttccccctaagcccaaggacaccctgatgatcagccggaccccagaggtgacatgcgtggtggtggacgtgagccaggaggatcctgaggtgcagttcaattggtacgtggatggcgtggaggtgcacaacgccaagacaaagccccgggaggagcagtttaattccacctacagagtggtgtctgtgctgacagtgctgcaccaggattggctgaatggcaaggagtataagtgtaaggtgtccaacaagggcctgcccagctccatcgagaagaccatctctaaggccaagggccagccaagagagccacaggtgtgcaccctgccaccatcccaggaggagatgacaaagaatcaggtgtctctgtggtgtctggtgaagggcttctacccttctgacatcgcagtggagtgggagagcaacggacagccagagaacaattacaagaccacacctccagtgctggactctgatggcagcttctttctgtatagccggctgaccgtggataagtccagatggcaggagggcaacgtgttcagctgttccgtgatgcacgaggccctgcacaaccactatacacagaagtctctgagcctgtccctgggcaag

SEQ ID NO: 85 - protein, 1192 aa, Artificial sequence
  1192 aa TSP111 and TSP111_V2 “hole”  chain
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 86 - DNA, 3577 bp, Artificial sequence
  na of SEQ ID NO 85
gaggaggagctgcaggtcatccagcccgataagtctgtgctggtggcagcaggagagaccgccacactgagatgcaccgccacaagcctgatcccagtgggaccaatccagtggtttaggggagcaggacctggaagggagctgatctacaaccagaaggagggccacttcccaagggtgaccacagtgtccgacctgaccaagcggaacaatatggatttttctatcagaatcggcaatatcacacctgccgacgccggcacctactattgcgtgaagttcagaaagggcagcccagacgatgtggagtttaagagcggagcaggaaccgagctgtccgtgagagccaagccttctgccccagtggtgagcggaccagcagcaagggcaaccccacagcacacagtgtccttcacctgtgagagccacggcttttccccacgcgatatcacactgaagtggttcaagaacggcaatgagctgagcgactttcagaccaacgtggatcccgtgggcgagtctgtgagctactccatccactctacagccaaggtggtgctgacccgggaggacgtgcacagccaggtcatctgcgaggtggcacacgtgaccctgcagggcgatcctctgagaggcacagccaatctgagcgagaccatcagggtgccccctacactggaggtgacccagcagcccgtgagggcagagaaccaagtgaatgtgacatgtcaggtgcggaagttctaccctcagagactgcagctgacctggctggagaacggcaacgtgagccggaccgagacagccagcaccgtgacagagaacaaggacggcacatataattggatgtcttggctgctggtgaacgtgagcgcccacagggacgatgtgaagctgacctgccaggtggagcacgacggacagccagccgtgtctaagagccacgatctgaaggtgagcgcccaccctaaggagcagggctccaacacagccgccgagaataccggcagcaacgagagaaatatctacggaggaggaggatccggaggaggaggatccgagtctaagtatggaccaccatgccctccatgtccagcacctgagtttgagggaggaccttccgtgttcctgtttccccctaagccaaaggacacactgatgatctccaggacaccagaggtgacctgcgtggtggtggacgtgtctcaggaggatcccgaggtgcagttcaactggtacgtggatggcgtggaggtgcacaatgccaagaccaagcctagggaggagcagtttaactccacataccgcgtggtgtctgtgctgaccgtgctgcaccaggattggctgaacggcaaggagtataagtgtaaggtgagcaataagggcctgccaagctccatcgagaagaccatctccaaggcaaagggacagccaagggagcctcaggtgtatacactgccacccagccagtgcgagatgaccaagaaccaggtgagcctgtcctgtgccgtgaagggcttctacccatctgacatcgccgtggagtgggagagcaatggccagcccgagaacaattataagaccacacctccagtgctggactccgatggctctttctttctggtgtccaggctgacagtggataagtctcgctggcaggagggcaacgtgttttcttgtagcgtgatgcacgaggccctgcacaatcactacacccagaagtccctgtctctgagcctgggcaagggcggcggcggctccggaggaggaggaagcgccgcctcccctaggctgcgcgagggaccagagctgagcccagacgatccagcaggactgctggacctgaggcagggaatgttcgcacagctggtggcccagaacgtgctgctgatcgacggccctctgtcttggtacagcgatccaggactggcaggcgtgagcctgacaggcggactgtcctataaggaggataccaaggagctggtggtggcaaaggcaggcgtgtactacgtgttcttccagctggagctgaggagagtggtggcaggagagggatccggatctgtgagcctggccctgcacctgcagccactgcggagcgccgcaggagcagccgccctggccctgaccgtggacctgccacctgcatctagcgaggcacggaattctgccttcggctttcagggcagactgctgcacctgagcgccggacagaggctgggagtgcacctgcacacagaggcaagggcaagacacgcatggcagctgacacagggagcaaccgtgctgggactgttccgcgtgacccctgagatcccagcaggactgccatccccccggtctgagggcggcggcgggtccggaggaggaggatctggcggcggcggcagcgccgcctcccccaggctgcgcgagggacctgagctgtccccagacgatcctgccggcctgctggacctgagacagggaatgtttgcccagctggtcgctcagaacgtgctgctgattgacggccccctgtcctggtatagcgatcctggactggcaggcgtgtctctgacaggcggactgagttacaaagaagacactaaagaactggtcgtcgccaaagccggcgtgtactacgtgttcttccaactggagctgaggagggtcgtcgccggcgaaggcagcggctctgtgagcctggccctgcacctgcaaccactgaggagcgccgccggcgccgccgccctggccctgactgtggacctgccaccagcatcctctgaggcaaggaattccgccttcggcttccagggccggctgctgcacctgtctgccggacagagactgggcgtccacctgcataccgaagccagagccaggcatgcctggcagctgacccagggcgccaccgtgctgggcctgttcagagtgaccccagaaattccagcaggactgccttccccaaggtctgagggaggaggaggaagtggcggaggaggatccggagggggagggagcgccgcctccccaagactgagagagggaccagagctgtcccctgatgaccctgccgggctgctggacctgagacaaggcatgttcgcccagctggtcgcacaaaacgtgctgttaattgacggcccactgtcctggtattccgaccctggcctggccggcgtgtccctgacaggcggcctgtcttacaaagaagacacaaaagaactggtcgtcgctaaagctggcgtgtactacgtgttcttccaattagaactgagaagggtcgtcgccggcgagggcagcgggtctgtgagcctggccctgcacctgcaaccgctgcggagcgccgccggcgctgccgccctggccctgacagtggacctgcctccagcaagctccgaggcaaggaatagcgccttcggctttcaaggccgcctgctgcacctgtccgccggacagcggctgggcgtccacctgcacaccgaagccagagcccgccatgcctggcagctgactcagggcgctaccgtgctgggcctgttccgcgtgaccccagagatccctgccggcctgccctcccctcggtctgagt

SEQ ID NO: 87-88 - skipped (2 entries)

SEQ ID NO: 89 - protein, 379 aa, Artificial sequence
  379 aa TSP111_V1 “hole”  chain
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 90 - DNA, 1137 bp, Artificial sequence
  na of SEQ ID NO 89
gactctccagataggccttggaatccccctacctttagccccgccctgctggtggtgacagagggcgataacgccaccttcacatgctcttttagcaacacctccgagtctttcgtgctgaattggtacaggatgagcccttccaaccagacagacaagctggcagcatttcctgaggaccgcagccagccaggacaggattcccggttcagagtgacccagctgccaaatggcagggactttcacatgtctgtggtgcgcgcccggagaaacgatagcggcacatacctgtgcggagcaatctccctggcaccaaaggcacagatcaaggagtccctgagggcagagctgagggtgaccgagaggagggccgaggtgccaacagcacacccatctcctagcccaaggccagcaggacagggaggaggaggctctggaggaggaggatccgagtctaagtacggaccaccatgccctccatgtcctgcaccagagttcgagggaggaccatccgtgttcctgtttccacctaagcctaaggacaccctgatgatctccagaacccccgaggtgacatgcgtggtggtggacgtgtctcaggaggatcctgaggtgcagttcaattggtacgtggatggcgtggaggtgcacaacgccaagacaaagccccgggaggagcagtttaattctacctacagagtggtgagcgtgctgacagtgctgcaccaggattggctgaatggcaaggagtataagtgtaaggtgagcaacaagggcctgcctagctccatcgagaagaccatctccaaggccaagggccagccaagagagccccaggtgtacaccctgccacccagccagtgcgagatgacaaagaatcaggtgagcctgtcctgtgccgtgaagggcttctaccctagcgacatcgcagtggagtgggagtccaacggacagccagagaacaattataagaccacacctccagtgctggactccgatggctctttctttctggtgtcccggctgaccgtggataagagccggtggcaggagggcaacgtgttcagctgcagcgtgatgcacgaggccctgcacaaccactatacacagaagtccctgtctctgagcctgggcaag

SEQ ID NO: 91 - protein, 1192 aa, Artificial sequence
  1192 aa TSP111_V1 “knob”  chain
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 92 - DNA, 3576 bp, Artificial sequence
  na of SEQ ID NO 91
gaggaggagctgcaggtcatccagcccgataagtctgtgctggtggcagcaggagagaccgccacactgagatgcaccgccacaagcctgatcccagtgggaccaatccagtggtttaggggagcaggacctggaagggagctgatctacaaccagaaggagggccacttcccaagggtgaccacagtgtccgacctgaccaagcggaacaatatggatttttctatcagaatcggcaatatcacacctgccgacgccggcacctactattgcgtgaagttcagaaagggcagcccagacgatgtggagtttaagtccggagcaggaaccgagctgtctgtgagagcaaagcctagcgccccagtggtgtccggaccagcagcaagggcaaccccacagcacacagtgtccttcacctgtgagtcccacggcttttctccacgcgatatcacactgaagtggttcaagaacggcaatgagctgagcgactttcagaccaacgtggatcccgtgggcgagtctgtgagctactccatccactctacagccaaggtggtgctgacccgggaggacgtgcacagccaggtcatctgcgaggtggcacacgtgaccctgcagggcgatcctctgagaggcacagccaatctgtccgagaccatcagggtgccccctacactggaggtgacccagcagcccgtgagggcagagaaccaagtgaatgtgacatgtcaggtgcggaagttctaccctcagagactgcagctgacctggctggagaacggcaatgtgagccgcaccgagacagcctccaccgtgacagagaacaaggacggcacatataattggatgagctggctgctggtgaacgtgtccgcccacagggacgatgtgaagctgacctgccaggtggagcacgacggacagccagccgtgtctaagagccacgatctgaaggtgtccgcccaccctaaggagcagggctctaacacagccgccgagaataccggcagcaacgagagaaatatctacggaggaggaggatccggaggaggaggatccgagtctaagtatggaccaccatgccctccatgtccagcacctgagtttgagggaggacctagcgtgttcctgtttccccctaagccaaaggacacactgatgatctccaggacaccagaggtgacctgcgtggtggtggacgtgtctcaggaggatcccgaggtgcagttcaactggtacgtggatggcgtggaggtgcacaatgccaagaccaagcctagggaggagcagtttaactctacataccgcgtggtgagcgtgctgaccgtgctgcaccaggattggctgaacggcaaggagtataagtgtaaggtgagcaataagggcctgccaagctccatcgagaagaccatctccaaggcaaagggacagccaagggagcctcaggtgtgcacactgccaccctctcaggaggagatgaccaagaaccaggtgagcctgtggtgtctggtgaagggcttctacccaagcgacatcgccgtggagtgggagtccaatggccagcccgagaacaattacaagaccacacctccagtgctggactctgatggcagcttctttctgtattctaggctgacagtggataagagccgctggcaggagggcaacgtgtttagctgttccgtgatgcacgaggccctgcacaatcactatacccagaagtctctgagcctgtccctgggcaagggcggcggcggcagcggcggaggaggatccgccgcctctcctaggctgagggagggaccagagctgagcccagacgatccagcaggactgctggacctgaggcagggaatgttcgcacagctggtggcccagaacgtgctgctgatcgacggccctctgagctggtactccgatccaggactggcaggcgtgtccctgacaggcggactgtcttataaggaggataccaaggagctggtggtggcaaaggcaggcgtgtactacgtgttcttccagctggagctgaggagagtggtggcaggagagggctctggaagcgtgtccctggccctgcacctgcagccactgcggagcgccgcaggagcagccgccctggccctgaccgtggacctgccacctgcatctagcgaggcacggaattctgccttcggctttcagggcagactgctgcacctgagcgccggacagaggctgggagtgcacctgcacacagaggcaagggcaagacacgcatggcagctgacacagggagcaaccgtgctgggactgttccgcgtgacccctgagatcccagcaggactgccatctccacggagcgagggcggcggcggctctggaggaggaggaagcggaggcggcggctccgccgcttctcccaggctgcgcgagggacctgagctgtccccagacgatcctgccggcctgctggacctgagacagggaatgtttgcccagctggtcgctcagaacgtgctgctgattgacggccccctgtcctggtattccgatcctggactggcaggcgtgagcctgacaggcggcctgagctacaaagaagacactaaagaactggtcgtcgccaaagccggcgtgtactacgtgttcttccaactggagctgaggagggtcgtcgccggcgaaggatccggcagcgtgtccctggccctgcacctgcaaccactgaggagcgccgccggcgccgccgccctggccctgactgtggacctgccaccagcatcctctgaggcaaggaattccgccttcggcttccagggccggctgctgcacctgtctgccggacagagactgggcgtccacctgcataccgaagccagagccaggcatgcctggcagctgacccagggcgccaccgtgctgggcctgttcagagtgaccccagaaattccagcaggactgccttctccaaggagcgagggcggcgggggctccggcggaggaggctctggcggcggcggctccgccgcctctccaaggctgcgcgagggaccagagctgtcccctgatgaccctgccgggctgctggacctgagacaaggcatgttcgcccagctggtcgcacaaaacgtgctgttaattgacggcccactgtcctggtatagcgaccctggcctggccggcgtgtctctgacaggcggactgagctacaaagaagatactaaagaactggtcgtggccaaagctggcgtgtactacgtgttcttccaattagaactgagaagggtcgtcgccggcgagggatccgggagcgtgtccctggccctgcacctgcaaccgctgcggagcgccgccggcgctgccgccctggccctgacagtggacctgcctccagcaagctccgaggcaaggaatagcgccttcggctttcaaggccgcctgctgcacctgtccgccggacagcggctgggcgtccacctgcacaccgaagccagagcccgccatgcctggcagctgactcagggcgctaccgtgctgggcctgttccgcgtgaccccagagatccctgcaggactgccctctcctcggagcgag

SEQ ID NO: 93-94 - skipped (2 entries)

SEQ ID NO: 95 - protein, 21 aa, Artificial sequence
  artificial signal peptide
MESPAQLLFLLLLWLPDGVHA

SEQ ID NO: 96 - protein, 14 aa, Artificial sequence
  linker peptide sequence
GGGGSGGGGSGGGG

SEQ ID NO: 97 - protein, 9 aa, Artificial sequence
  linker peptide sequence
GGGGSGGGG

SEQ ID NO: 98 - protein, 5 aa, Artificial sequence
  linker peptide sequence
  REPEAT (1)..(5): may repeat 1-4 times
GGGGS

SEQ ID NO: 99 - protein, 5 aa, Artificial sequence
  linker peptide sequence
GGGGS

SEQ ID NO: 100 - protein, 10 aa, Artificial sequence
  linker peptide sequence
GGGGSGGGGS

SEQ ID NO: 101 - protein, 5 aa, Artificial sequence
  linker peptide sequence
  REPEAT (1)..(5): may repeat 1-3 times
EAAAK

SEQ ID NO: 102 - protein, 7 aa, Artificial sequence
  linker peptide sequence
  REPEAT (2)..(6): repeat 2-5 times
AEAAAKA

SEQ ID NO: 103 - protein, 12 aa, Artificial sequence
  linker peptide sequence
AEAAAKEAAAKA

SEQ ID NO: 104 - protein, 46 aa, Artificial sequence
  linker peptide sequence
AEAAAKEAAAKEAAAKEAAAKALEAEAAAKEAAAKEAAAKEAAAKA

SEQ ID NO: 105 - protein, 5 aa, Artificial sequence
  linker peptide sequence
PAPAP

SEQ ID NO: 106 - protein, 18 aa, Artificial sequence
  linker peptide sequence
KESGSVSSEQLAQFRSLD

SEQ ID NO: 107 - protein, 14 aa, Artificial sequence
  linker peptide sequence
EGKSSGSGSESKST

SEQ ID NO: 108 - protein, 12 aa, Artificial sequence
  linker peptide sequence
GSAGSAAGSGEF

SEQ ID NO: 109 - protein, 229 aa, Artificial sequence
  linker peptide sequence
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 229 aa, Artificial sequence
  linker peptide sequence
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 229 aa, Artificial sequence
  linker peptide sequence
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 112 - protein, 232 aa, Artificial sequence
  linker peptide sequence
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 232 aa, Artificial sequence
  linker peptide sequence
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 232 aa, Artificial sequence
  linker peptide sequence
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 115 - protein, 398 aa, Artificial sequence
  LILRB2 D1-4, AA 22-419 (Q8N423-1 UniParc) AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 116 - DNA, 1194 bp, Artificial sequence
  LILRB2 D1-4, AA 22-419 (Q8N423-1 UniParc) NA SEQUENCE
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcctggcgtgagcaagaagccatctctgagcgtgcagccaggccctgtggtggcacctggcgagtctctgaccctgcagtgcgtgagcgacgtgggctacgatcggttcgtgctgtataaggagggagagagggatctgaggcagctgccaggcagacagcctcaggcaggactgtcccaggcaaactttacactgggccccgtgagccggagctacggcggacagtaccgctgctatggagcacacaatctgtcctctgagtgttctgcccccagcgaccccctggacatcctgatcaccggccagatcaggggcacaccattcatcagcgtgcagccaggaccaaccgtggcctccggcgagaacgtgacactgctgtgccagagctggcgccagttccacacctttctgctgacaaaggcaggagcagcagacgcacctctgaggctgcgctccatccacgagtacccaaagtatcaggccgagtttccaatgagccccgtgacctccgcccacgcaggcacatacagatgctatggcagcctgaacagcgacccctacctgctgagccacccttccgagccactggagctggtggtgtcc

SEQ ID NO: 117 - protein, 198 aa, Artificial sequence
  LILRB2 D1-2, AA 22-219 (Q8N423-1 UniParc) AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVP

SEQ ID NO: 118 - DNA, 594 bp, Artificial sequence
  LILRB2 D1-2, AA 22-219 (Q8N423-1 UniParc) NA SEQUENCE
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcct

SEQ ID NO: 119 - protein, 148 aa, Artificial sequence
  CD40L, AA 114-261 C194S (P29965 Uniprot) AA SEQUENCE
QKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 120 - DNA, 444 bp, Artificial sequence
  CD40L, AA 114-261 C194S (P29965 Uniprot) NA SEQUENCE
cagaagggcgatcagaatcctcagattgccgcccacgtgatctctgaggcctctagcaagaccacaagcgtgctgcagtgggcagaaaaagggtactatacaatgtctaacaatctggtgaccctggagaacggcaagcaactgaccgtcaaaagacagggcctgtactacatctacgctcaggtgaccttctgcagcaatagggaggcctcctctcaggcaccttttatcgcctctctgagcctgaagtccccaggccggttcgagagaatcctgttaagggcagcaaacactcacagctccgccaaaccatgtggacagcagagcatacacctgggcggcgtgttcgagctgcaaccgggagcctccgtgtttgtcaatgtgaccgacccatcccaggtgtctcacggcaccggcttcaccagcttcggcctgctgaagctg

SEQ ID NO: 121 - protein, 474 aa, Artificial sequence
  sc3xCD40L, AA 114-261 C194S (P29965 Uniprot) internal linker 3x(GGGGS) AA SEQUENCE
QKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 122 - protein, 215 aa, Artificial sequence
  CD40L full ECD, AA 47-261 (P29965 Uniprot) AA SEQUENCE
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 123 - protein, 148 aa, Artificial sequence
  CD40L, AA 114-261 NO MUTATION (P29965 Uniprot) AA SEQUENCE
QKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 124 - DNA, 444 bp, Artificial sequence
  CD40L, AA 114-261 NO MUTATION (P29965 Uniprot) NA SEQUENCE
cagaagggcgatcagaatcctcagattgccgcccacgtgatctctgaggcctctagcaagaccacaagcgtgctgcagtgggcagaaaaagggtactatacaatgtctaacaatctggtgaccctggagaacggcaagcaactgaccgtcaaaagacagggcctgtactacatctacgctcaggtgaccttctgcagcaatagggaggcctcctctcaggcaccttttatcgcctctctgtgcctgaagtccccaggccggttcgagagaatcctgttaagggcagcaaacactcacagctccgccaaaccatgtggacagcagagcatacacctgggcggcgtgttcgagctgcaaccgggagcctccgtgtttgtcaatgtgaccgacccatcccaggtgtctcacggcaccggcttcaccagcttcggcctgctgaagctg

SEQ ID NO: 125 - protein, 128 aa, Artificial sequence
  Siglec 10, AA 18-145 NO MUTATION (Q96LC7-1 Uniprot) AA SEQUENCE
DGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQK

SEQ ID NO: 126 - DNA, 186 bp, Artificial sequence
  Siglec 10, AA 18-145 NO MUTATION (Q96LC7-1 Uniprot) AA SEQUENCE
gatggccggttttggatcagagtgcaggagtccgtgatggtgcctgagggcctgtgcatcagcgtgccatgctccttctcttaccccagacaggactggaccggctctacacccgcctacggctattggtttaaggccgtgaccgagacaacaaagggcgcccctgtggccacaaaccaccagagc

SEQ ID NO: 127 - protein, 128 aa, Artificial sequence
  Siglec 10, AA 18-145 plus C36S (Q96LC7-1 Uniprot) AA SEQUENCE
DGRFWIRVQESVMVPEGLSISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQK

SEQ ID NO: 128 - DNA, 384 bp, Artificial sequence
  Siglec 10, AA 18-145 plus C36S (Q96LC7-1 Uniprot) NA SEQUENCE
gatggccggttttggatcagagtgcaggagtccgtgatggtgcctgagggcctgtctatcagcgtgccatgctccttctcttaccccagacaggactggaccggctctacacccgcctacggctattggtttaaggccgtgaccgagacaacaaagggcgcccctgtggccacaaaccaccagagcagagaggtggagatgtccacccggggcagattccagctgacaggcgaccccgccaagggcaattgtagcctggtcatcagggacgcccagatgcaggatgagtctcagtacttctttagggtggagcgcggcagctacgtgcgctataactttatgaatgatggcttctttctgaaggtgaccgccctgacacagaag

SEQ ID NO: 129 - protein, 534 aa, Artificial sequence
  Siglec 10 full ECD, AA 17-550 (Q96LC7-1 Uniprot) AA SEQUENCE
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSN

SEQ ID NO: 130 - protein, 112 aa, Artificial sequence
  TIGIT, AA 22-134 NO MUTATIONS (Q495A1 Uniprot ID) AA SEQUENCE
MTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEH

SEQ ID NO: 131 - DNA, 336 bp, Artificial sequence
  TIGIT, ECD AA 22-141 NO MUTATIONS (Q495A1 Uniprot ID) NA SEQUENCE
atgaccggcacaatcgagacaacaggcaacatctctgccgagaagggaggcagcatcatcctgcagtgccacctgagcagcaccacagcccaggtgacccaggtgaactgggagcagcaggaccagctgctggccatctgcaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcac

SEQ ID NO: 132 - protein, 112 aa, Artificial sequence
  TIGIT, AA 22-134 plus I42A C69S (Q495A1 Uniprot ID) AA SEQUENCE
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEH

SEQ ID NO: 133 - DNA, 336 bp, Artificial sequence
  TIGIT, AA 22-134 plus I42A C69S (Q495A1 Uniprot ID) NA SEQUENCE
atgaccggcacaatcgagacaacaggcaacatctctgccgagaagggaggcagcatcgccctgcagtgccacctgagcagcaccacagcccaggtgacccaggtgaactgggagcagcaggaccagctgctggccatctccaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcac

SEQ ID NO: 134 - protein, 15 aa, Artificial sequence
  LINKER amino acid sequence
GGGGSGGGGSGGGGS

SEQ ID NO: 135 - protein, 25 aa, Artificial sequence
  LINKER amino acid sequence
GGGGSGGGGSGGGGSGGGGSGGGGS

SEQ ID NO: 136 - protein, 8 aa, Artificial sequence
  LINKER amino acid sequence
GGGGGGGG

SEQ ID NO: 137 - protein, 8 aa, Artificial sequence
  LINKER amino acid sequence
GGGGGGGG

SEQ ID NO: 138 - protein, 642 aa, Artificial sequence
  LILRB2 D1-4 Fc IgG4 Knob: in TSP 214 and 215 217  218 221 and 222 AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - DNA, 1926 bp, Artificial sequence
  LILRB2 D1-4 Fc IgG4 Knob: in TSP 214 and 215 217  218 221 and 222 NA SEQUENCE
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcctggcgtgagcaagaagccatctctgagcgtgcagccaggccctgtggtggcacctggcgagtctctgaccctgcagtgcgtgagcgacgtgggctacgatcggttcgtgctgtataaggagggagagagggatctgaggcagctgccaggcagacagcctcaggcaggactgtcccaggcaaactttacactgggccccgtgagccggagctacggcggacagtaccgctgctatggagcacacaatctgtcctctgagtgttctgcccccagcgaccccctggacatcctgatcaccggccagatcaggggcacaccattcatcagcgtgcagccaggaccaaccgtggcctccggcgagaacgtgacactgctgtgccagagctggcgccagttccacacctttctgctgacaaaggcaggagcagcagacgcacctctgaggctgcgctccatccacgagtacccaaagtatcaggccgagtttccaatgagccccgtgacctccgcccacgcaggcacatacagatgctatggcagcctgaacagcgacccctacctgctgagccacccttccgagccactggagctggtggtgtccggcggcggcggctctggcggaggaggcagcggaggaggaggatccgagtctaagtacggaccaccatgccctccatgtcctgcaccagagttcgagggaggaccatccgtgttcctgtttccacctaagcctaaggacaccctgatgatctccagaacccccgaggtgacatgcgtggtggtggacgtgtctcaggaggatcctgaggtgcagttcaattggtacgtggatggcgtggaggtgcacaacgccaagacaaagccccgggaggagcagtttaatagcacctacagagtggtgtccgtgctgacagtgctgcaccaggattggctgaatggcaaggagtataagtgtaaggtgagcaacaagggcctgcctagctccatcgagaagaccatctccaaggccaagggccagccaagagagccacaggtgtgcaccctgccaccaagccaggaggagatgacaaagaatcaggtgtccctgtggtgtctggtgaagggcttctacccttccgacatcgccgtggagtgggagtctaacggccagccagagaacaattacaagaccacacctccagtgctggactctgatggcagcttctttctgtattctcggctgaccgtggataagagcagatggcaggagggcaacgtgttcagctgctccgtgatgcacgaggccctgcacaaccactatacacagaagtctctgagcctgtccctgggcaag

SEQ ID NO: 140 - protein, 442 aa, Artificial sequence
  LILRB2 D1-2 Fc IgG4 Knob: in TSP 214 V1 and 215 V1 AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - DNA, 1326 bp, Artificial sequence
  LILRB2 D1-2 Fc IgG4 Knob: in TSP 214 V1 and 215 V1 NA SEQUENCE
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcctggcggcggcggctctggcggaggaggcagcggaggaggaggatccgagtctaagtacggaccaccatgccctccatgtcctgcaccagagttcgagggaggaccatccgtgttcctgtttccacctaagcctaaggacaccctgatgatctccagaacccccgaggtgacatgcgtggtggtggacgtgtctcaggaggatcctgaggtgcagttcaattggtacgtggatggcgtggaggtgcacaacgccaagacaaagccccgggaggagcagtttaatagcacctacagagtggtgtccgtgctgacagtgctgcaccaggattggctgaatggcaaggagtataagtgtaaggtgagcaacaagggcctgcctagctccatcgagaagaccatctccaaggccaagggccagccaagagagccacaggtgtgcaccctgccaccaagccaggaggagatgacaaagaatcaggtgtccctgtggtgtctggtgaagggcttctacccttccgacatcgccgtggagtgggagtctaacggccagccagagaacaattacaagaccacacctccagtgctggactctgatggcagcttctttctgtattctcggctgaccgtggataagagcagatggcaggagggcaacgtgttcagctgctccgtgatgcacgaggccctgcacaaccactatacacagaagtctctgagcctgtccctgggcaag

SEQ ID NO: 142 - protein, 1252 aa, Artificial sequence
  LILRB2 D1-4 -Fc IgG4 Hole-sc3x41BBL: in DSP 214 AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 143 - DNA, 3756 bp, Artificial sequence
  LILRB2 D1-4 -Fc IgG4 Hole-sc3x41BBL: in DSP 214 AA SEQUENCE NA SEQUENCE
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcctggcgtgagcaagaagccatctctgagcgtgcagccaggccctgtggtggcacctggcgagtctctgaccctgcagtgcgtgagcgacgtgggctacgatcggttcgtgctgtataaggagggagagagggatctgaggcagctgccaggcagacagcctcaggcaggactgtcccaggcaaactttacactgggccccgtgagccggagctacggcggacagtaccgctgctatggagcacacaatctgtcctctgagtgttctgcccccagcgaccccctggacatcctgatcaccggccagatcaggggcacaccattcatcagcgtgcagccaggaccaaccgtggcctccggcgagaacgtgacactgctgtgccagagctggcgccagttccacacctttctgctgacaaaggcaggagcagcagacgcacctctgaggctgcgctccatccacgagtacccaaagtatcaggccgagtttccaatgagccccgtgacctccgcccacgcaggcacatacagatgctatggcagcctgaacagcgacccctacctgctgagccacccttccgagccactggagctggtggtgtccggcggcggcggctctggaggaggaggatccggaggaggaggatccgagtctaagtatggaccaccatgccctccatgtccagcacctgagtttgagggaggaccttccgtgttcctgtttccccctaagccaaaggacacactgatgatctccaggacaccagaggtgacctgcgtggtggtggacgtgtctcaggaggatcccgaggtgcagttcaactggtacgtggatggcgtggaggtgcacaatgccaagaccaagcctagggaggagcagtttaactccacataccgcgtggtgtctgtgctgaccgtgctgcaccaggattggctgaacggcaaggagtataagtgtaaggtgagcaataagggcctgccaagctccatcgagaagaccatctccaaggcaaagggacagccaagggagcctcaggtgtatacactgccacccagccagtgcgagatgaccaagaaccaggtgagcctgtcctgtgccgtgaagggcttctacccatctgacatcgccgtggagtgggagagcaatggccagcccgagaacaattataagaccacacctccagtgctggactccgatggctctttctttctggtgtccaggctgacagtggataagtctcgctggcaggagggcaacgtgttttcttgtagcgtgatgcacgaggccctgcacaatcactacacccagaagtccctgtctctgagcctgggcaagggcggcggcggctccggaggaggaggaagcgccgcctcccctaggctgcgcgagggaccagagctgagcccagacgatccagcaggactgctggacctgaggcagggaatgttcgcacagctggtggcccagaacgtgctgctgatcgacggccctctgtcttggtacagcgatccaggactggcaggcgtgagcctgacaggcggactgtcctataaggaggataccaaggagctggtggtggcaaaggcaggcgtgtactacgtgttcttccagctggagctgaggagagtggtggcaggagagggatccggatctgtgagcctggccctgcacctgcagccactgcggagcgccgcaggagcagccgccctggccctgaccgtggacctgccacctgcatctagcgaggcacggaattctgccttcggctttcagggcagactgctgcacctgagcgccggacagaggctgggagtgcacctgcacacagaggcaagggcaagacacgcatggcagctgacacagggagcaaccgtgctgggactgttccgcgtgacccctgagatcccagcaggactgccatccccccggtctgagggcggcggcgggtccggaggaggaggatctggcggcggcggcagcgccgcctcccccaggctgcgcgagggacctgagctgtccccagacgatcctgccggcctgctggacctgagacagggaatgtttgcccagctggtcgctcagaacgtgctgctgattgacggccccctgtcctggtatagcgatcctggactggcaggcgtgtctctgacaggcggactgagttacaaagaagacactaaagaactggtcgtcgccaaagccggcgtgtactacgtgttcttccaactggagctgaggagggtcgtcgccggcgaaggcagcggctctgtgagcctggccctgcacctgcaaccactgaggagcgccgccggcgccgccgccctggccctgactgtggacctgccaccagcatcctctgaggcaaggaattccgccttcggcttccagggccggctgctgcacctgtctgccggacagagactgggcgtccacctgcataccgaagccagagccaggcatgcctggcagctgacccagggcgccaccgtgctgggcctgttcagagtgaccccagaaattccagcaggactgccttccccaaggtctgagggaggaggaggaagtggcggaggaggatccggagggggagggagcgccgcctccccaagactgagagagggaccagagctgtcccctgatgaccctgccgggctgctggacctgagacaaggcatgttcgcccagctggtcgcacaaaacgtgctgttaattgacggcccactgtcctggtattccgaccctggcctggccggcgtgtccctgacaggcggcctgtcttacaaagaagacacaaaagaactggtcgtcgctaaagctggcgtgtactacgtgttcttccaattagaactgagaagggtcgtcgccggcgagggcagcgggtctgtgagcctggccctgcacctgcaaccgctgcggagcgccgccggcgctgccgccctggccctgacagtggacctgcctccagcaagctccgaggcaaggaatagcgccttcggctttcaaggccgcctgctgcacctgtccgccggacagcggctgggcgtccacctgcacaccgaagccagagcccgccatgcctggcagctgactcagggcgctaccgtgctgggcctgttccgcgtgaccccagagatccctgccggcctgccctcccctcggtctgag

SEQ ID NO: 144 - protein, 1052 aa, Artificial sequence
  LILRB2 D1-2- Fc IgG4 Hole- sc3x41BBL: in DSP 214 V1 AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 145 - DNA, 3156 bp, Artificial sequence
  LILRB2 D1-2- Fc IgG4 Hole- sc3x41BBL: in DSP 214 V1 NA SEQUENCE
cagaccggcacaatccccaagcctaccctgtgggccgagccagatagcgtgatcacccagggctcccccgtgacactgtcttgccagggcagcctggaggcacaggagtaccggctgtatagagagaagaagagcgcctcctggatcacccggatcagacccgagctggtgaagaacggccagtttcacatcccttccatcacctgggagcacacaggccggtacggatgccagtactattctcgggccagatggagcgagctgtccgaccccctggtgctggtcatgaccggcgcctatccaaagcccacactgtccgcccagccttctccagtggtgacctctggcggcagagtgacactgcagtgtgagagccaggtggccttcggcggctttatcctgtgcaaggagggcgaggaggagcacccccagtgtctgaatagccagcctcacgcccggggcagctccagagccatcttctctgtgggccctgtgagcccaaaccggagatggtcccacaggtgctacggctatgacctgaacagcccatacgtgtggtctagcccctctgatctgctggagctgctggtgcctggcggcggcggctctggaggaggaggatccggaggaggaggatccgagtctaagtatggaccaccatgccctccatgtccagcacctgagtttgagggaggaccttccgtgttcctgtttccccctaagccaaaggacacactgatgatctccaggacaccagaggtgacctgcgtggtggtggacgtgtctcaggaggatcccgaggtgcagttcaactggtacgtggatggcgtggaggtgcacaatgccaagaccaagcctagggaggagcagtttaactccacataccgcgtggtgtctgtgctgaccgtgctgcaccaggattggctgaacggcaaggagtataagtgtaaggtgagcaataagggcctgccaagctccatcgagaagaccatctccaaggcaaagggacagccaagggagcctcaggtgtatacactgccacccagccagtgcgagatgaccaagaaccaggtgagcctgtcctgtgccgtgaagggcttctacccatctgacatcgccgtggagtgggagagcaatggccagcccgagaacaattataagaccacacctccagtgctggactccgatggctctttctttctggtgtccaggctgacagtggataagtctcgctggcaggagggcaacgtgttttcttgtagcgtgatgcacgaggccctgcacaatcactacacccagaagtccctgtctctgagcctgggcaagggcggcggcggctccggaggaggaggaagcgccgcctcccctaggctgcgcgagggaccagagctgagcccagacgatccagcaggactgctggacctgaggcagggaatgttcgcacagctggtggcccagaacgtgctgctgatcgacggccctctgtcttggtacagcgatccaggactggcaggcgtgagcctgacaggcggactgtcctataaggaggataccaaggagctggtggtggcaaaggcaggcgtgtactacgtgttcttccagctggagctgaggagagtggtggcaggagagggatccggatctgtgagcctggccctgcacctgcagccactgcggagcgccgcaggagcagccgccctggccctgaccgtggacctgccacctgcatctagcgaggcacggaattctgccttcggctttcagggcagactgctgcacctgagcgccggacagaggctgggagtgcacctgcacacagaggcaagggcaagacacgcatggcagctgacacagggagcaaccgtgctgggactgttccgcgtgacccctgagatcccagcaggactgccatccccccggtctgagggcggcggcgggtccggaggaggaggatctggcggcggcggcagcgccgcctcccccaggctgcgcgagggacctgagctgtccccagacgatcctgccggcctgctggacctgagacagggaatgtttgcccagctggtcgctcagaacgtgctgctgattgacggccccctgtcctggtatagcgatcctggactggcaggcgtgtctctgacaggcggactgagttacaaagaagacactaaagaactggtcgtcgccaaagccggcgtgtactacgtgttcttccaactggagctgaggagggtcgtcgccggcgaaggcagcggctctgtgagcctggccctgcacctgcaaccactgaggagcgccgccggcgccgccgccctggccctgactgtggacctgccaccagcatcctctgaggcaaggaattccgccttcggcttccagggccggctgctgcacctgtctgccggacagagactgggcgtccacctgcataccgaagccagagccaggcatgcctggcagctgacccagggcgccaccgtgctgggcctgttcagagtgaccccagaaattccagcaggactgccttccccaaggtctgagggaggaggaggaagtggcggaggaggatccggagggggagggagcgccgcctccccaagactgagagagggaccagagctgtcccctgatgaccctgccgggctgctggacctgagacaaggcatgttcgcccagctggtcgcacaaaacgtgctgttaattgacggcccactgtcctggtattccgaccctggcctggccggcgtgtccctgacaggcggcctgtcttacaaagaagacacaaaagaactggtcgtcgctaaagctggcgtgtactacgtgttcttccaattagaactgagaagggtcgtcgccggcgagggcagcgggtctgtgagcctggccctgcacctgcaaccgctgcggagcgccgccggcgctgccgccctggccctgacagtggacctgcctccagcaagctccgaggcaaggaatagcgccttcggctttcaaggccgcctgctgcacctgtccgccggacagcggctgggcgtccacctgcacaccgaagccagagcccgccatgcctggcagctgactcagggcgctaccgtgctgggcctgttccgcgtgaccccagagatccctgccggcctgccctcccctcggtctgag

SEQ ID NO: 146 - protein, 1076 aa, Artificial sequence
  SIRPa -Fc Hole IgG4- sc3xCD40L: in TSP 112 and 217 AA SEQUENCE
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 147 - DNA, 3228 bp, Artificial sequence
  SIRPa -Fc Hole IgG4- sc3xCD40L: in TSP 112 and 217 NA SEQUENCE
gaggaggagctgcaggtcatccagcctgataagtctgtgctggtggcagcaggagagacagccacactgcggtgcaccgccacaagcctgatcccagtgggccccatccagtggttcaggggagcaggacctggaagggagctgatctacaaccagaaggagggccactttccaagagtgaccacagtgtccgacctgaccaagcggaacaatatggatttctctatcagaatcggcaatatcacacctgccgacgccggcacctactattgcgtgaagttcaggaagggctccccagacgatgtggagtttaagagcggagcaggcaccgagctgtccgtgagggccaagccttctgccccagtggtgagcggaccagcagccagagccaccccacagcacacagtgagcttcacctgtgagagccacggcttttccccccgggacatcaccctgaagtggttcaagaacggcaatgagctgtccgactttcagaccaacgtggaccccgtgggcgagtctgtgagctattccatccactctacagccaaggtggtgctgacccgcgaggacgtgcacagccaggtcatctgcgaggtggcacacgtgaccctgcagggcgatcccctgaggggcacagccaatctgagcgagacaatcagagtgccccctacactggaggtgacccagcagcccgtgcgcgcagagaaccaagtgaatgtgacatgtcaggtgcggaagttctaccctcagagactgcagctgacctggctggagaacggcaacgtgagccggaccgagacagccagcaccgtgacagagaacaaggacggcacatataattggatgtcttggctgctggtgaacgtgagcgcccacagggacgatgtgaagctgacctgccaggtggagcacgacggacagccagccgtgtctaagagccacgatctgaaggtgagcgcccaccctaaggagcagggctccaacacagccgccgagaataccggcagcaacgagcggaatatctacggaggaggaggctccggaggaggaggctccgagtctaagtatggaccaccatgccctccatgtccagcacctgagttcgagggaggacctagcgtgttcctgtttccccctaagccaaaggacaccctgatgatctccagaacaccagaggtgacctgcgtggtggtggacgtgtctcaggaggaccccgaggtgcagtttaactggtacgtggatggcgtggaggtgcacaatgccaagaccaagccccgggaggagcagttcaattccacataccgcgtggtgtctgtgctgaccgtgctgcaccaggactggctgaacggcaaggagtataagtgtaaggtgagcaataagggcctgccaagctccatcgagaaaaccatcagcaaggcaaagggacagccccgggagcctcaggtgtatacactgccaccctcccagtgcgagatgaccaagaaccaggtgagcctgtcctgtgccgtgaagggcttttacccatctgacatcgccgtggagtgggagagcaatggccagcccgagaacaattataagaccacacctccagtgctggactccgatggctctttctttctggtgtccaggctgacagtggataagtctcgctggcaggagggcaacgtgttctcttgtagcgtgatgcacgaggccctgcacaatcactacacccaaaagtccctgtctctgagcctgggcaagggcggcggcggctccggcggaggaggctctggcggcggcggcagcggaggcggcggctcccagaagggcgatcagaacccccagatcgccgcccacgtgatctccgaggcctctagcaagaccacatctgtgctgcagtgggccgagaagggctactatacaatgagcaacaatctggtgaccctggagaacggcaagcagctgacagtgaagcggcagggcctgtactatatctatgcccaggtgaccttctgctccaatagagaggcctcctctcaggccccctttatcgcctccctgtctctgaagtctcctggccggttcgagagaatcctgctgagggcagcaaacacacacagctccgccaagccctgtggccagcagtctatccacctgggcggcgtgttcgagctgcagccaggagccagcgtgtttgtgaatgtgaccgaccctagccaggtgtcccacggcaccggcttcaccagcttcggcctgctgaagctgggcggcggcggctctggcggaggaggcagcggaggaggaggctcccagaaaggcgatcagaaccctcagattgctgcccacgtgatcagcgaggcctctagcaagaccacatccgtgctgcagtgggctgaaaaaggatactatacaatgtctaataatctggtgaccctggagaatggcaagcagctgaccgtcaagagacagggcctgtattatatctacgcccaggtgaccttttgcagcaatcgcgaggcctcctctcaggctccattcatcgccagcctgtccctgaagtccccaggcaggtttgagcgcatcctgctgagagccgccaatacccacagctccgccaagccatgtggacagcagtccattcacctgggcggcgtgttcgagctgcaaccaggagccagcgtgttcgtgaatgtgaccgacccctctcaggtgagccacggcaccggcttcaccagcttcggcctgctgaagctgggtggaggaggctctggcgggggcggcagcggcggaggaggctcccagaagggcgatcagaatcctcagattgccgcccacgtgatctctgaggcctctagcaagaccacaagcgtgctgcagtgggcagaaaaagggtactatacaatgtctaacaatctggtgaccctggagaacggcaagcaactgaccgtcaaaagacagggcctgtactacatctacgctcaggtgaccttctgcagcaatagggaggcctcctctcaggcaccttttatcgcctctctgagcctgaagtccccaggccggttcgagagaatcctgttaagggcagcaaacactcacagctccgccaaaccatgtggacagcagagcatacacctgggcggcgtgttcgagctgcaaccgggagcctccgtgtttgtcaatgtgaccgacccatcccaggtgtctcacggcaccggcttcaccagcttcggcctgctgaagctg

SEQ ID NO: 148 - protein, 1136 aa, Artificial sequence
  LILRB2-Fc -Fc Hole IgG4- sc3xCD40L: in TSP 218 AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 149 - DNA, 3408 bp, Artificial sequence
  LILRB2-Fc -Fc Hole IgG4- sc3xCD40L: in TSP 218 NA SEQUENCE
cagaccggcacaatccctaagccaaccctgtgggccgagcctgatagcgtgatcacccagggctccccagtgacactgagctgccagggctccctggaggcacaggagtaccggctgtatagagagaagaagtctgccagctggatcacccggatcagacctgagctggtgaagaacggccagttccacatcccatctatcacctgggagcacacaggccggtacggatgccagtactatagccgggccagatggtctgagctgagcgaccccctggtgctggtcatgaccggagcctatcccaagcctacactgtctgcccagccaagcccagtggtgacctctggcggcagagtgacactgcagtgtgagagccaggtggccttcggcggctttatcctgtgcaaggagggcgaggaggagcacccacagtgtctgaatagccagccacacgccaggggcagctcccgcgccatcttcagcgtgggacccgtgagcccaaaccggagatggtcccaccgctgctacggctatgacctgaacagcccttacgtgtggtctagcccaagcgatctgctggagctgctggtgcccggcgtgagcaagaagccttccctgtctgtgcagcctggaccagtggtggcacctggagagtccctgaccctgcagtgcgtgagcgacgtgggctacgatcggtttgtgctgtataaggagggagagagggatctgaggcagctgccaggcagacagccacaggccggcctgtcccaggccaacttcaccctgggcccagtgagccggtcctatggcggccagtacagatgctatggcgcccacaatctgtcctctgagtgttccgccccaagcgaccccctggacatcctgatcaccggccagatcaggggcacaccctttatcagcgtgcagccaggacctaccgtggcctccggcgagaacgtgacactgctgtgccagagctggcgccagttccacacctttctgctgacaaaggcaggagcagcagacgcaccactgaggctgcgctccatccacgagtaccccaagtatcaggccgagttcccaatgtccccagtgacctctgcccacgcaggcacatacaggtgttatggcagcctgaacagcgacccctacctgctgtctcaccctagcgagccactggagctggtggtgtctggaggaggaggcagcggcggaggaggctccggaggcggcggctctgagagcaagtatggaccaccttgcccaccatgtccagcaccagagttcgagggaggaccaagcgtgttcctgtttcctccaaagcctaaggacaccctgatgatctcccgcacccctgaggtgacatgcgtggtggtggacgtgtctcaggaggaccccgaggtgcagtttaactggtacgtggatggcgtggaggtgcacaatgccaagaccaagccccgggaggagcagttcaactctacctacagagtggtgagcgtgctgacagtgctgcaccaggactggctgaacggcaaggagtataagtgtaaggtgagcaataagggcctgcctagctccatcgagaaaaccatcagcaaggcaaagggacagcccagggagcctcaggtgtataccctgcccccttcccagtgcgagatgacaaagaaccaggtgtccctgtcttgtgccgtgaagggcttttacccatccgacatcgccgtggagtgggagtctaatggccagcccgagaacaattataagaccacaccacccgtgctggactccgatggctctttctttctggtgagcaggctgaccgtggataagtcccgctggcaggagggcaacgtgttcagctgctccgtgatgcacgaggccctgcacaatcactacacacagaagtctctgagcctgtccctgggcaagggcggcggcggctctggcggcggcggcagcggcggcggcggctccggaggaggcggctctcagaagggcgatcagaacccccagatcgccgcccacgtgatcagcgaggcctctagcaagaccacatccgtgctgcagtgggccgagaagggctactataccatgagcaacaatctggtgacactggagaacggcaagcagctgaccgtgaagaggcagggcctgtactatatctacgcccaggtgacattctgctccaatcgcgaggcctcctctcaggccccttttatcgcctctctgagcctgaagtctccaggccggttcgagagaatcctgctgagggcagcaaacacccacagctccgccaagccttgtggccagcagtctatccacctgggcggcgtgttcgagctgcagccaggagccagcgtgtttgtgaatgtgacagaccctagccaggtgtcccacggcaccggcttcaccagcttcggcctgctgaagctgggcggcggcggcagcgggggcggcggctccggagggggaggctctcagaaaggcgatcagaatccacagattgctgcccacgtgatctctgaggcctctagcaagaccacaagcgtgctgcagtgggctgaaaaaggatactataccatgtctaataatctggtgacactggagaatggcaagcagctgactgtcaagagacagggcctgtattacatctacgctcaggtgacattttgcagcaatagagaggcctcctctcaggctcccttcatcgcctccctgtctctgaagtcccctggcaggtttgagcgcatcctgctgagagccgccaatactcacagctccgccaagccatgtggacagcagtccattcacctgggcggcgtgttcgagctgcaaccaggagccagcgtgttcgtgaatgtgacagacccctctcaggtgagccacggcaccggcttcaccagcttcggcctgctgaagctgggtggcggcggcagcggcgggggcggctccggaggcggaggctctcagaagggcgatcagaatcctcagattgcagcccacgtgatctccgaggcctctagcaagaccacatctgtgctgcagtgggctgagaaaggctactataccatgtctaacaatctggtgacactggagaacggcaagcaactgactgtcaaaagacagggcctgtattatatctacgctcaagtgacattctgcagcaatcgggaggcctcctctcaggcacccttcatcgccagcctgtccctgaagtcccctggccggttcgagagaatcctgttaagagccgccaatacacacagctccgccaaaccatgtggacagcagagcatacacctgggcggcgtgttcgagctgcaaccgggagcctccgtgtttgtcaatgtgacagacccatcccaggtgtctcacggcaccggcttcaccagcttcggcctgctgaagctg

SEQ ID NO: 150 - protein, 367 aa, Artificial sequence
  Siglec-Fc Knob IgG4: in TSP 401 and 403 AA SEQUENCE
DGRFWIRVQESVMVPEGLSISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 151 - DNA, 1101 bp, Artificial sequence
  Siglec-Fc Knob IgG4: in TSP 401 and 403 NA SEQUENCE
gatggccggttttggatcagagtgcaggagtccgtgatggtgcctgagggcctgtctatcagcgtgccatgctccttctcttaccccagacaggactggaccggctctacacccgcctacggctattggtttaaggccgtgaccgagacaacaaagggcgcccctgtggccacaaaccaccagagcagagaggtggagatgtccacccggggcagattccagctgacaggcgaccccgccaagggcaattgtagcctggtcatcagggacgcccagatgcaggatgagtctcagtacttctttagggtggagcgcggcagctacgtgcgctataactttatgaatgatggcttctttctgaaggtgaccgccctgacacagaagggaggaggaggctccggcggaggaggcagcgagtccaagtatggaccaccttgcccaccatgtcctgcaccagagttcgagggaggacctagcgtgttcctgtttcctccaaagccaaaggacaccctgatgatcagcaggactcctgaggtgacatgcgtggtggtggacgtgtcccaggaggaccccgaggtgcagttcaactggtatgtggatggcgtggaggtgcacaatgccaagacaaagccacgggaggagcagtttaactctacctacagagtggtgagcgtgctgacagtgctgcaccaggattggctgaacggcaaggagtataagtgtaaggtgtctaataagggcctgcccagctccatcgagaaaaccatcagcaaggcaaagggacagccccgggagcctcaggtgtgcaccctgcccccttcccaggaggagatgacaaagaaccaggtgtctctgtggtgtctggtgaagggcttctacccaagcgacatcgccgtggagtgggagtccaatggccagcccgagaacaattacaagaccacaccacccgtgctggactccgatggctctttctttctgtattccaggctgaccgtggataagtctcgctggcaggagggcaacgtgttttcttgcagcgtgatgcacgaggccctgcacaatcactatacacagaagtccctgtctctgagcctgggcaag

SEQ ID NO: 152 - protein, 351 aa, Artificial sequence
  TIGIT-Fc Knob IgG4: in TSP 501 and 503 AA SEQUENCE
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 153 - DNA, 1053 bp, Artificial sequence
  TIGIT-Fc Knob IgG4: in TSP 501 and 503 NA SEQUENCE
atgaccggcacaatcgagacaacaggcaacatctctgccgagaagggaggcagcatcgccctgcagtgccacctgagcagcaccacagcccaggtgacccaggtgaactgggagcagcaggaccagctgctggccatctccaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcacggaggaggaggctccggaggaggaggctctgagagcaagtacggaccaccttgcccaccatgtccagcacctgagttcgagggaggacctagcgtgttcctgtttcctccaaagccaaaggacaccctgatgatcagcaggacccctgaggtgacatgcgtggtggtggacgtgtcccaggaggaccccgaggtgcagttcaactggtatgtggatggcgtggaggtgcacaatgccaagacaaagcccagggaggagcagtttaactccacctaccgcgtggtgtctgtgctgacagtgctgcaccaggactggctgaacggcaaggagtataagtgtaaggtgtctaataagggcctgccctcctctatcgagaaaaccatcagcaaggccaagggccagccaagagagccacaggtgtgcaccctgccaccttcccaggaggagatgacaaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtatagccggctgaccgtggataagtccagatggcaggagggcaacgtgttttcctgctctgtgatgcacgaggccctgcacaatcactatacacagaagagcctgtccctgtctctgggcaag

SEQ ID NO: 154 - protein, 873 aa, Artificial sequence
  PD1- Fc Hole IgG4-sc3xCD40L: in TSP222 and 403 and 503 AA SEQUENCE
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 155 - DNA, 2619 bp, Artificial sequence
  PD1- Fc Hole IgG4-sc3xCD40L: in TSP222 and 403 and 503 AA SEQUENCE
gacagcccagatagaccttggaatccacctaccttctcccccgccctgctggtggtgacagagggcgataacgccaccttcacatgctcttttagcaacacctccgagtcttttgtgctgaattggtacaggatgagcccttccaaccagacagacaagctggccgcattccctgaggaccgctcccagccaggacaggattctcggttcagagtgacccagctgccaaatggccgggactttcacatgagcgtggtgagagcccggagaaacgattccggcacatacctgtgcggagccatctctctggccccaaaggcacagatcaaggagtccctgcgggcagagctgagagtgaccgagaggagggcagaggtgcccacagcacacccatctcctagcccacggcccgcaggacagggaggaggaggcagcgggggaggaggctccgagtctaagtacggaccaccatgccctccatgtcctgcaccagagttcgagggaggaccatccgtgttcctgtttccacctaagcctaaggacaccctgatgatcagccggaccccagaggtgacatgcgtggtggtggacgtgagccaggaggaccccgaggtgcagtttaattggtatgtggatggcgtggaggtgcacaacgccaagaccaagcccagggaggagcagttcaacagcacctaccgcgtggtgtccgtgctgacagtgctgcaccaggactggctgaatggcaaggagtataagtgtaaggtgtccaacaagggcctgcctagctccatcgagaaaaccatcagcaaggcaaagggacagccacgggagccacaggtgtacaccctgccaccaagccagtgcgagatgacaaagaatcaggtgagcctgtcctgtgccgtgaagggcttttacccttccgacatcgccgtggagtgggagtctaacggccagccagagaacaattataagaccacacctccagtgctggactccgatggctctttctttctggtgtctaggctgaccgtggataagagccgctggcaggagggcaacgtgttcagctgcagcgtgatgcacgaggccctgcacaaccactatacacaaaagtccctgtctctgagcctgggcaagggcggcggcggcagcggcggaggaggctccggaggcggcggctctggcggcggcggcagccagaagggcgatcagaatcctcagatcgccgcccacgtgatcagcgaggcctctagcaagaccacatccgtgctgcagtgggccgagaagggctactataccatgagcaacaatctggtgacactggagaatggcaagcagctgaccgtgaagcggcagggcctgtactatatctacgcccaggtgacattctgcagcaacagagaggcctcctctcaggccccttttatcgcctctctgagcctgaagtctccaggccggttcgagagaatcctgctgagggcagcaaacacccacagctccgccaagccttgtggccagcagtctatccacctgggcggcgtgttcgagctgcagccaggagccagcgtgtttgtgaatgtgacagaccctagccaggtgtcccacggcaccggcttcaccagcttcggcctgctgaagctgggcggcggcggcagcgggggcggcggctccggagggggaggctctcagaaaggcgatcagaatccacagattgctgcccacgtgatctctgaggcctctagcaagaccacaagcgtgctgcagtgggctgaaaaaggatactataccatgtctaataatctggtgacactggagaatggcaagcagctgactgtcaagagacagggcctgtattacatctacgctcaggtgacattttgcagcaatagagaggcctcctctcaggctcccttcatcgcctccctgtctctgaagtcccctggcaggtttgagcgcatcctgctgagagccgccaatactcacagctccgccaagccatgtggacagcagtccattcacctgggcggcgtgttcgagctgcaaccaggagccagcgtgttcgtgaatgtgacagacccctctcaggtgagccacggcaccggcttcaccagcttcggcctgctgaagctgggtggcggcggcagcggcgggggcggctccggaggcggaggctctcagaagggcgatcagaatcctcagattgcagcccacgtgatctccgaggcctctagcaagaccacatctgtgctgcagtgggctgagaaaggctactataccatgtctaacaatctggtgacactggagaacggcaagcaactgactgtcaaaagacagggcctgtattatatctacgctcaagtgacattctgcagcaatcgggaggcctcctctcaggcacccttcatcgccagcctgtccctgaagtcccctggccggttcgagagaatcctgttaagagccgccaatacacacagctccgccaaaccatgtggacagcagagcatacacctgggcggcgtgttcgagctgcaaccgggagcctccgtgtttgtcaatgtgacagacccatcccaggtgtctcacggcaccggcttcaccagcttcggcctgctgaagctg

SEQ ID NO: 156 - protein, 961 aa, Artificial sequence
  TIGIT-Fc Hole IgG4-sc3x4-1BBL: in TSP 501V1 AA SEQUENCE
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 157 - DNA, 2883 bp, Artificial sequence
  TIGIT-Fc Hole IgG4-sc3x4-1BBL: in TSP 501V1 NA SEQUENCE
atgaccggcacaatcgagacaacaggcaacatctctgccgagaagggaggcagcatcgccctgcagtgccacctgagcagcaccacagcccaggtgacccaggtgaactgggagcagcaggaccagctgctggccatctccaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcacggaggaggaggatccggaggaggaggatccgagtctaagtatggaccaccatgccctccatgtccagcacctgagtttgagggaggaccttccgtgttcctgtttccccctaagccaaaggacacactgatgatctccaggacaccagaggtgacctgcgtggtggtggacgtgtctcaggaggatcccgaggtgcagttcaactggtacgtggatggcgtggaggtgcacaatgccaagaccaagcctagggaggagcagtttaactccacataccgcgtggtgtctgtgctgaccgtgctgcaccaggattggctgaacggcaaggagtataagtgtaaggtgagcaataagggcctgccaagctccatcgagaagaccatctccaaggcaaagggacagccaagggagcctcaggtgtatacactgccacccagccagtgcgagatgaccaagaaccaggtgagcctgtcctgtgccgtgaagggcttctacccatctgacatcgccgtggagtgggagagcaatggccagcccgagaacaattataagaccacacctccagtgctggactccgatggctctttctttctggtgtccaggctgacagtggataagtctcgctggcaggagggcaacgtgttttcttgtagcgtgatgcacgaggccctgcacaatcactacacccagaagtccctgtctctgagcctgggcaagggcggcggcggctccggaggaggaggaagcgccgcctcccctaggctgcgcgagggaccagagctgagcccagacgatccagcaggactgctggacctgaggcagggaatgttcgcacagctggtggcccagaacgtgctgctgatcgacggccctctgtcttggtacagcgatccaggactggcaggcgtgagcctgacaggcggactgtcctataaggaggataccaaggagctggtggtggcaaaggcaggcgtgtactacgtgttcttccagctggagctgaggagagtggtggcaggagagggatccggatctgtgagcctggccctgcacctgcagccactgcggagcgccgcaggagcagccgccctggccctgaccgtggacctgccacctgcatctagcgaggcacggaattctgccttcggctttcagggcagactgctgcacctgagcgccggacagaggctgggagtgcacctgcacacagaggcaagggcaagacacgcatggcagctgacacagggagcaaccgtgctgggactgttccgcgtgacccctgagatcccagcaggactgccatccccccggtctgagggcggcggcgggtccggaggaggaggatctggcggcggcggcagcgccgcctcccccaggctgcgcgagggacctgagctgtccccagacgatcctgccggcctgctggacctgagacagggaatgtttgcccagctggtcgctcagaacgtgctgctgattgacggccccctgtcctggtatagcgatcctggactggcaggcgtgtctctgacaggcggactgagttacaaagaagacactaaagaactggtcgtcgccaaagccggcgtgtactacgtgttcttccaactggagctgaggagggtcgtcgccggcgaaggcagcggctctgtgagcctggccctgcacctgcaaccactgaggagcgccgccggcgccgccgccctggccctgactgtggacctgccaccagcatcctctgaggcaaggaattccgccttcggcttccagggccggctgctgcacctgtctgccggacagagactgggcgtccacctgcataccgaagccagagccaggcatgcctggcagctgacccagggcgccaccgtgctgggcctgttcagagtgaccccagaaattccagcaggactgccttccccaaggtctgagggaggaggaggaagtggcggaggaggatccggagggggagggagcgccgcctccccaagactgagagagggaccagagctgtcccctgatgaccctgccgggctgctggacctgagacaaggcatgttcgcccagctggtcgcacaaaacgtgctgttaattgacggcccactgtcctggtattccgaccctggcctggccggcgtgtccctgacaggcggcctgtcttacaaagaagacacaaaagaactggtcgtcgctaaagctggcgtgtactacgtgttcttccaattagaactgagaagggtcgtcgccggcgagggcagcgggtctgtgagcctggccctgcacctgcaaccgctgcggagcgccgccggcgctgccgccctggccctgacagtggacctgcctccagcaagctccgaggcaaggaatagcgccttcggctttcaaggccgcctgctgcacctgtccgccggacagcggctgggcgtccacctgcacaccgaagccagagcccgccatgcctggcagctgactcagggcgctaccgtgctgggcctgttccgcgtgaccccagagatccctgccggcctgccctcccctcggtctgag

SEQ ID NO: 158 - protein, 845 aa, Artificial sequence
  TIGIT-Fc Hole IgG4-sc3xCD40L: in TSP 503V1 AA SEQUENCE
MTGTIETTGNISAEKGGSIALQCHLSSTTAQVTQVNWEQQDQLLAISNADLGWHISPSFKDRVAPGPGLGLTLQSLTVNDTGEYFCIYHTYPDGTYTGRIFLEVLESSVAEHGGGGSGGGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGKGGGGSGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGGGGSGGGGSGGGGSQKGDQNPQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 159 - DNA, 2535 bp, Artificial sequence
  TIGIT-Fc Hole IgG4-sc3xCD40L: in TSP 503V1 AA SEQUENCE
atgaccggcacaatcgagacaacaggcaacatctctgccgagaagggaggcagcatcgccctgcagtgccacctgagcagcaccacagcccaggtgacccaggtgaactgggagcagcaggaccagctgctggccatctccaatgccgatctgggctggcacatcagcccctcctttaaggatagggtggcacctggaccaggcctgggcctgaccctgcagagcctgaccgtgaatgacacaggcgagtacttctgtatctaccacacatatcctgatggcacctatacaggcagaatctttctggaggtgctggagtctagcgtggccgagcacggaggaggaggcagcgggggaggaggctccgagtctaagtacggaccaccatgccctccatgtcctgcaccagagttcgagggaggaccatccgtgttcctgtttccacctaagcctaaggacaccctgatgatcagccggaccccagaggtgacatgcgtggtggtggacgtgagccaggaggaccccgaggtgcagtttaattggtatgtggatggcgtggaggtgcacaacgccaagaccaagcccagggaggagcagttcaacagcacctaccgcgtggtgtccgtgctgacagtgctgcaccaggactggctgaatggcaaggagtataagtgtaaggtgtccaacaagggcctgcctagctccatcgagaaaaccatcagcaaggcaaagggacagccacgggagccacaggtgtacaccctgccaccaagccagtgcgagatgacaaagaatcaggtgagcctgtcctgtgccgtgaagggcttttacccttccgacatcgccgtggagtgggagtctaacggccagccagagaacaattataagaccacacctccagtgctggactccgatggctctttctttctggtgtctaggctgaccgtggataagagccgctggcaggagggcaacgtgttcagctgcagcgtgatgcacgaggccctgcacaaccactatacacaaaagtccctgtctctgagcctgggcaagggcggcggcggcagcggcggaggaggctccggaggcggcggctctggcggcggcggcagccagaagggcgatcagaatcctcagatcgccgcccacgtgatcagcgaggcctctagcaagaccacatccgtgctgcagtgggccgagaagggctactataccatgagcaacaatctggtgacactggagaatggcaagcagctgaccgtgaagcggcagggcctgtactatatctacgcccaggtgacattctgcagcaacagagaggcctcctctcaggccccttttatcgcctctctgagcctgaagtctccaggccggttcgagagaatcctgctgagggcagcaaacacccacagctccgccaagccttgtggccagcagtctatccacctgggcggcgtgttcgagctgcagccaggagccagcgtgtttgtgaatgtgacagaccctagccaggtgtcccacggcaccggcttcaccagcttcggcctgctgaagctgggcggcggcggcagcgggggcggcggctccggagggggaggctctcagaaaggcgatcagaatccacagattgctgcccacgtgatctctgaggcctctagcaagaccacaagcgtgctgcagtgggctgaaaaaggatactataccatgtctaataatctggtgacactggagaatggcaagcagctgactgtcaagagacagggcctgtattacatctacgctcaggtgacattttgcagcaatagagaggcctcctctcaggctcccttcatcgcctccctgtctctgaagtcccctggcaggtttgagcgcatcctgctgagagccgccaatactcacagctccgccaagccatgtggacagcagtccattcacctgggcggcgtgttcgagctgcaaccaggagccagcgtgttcgtgaatgtgacagacccctctcaggtgagccacggcaccggcttcaccagcttcggcctgctgaagctgggtggcggcggcagcggcgggggcggctccggaggcggaggctctcagaagggcgatcagaatcctcagattgcagcccacgtgatctccgaggcctctagcaagaccacatctgtgctgcagtgggctgagaaaggctactataccatgtctaacaatctggtgacactggagaacggcaagcaactgactgtcaaaagacagggcctgtattatatctacgctcaagtgacattctgcagcaatcgggaggcctcctctcaggcacccttcatcgccagcctgtccctgaagtcccctggccggttcgagagaatcctgttaagagccgccaatacacacagctccgccaaaccatgtggacagcagagcatacacctgggcggcgtgttcgagctgcaaccgggagcctccgtgtttgtcaatgtgacagacccatcccaggtgtctcacggcaccggcttcaccagcttcggcctgctgaagctg

SEQ ID NO: 160 - protein, 244 aa, Artificial sequence
  TIGIT full length (Q495A1 Uniprot ID) AA SEQUENCE
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 161 - protein, 598 aa, Artificial sequence
  LILRB2 D1-4, full length (Q8N423-1 UniParc) AA SEQUENCE
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 162 - protein, 697 aa, Artificial sequence
  Siglec 10 full length (Q96LC7-1 Uniprot) AA SEQUENCE
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 163 - protein, 261 aa, Artificial sequence
  CD40L full length (P29965 Uniprot) AA SEQUENCE
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 164 - protein, 120 aa, Artificial sequence
  TIGIT Full ECD AA SEQUENCE
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 165 - protein, 440 aa, Artificial sequence
  LILRB2 Full ECD AA SEQUENCE
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGV

SEQ ID NO: 166 - DNA, 1422 bp, Artificial sequence
  sc3xCD40L, AA 114-261 C194S (P29965 Uniprot) internal linker 3x(GGGGS) NA SEQUENCE
cagaagggcgatcagaacccccagatcgccgcccacgtgatctccgaggcctctagcaagaccacatctgtgctgcagtgggccgagaagggctactatacaatgagcaacaatctggtgaccctggagaacggcaagcagctgacagtgaagcggcagggcctgtactatatctatgcccaggtgaccttctgctccaatagagaggcctcctctcaggccccctttatcgcctccctgtctctgaagtctcctggccggttcgagagaatcctgctgagggcagcaaacacacacagctccgccaagccctgtggccagcagtctatccacctgggcggcgtgttcgagctgcagccaggagccagcgtgtttgtgaatgtgaccgaccctagccaggtgtcccacggcaccggcttcaccagcttcggcctgctgaagctgggcggcggcggctctggcggaggaggcagcggaggaggaggctcccagaaaggcgatcagaaccctcagattgctgcccacgtgatcagcgaggcctctagcaagaccacatccgtgctgcagtgggctgaaaaaggatactatacaatgtctaataatctggtgaccctggagaatggcaagcagctgaccgtcaagagacagggcctgtattatatctacgcccaggtgaccttttgcagcaatcgcgaggcctcctctcaggctccattcatcgccagcctgtccctgaagtccccaggcaggtttgagcgcatcctgctgagagccgccaatacccacagctccgccaagccatgtggacagcagtccattcacctgggcggcgtgttcgagctgcaaccaggagccagcgtgttcgtgaatgtgaccgacccctctcaggtgagccacggcaccggcttcaccagcttcggcctgctgaagctgggtggaggaggctctggcgggggcggcagcggcggaggaggctcccagaagggcgatcagaatcctcagattgccgcccacgtgatctctgaggcctctagcaagaccacaagcgtgctgcagtgggcagaaaaagggtactatacaatgtctaacaatctggtgaccctggagaacggcaagcaactgaccgtcaaaagacagggcctgtactacatctacgctcaggtgaccttctgcagcaatagggaggcctcctctcaggcaccttttatcgcctctctgagcctgaagtccccaggccggttcgagagaatcctgttaagggcagcaaacactcacagctccgccaaaccatgtggacagcagagcatacacctgggcggcgtgttcgagctgcaaccgggagcctccgtgtttgtcaatgtgaccgacccatcccaggtgtctcacggcaccggcttcaccagcttcggcctgctgaagctg